SEQUENCE LISTING (2 sequences)

SEQ ID NO: 1 - protein, 300 aa, Zea mays
MPVILREMEDHSVHPMSKSNHGSLSGNGYEMKHSGHKVCDRDSSSESDRSHQEASAASESSPNEHTSTQSDNDEDHGKDNQDTMKPVLSLGKEGSAFLAPKLHYSPSFACIPYTSDAYYSAVGVLTGYPPHAIVHPQQNDTTNTPGMLPVEPAEEPIYVNAKQYHAILRRRQTRAKLEAQNKMVKNRKPYLHESRHRHAMKRARGSGGRFLNTKQLQEQNQQYQASSGSLCSKIIANSIISQSGPTCTPSSGTAGASTAGQDRSCLPSVGFRPTTNFSDQGRGGLKLAVIGMQQRVSTIR

SEQ ID NO: 2 - protein, 126 aa, Zea mays
MMIPARHMPPTMIVRNGGAAYGSSSALSLGQPNLMDNQQLQFQQALQQQHLLLDQIPATTAESCDNTGRGGGGRGSDPLADEFESKSGSENVDGVSVDDQDDPNQRPSKKKRYHRHTLHQIQEMEA

(19) United States
(12) Patent Application Publication
(10) Pub. No.: US 2023/0078124 A1
(43) Pub. Date: Mar. 16, 2023

(54) COMPOSITIONS AND METHODS FOR IMPROVING PLANT NITROGEN UTILIZATION EFFICIENCY (NUE) AND INCREASING PLANT BIOMASS

(71) Applicant: New York University, New York, NY (US)

(72) Inventors: Gloria Coruzzi, New York, NY (US); Chia-Yi Cheng, Taipei (TW)

(21) Appl. No.: 17/818,968

(22) Filed: Aug. 10, 2022

Related U.S. Application Data
(60) Provisional application No. 63/232,060, filed on Aug. 11, 2021.

(51) Int. Cl. C12N 15/82

(52) U.S. Cl. CPC C12N 15/8218; C12N 15/8261

(57) ABSTRACT

Provided are machine learning methods for identifying genes that affect plant properties. Also provided are plant cell sand plants comprising genetic modifications that improve plant nitrogen utilization and increased biomass. Methods of making the modified plant cells and plants are also provided.

### CROSS REFERENCE TO RELATED APPLICATIONS

This application claims priority to U.S. provisional application No. 63/232,060, filed Aug. 11, 2021, the entire disclosure of which is incorporated herein by reference.

### STATEMENT REGARDING FEDERALLY SPONSORED RESEARCH

This invention was made with government support under Grant Number IOS-568 1339362, awarded by the National Science Foundation, and Grant Number 1013620, awarded by the United States Department of Agriculture. The government has certain rights in the invention.

### SEQUENCE LISTING

The instant application contains a Sequence Listing which has been submitted in XML format and is hereby incorporated by reference in its entirety. Said XML copy, created on Aug. 8, 2022, is named “058636.00536.xml”, and is 3,084 bytes in size.

### BACKGROUND

Being able exploit genomic data to predict organismal outcomes in response to changes in nutrition, toxin and pathogen exposure could inform crop improvement, disease prognosis, epidemiology, and public health. To this end, machine learning methods have been developed and applied to infer phenotypes from genomic and epigenetic features associated with such conditions using changes in mRNA/protein expression levels, single nucleotide polymorphisms, chromatin modifications, and more. Despite the compelling motivation and cumulative efforts, accurately predicting complex phenotypic traits from genome-scale information remains both a promise and a challenge. Several factors contribute to these challenges. First, in contrast to the increasing availability of omics data, collection of high-quality phenotypic data from a genetically diverse population that adequately represents the phenotypic diversity space has become a major limiting factor¹. In addition, phenotypic data is often collected from experiments that are distinct from those used to acquire the functional genomics data. To overcome these limitations, phenotyping efforts should be expanded and performed on the same materials that are the source of genetic/genomic information². Furthermore, the explosion of omics data means that the features (e.g. numbers of genes) collected from a single experiment inevitably outnumber the phenotype space (e.g. sample size), leading to problems in data sparsity, multicollinearity, multiple testing, and overfitting³. This can be counteracted with increasing sample size, dimension reduction, or feature selection methods such as Principal Component Analysis (PCA), Least Absolute Shrinkage and Selection Operator (LASSO) regularization, Canonical Correlation Analysis (CCA), and so forth⁴. Additionally, cross-species approaches have been adopted in machine learning context to improve the performance of model-to-human knowledge translation⁵. Thus, there is an ongoing and unmet need to provide improved methods for analyzing genomic data to predict organismal outcomes in response to environmental changes, and use the results from the analysis to identify and modify genes to improve plant function. The present disclosure is pertinent to these needs.

### BRIEF SUMMARY

The present disclosure addresses a number of previous challenges in identifying and modifying genes to improve plant function by using an evolutionarily informed machine learning approach that exploits genetic diversity both within and across species. We employ transcriptome data of nitrogen response genes to predict nitrogen use efficiency (NUE), an agronomic outcome critical for worldwide food safety and sustainability^{2,6}. Nitrogen (N)—the main limiting macronutrient for plant growth—is supplemented in agricultural systems through application of N fertilizer. For major row crops such as maize (Zea mays), less than 40% of supplied N is taken up by the plants, while more than 60% of soil N is lost to the atmosphere or water bodies through multiple processes such as denitrification, ammonia volatilization, leaching etc⁷. Balancing the need to further increase crop yields, while also mitigating the environmental impacts associated with N fertilizer, is a challenge for sustainable agriculture. Considering the polygenic nature of NUE that involves the integration of developmental, physiological, and metabolic processes², machine learning was applied as a strategy to tackle the mechanisms underlying this complex trait. To this end, we collected transcriptomic and phenotypic NUE data from two species—maize (a crop) and Arabidopsis (a model)—each of which included a panel of genotypes with diverse genetic background and NUE variation. We used genes whose response to N-treatments (N-DEGs) was conserved within and across species as a dimension reduction approach for machine learning. As maize and Arabidopsis are highly divergent phylogenetically, these evolutionarily conserved N-response genes should represent essential/core functions contributing to NUE. We show that models constructed using these evolutionarily conserved N-DEGs significantly improved the prediction of NUE traits from gene expression values, compared to an equal number of top ranked N-DEGs or randomly selected expressed genes. The inclusion of the model species Arabidopsis enabled us to validate using mutants. This evidence validated that the genes whose expression levels are important in predicting NUE in the machine learning models are more than just markers, but functionally required for the trait. Moreover, we show that the described evolutionarily informed machine learning pipeline is transferable to other species and traits in plants and animals. Specifically, application of the described method to other matched transcriptome and phenotype datasets related to drought in field grown rice or disease in mouse models resulted in enhanced prediction accuracies of the learned models. As such, the described evolutionarily informed machine learning pipeline has the potential to identify genes of importance for complex phenotypes of interest across biology, agriculture, or medicine.

A result of the described analysis identified maize genes that can be modulated to improve plant function. In particular, the present disclosure shows that expression of certain identified genes can positively affect nitrogen utilization and increase plant biomass, including but not necessarily limited to maize grain mass. As such, the disclosure provides for inhibiting the expression and/or function of one or a combination of transcription factors (TFs) described herein. In embodiments, the expression and/or function of hb75, alone or in combination with another described TF, such as nf-ya3, is provided for use in improving plant function.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. Evolutionarily informed machine learning approach enhances the predictive power of gene-to-phenotype relationships. Step 1 Feature selection: Phenotypic and transcriptomic data of N-responses were generated from Arabidopsis (lab-grown) and maize (field-grown) under low- vs. high-N conditions. The expression levels of N-response differentially expressed genes (N-DEGs) conserved in both species were identified via ‘leave-out-one’ approach (FIG. 4) and used as gene features in the machine learning methods in Step 2. This biologically principled approach to reduce the feature dimensions ultimately improved the model performance (Table 1). Step 2 Feature importance: We ranked the genes based on i) the XGBoost-derived feature importance score (left) and ii) the TF connectivity in a GENIE3 regulatory network (right) constructed from the N-response TFs (Step 1) as regulators and the XGBoost important features as targets. Step 3 Feature validation: We validated the role of NUE for eight TFs in planta using Arabidopsis and maize loss-of-function mutants.

FIGS. 2a-2c. Nitrogen is the leading factor explaining the NUE variation across Arabidopsis natural accessions. (2a) Boxplot of NUE among the Arabidopsis genotypes measured in three independent batches. The coefficients of variation demonstrate the broad range of phenotype of this panel of genotypes, which has been widely used in NUE studies. The X-axis is ordered in the increasing value of average NUE. In the box plots, the box represents the 25th to 75th percentile and the line within the box marks the median. Whiskers above and below the box indicate the 10th and 90th percentiles. Points above and below the whiskers indicate outliers outside 10th and 90th percentiles. (2b) The correlation of traits measured in this study. NUE at the pre-bolting stage is highly correlated with NUpE. Biomass, g/plant; N uptake, mg N/plant; N %, N uptake/Biomass; E %, 15N uptake/N uptake; NUE, Biomass/applied N; NUpE, 15N uptake/applied 15N; NUtE, Biomass/N uptake. (2c) The NUE variation is primarily explained by nitrogen levels, followed by accession and nitrogen by accession interaction. Two-way ANOVA P-value: G, <2E-16; N, <2E-16; Gx N, 9.93E-07. For each genotype n>10 biologically independent plants examined over three independent experiments.

FIGS. 3a-3c. Genotype is the leading factor explaining the NUE variation in maize breeding lines. (3a) Boxplot of Total nitrogen utilization (NUtE) values among the maize genotype panel measured in three consecutive years. The X-axis is ordered by increasing value of average Total NUtE. The coefficients of variation demonstrate the broad range of phenotype of this smaller panel of maize genotypes, which spans the distribution of NutE values measured in a larger representative germplasm collection (FIG. 8). In the box plots, the box represents the 25th to 75th percentile and the line within the box marks the median. Whiskers above and below the box indicate the 10th and 90th percentiles. Points above and below the whiskers indicate outliers outside 10th and 90th percentiles. (3b) The correlation of traits measured in this study. (3c) The total NUtE variance of 2014, the year when the RNA samples were harvested, is primarily explained by Genotype (G), followed by N, and Gx N effect. Two-way ANOVA P-value: G, 8.6E-11; N, 2.9E-13; G×N, 2.28E-07. For each genotype n>5 biologically independent plants examined over three independent experiments.

FIG. 4. Evolutionarily conserved N-response genes across Arabidopsis-maize used as a biologically principled feature reduction method for the XGboost machine learning pipeline. The RNA-seq reads from leaves of Arabidopsis and maize N-treated samples were aligned to reference genome assemblies using BBMap and the read counts were generated using featureCounts. The N-response DEGs (N-DEGs) were identified using generalized linear models in edgeR and leave-out-one method: one genotype (out of 18) was left out during each round of analysis and the intersection of 18 DEG lists was used for feature reduction (For details, see FIG. 10). The overlap of N-DEGs from Arabidopsis (n=2,123) with maize (n=6,914) resulted in a set of evolutionarily conserved N-response Arabidopsis genes (n=610) which were used as features in the machine learning model. The corresponding conserved N-response genes in maize were further intersected with genes responding to nitrogen by genotype effects (n=3,664), resulting in 248 maize genes that were used as features in the machine learning model to predict NUE.

FIG. 5. Evolutionarily informed machine learning models uncover genes-of-importance and predictive of NUE. Step 1. The evolutionarily conserved N-DEGs between Arabidopsis and maize (see FIG. 4) and NUE data from n genotypes are split into training (n-1 genotypes) and test (left-out genotype) set (for details see FIG. 10). Step 2. The training set was used to optimize the XGBoost model, which then predicts the NUE using the gene expression in the test set. Step 3. The model performance was evaluated by calculating the Pearson's correlation coefficient r between the predicted and actual NUE values. In Arabidopsis, the dots indicate the Pearson's r of 100 individual iterations and the pointranges indicate mean+/−SD. In maize, there are only two data points for each genotype thus the Pearson's r was calculated from the pooled predicted and actual NUE from 100 iteration. Step 4. The TF features were ranked based on their contribution to the NUE. Certain of the genes are functionally validated in this disclosure.

FIGS. 6a-6c. Experimental validation of candidate TFs in NUE using loss-of-function mutants for Arabidopsis (lab) and maize (field). (6a) The Arabidopsis T-DNA mutants (Methods) in group I genes displayed higher NUE compared to wild-type under N-replete (yellow, 10 mM KNO₃) and N-deplete (grey, 2 mM KNO₃) conditions. This suggests their non-redundant role(s) in regulating NUE regardless of the environmental N levels. (6b) The Arabidopsis mutants in group II genes displayed higher NUE specifically under N-deplete conditions. This indicates that the group II genes are either only required under N-deplete conditions or are functionally redundant under N-replete conditions. The experiments were carried out three times with 10 or more plants per genotype per condition. (6c) Changes in NUE and component traits for the maize nfya3-1::Mu mutant compared to wild-type W22. Plants were grown in the field supplied additional N (150 kg N fertilizer/ha). Trait values are the average of five plants sampled from each of three replicate field plots, 15 plants per genotype (Methods). The higher total NUtE observed in the mutant was a combinatorial effect of lower stalk N (g/plant) (P=0.002), total N uptake (P=0.05) and higher grain biomass (P=0.1). The increased NUE phenotype was also observed in the Arabidopsis T-DNA mutant defective the homolog gene NF-YA6 (AT3G14020) (b). The pointrange indicates mean+/−SD. The P-value was calculated between WT and indicated mutant allele using one-sided t-test with unequal variance.

FIG. 7. Distribution of nitrogen utilization values among U.S. Corn Belt inbred diversity and the genotypes chosen for transcriptome-based prediction of this trait.

FIGS. 8a-8d. Schematic overviews of plant growth conditions and N-treatments.

FIG. 9. In maize, total NUtE is an optimal measure of NUE, compared to grain NUtE, the latter of which is confounded by maturity.

FIGS. 10a-10c. Comparison of XGBoost models created using a unified list of gene features (10a), or independent lists of gene features (10b). FIG. 10C provides a comparison of Arabidopsis and Mainze genotupes and correlation coeefieicents.

FIGS. 11a-11b. XGBoost-based feature importance ranking is marginally correlated with the edgeR-based P-value ranking.

FIGS. 12a-12b. The conserved N-DEGs can be used to predict multiple traits.

FIGS. 13a-13c. The Arabidopsis gene feature importance ranking is trait specific.

FIG. 14a-14c. The Arabidopsis gene feature importance ranking is trait specific.

FIG. 15. Use case: the pipeline proposed in this study can be applied on a different data set.

FIG. 16. Validation of candidate TFs in NUE using loss-of-function mutants in Arabidopsis.

FIG. 17. Expression of target genes in plant loss-of-function mutants used in this study.

### DETAILED DESCRIPTION

Every numerical range given throughout this specification includes its upper and lower values, as well as every narrower numerical range that falls within it, as if such narrower numerical ranges were all expressly written herein.

As used in the specification and the appended claims, the singular forms “a” “and” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about” it will be understood that the particular value forms another embodiment. The term “about” in relation to a numerical value is optional and means for example+/−10%.

This disclosure includes every amino acid sequence described herein and all nucleotide sequences encoding the amino acid sequence. Polynucleotide and amino acid sequences having from 80-99% similarity, inclusive, and including and all numbers and ranges of numbers there between, with the sequences provided here are included in the invention. All of the amino acid sequences described herein can include amino acid substitutions, such as conservative substitutions, that do not adversely affect the function of the protein that comprises the amino acid sequences. The disclosure includes all polynucleotide and amino acid sequences described herein, and every polynucleotide sequence referred to herein includes its complementary DNA sequence, and also includes the RNA equivalents thereof to the extent an RNA sequence is not given. Any sequence referred to by a database entry is incorporated herein by reference as the sequence exists in the database as of the effective filing date of this application or patent, including but not limited to database entries that are signified by an alphanumeric indicator that starts with “Zm.”

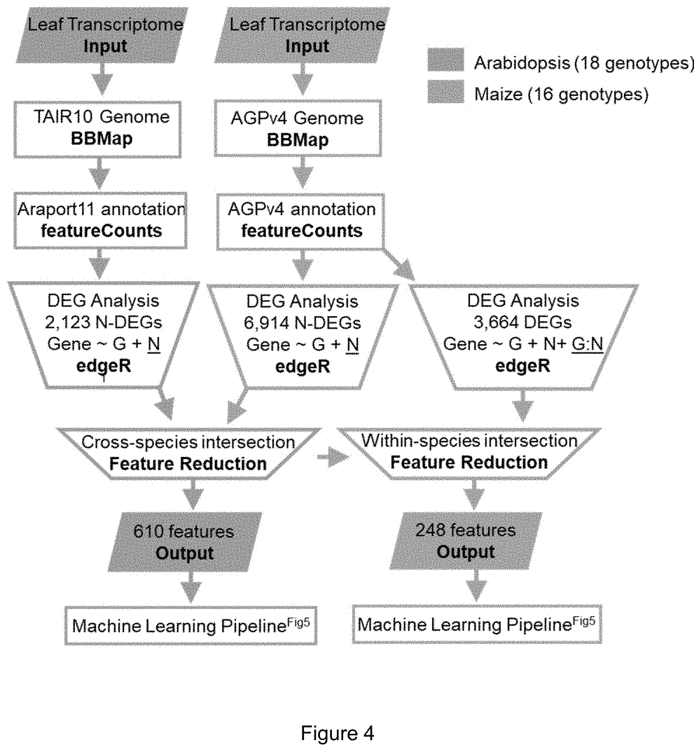

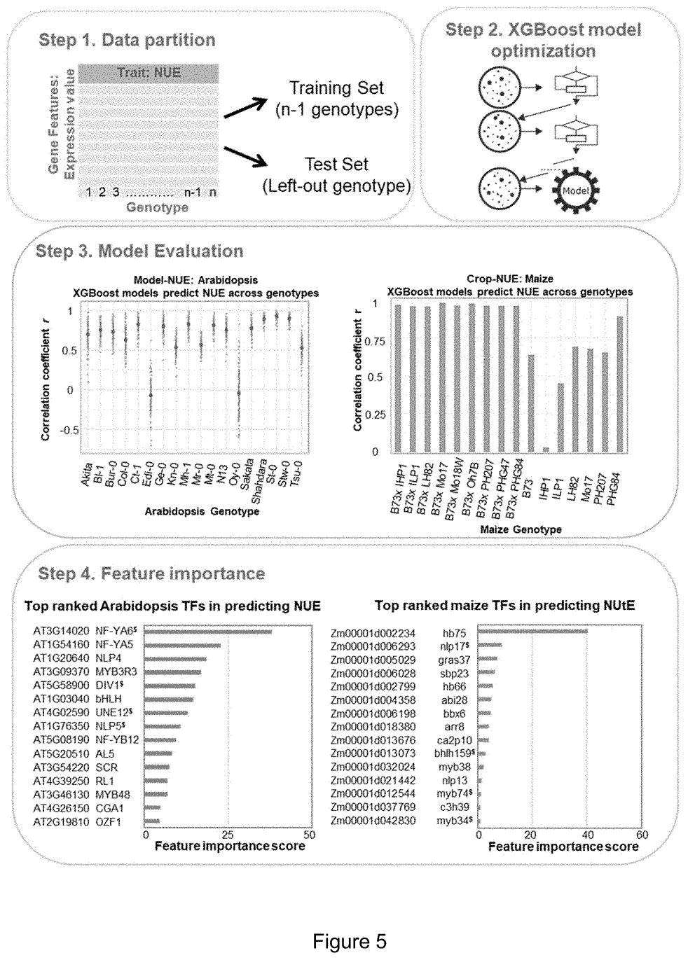

The disclosure includes all described methods of analyzing transcriptome data to predict a phenotype described herein, all machine learning approaches described herein that are used for analysis of gene expression changes using Nitrogen (N)-treatment that influences expression of N responsive genes (N-DEGs), and extensions of those approaches to different genes, their protein products, and interspecies comparisons of transcriptome analysis and predictions of the influence of transcription factors on any phenotype. In a non-limiting embodiment, the disclosure includes the process as depicted in FIG. 4 and its accompanying description, and extensions thereof to other types of plants, as well as non-plant organisms.

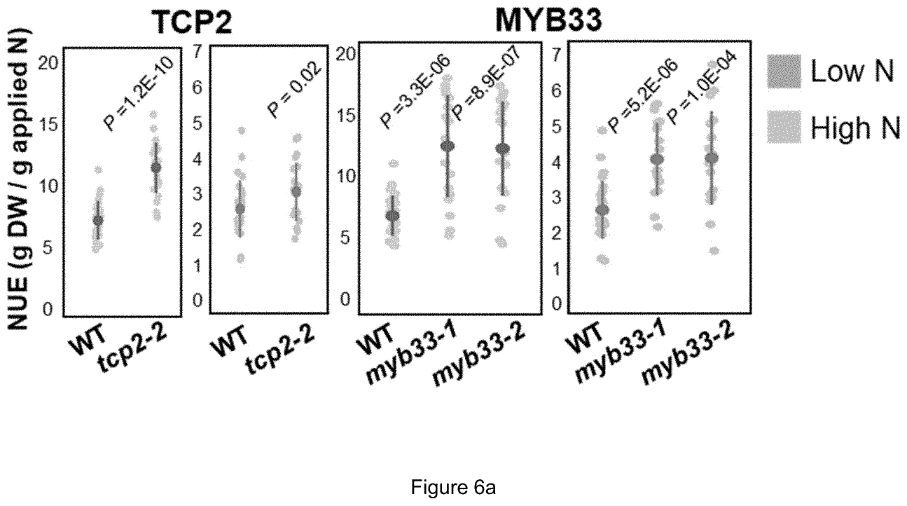

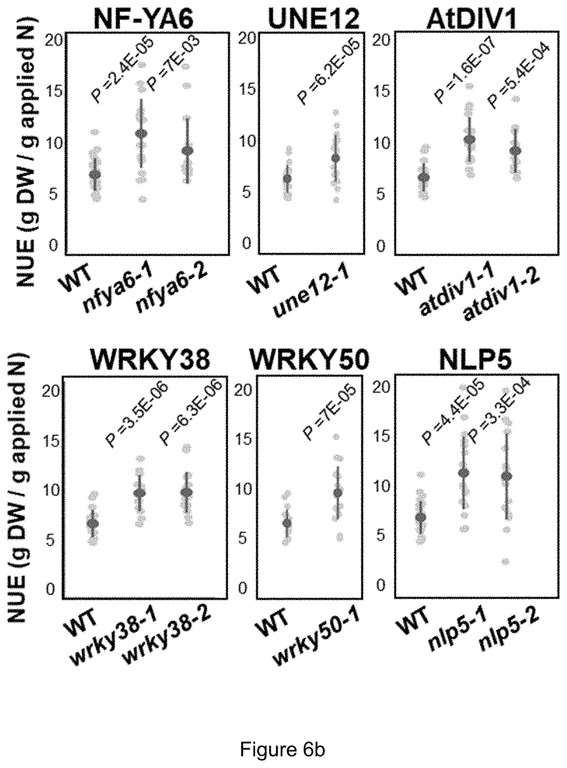

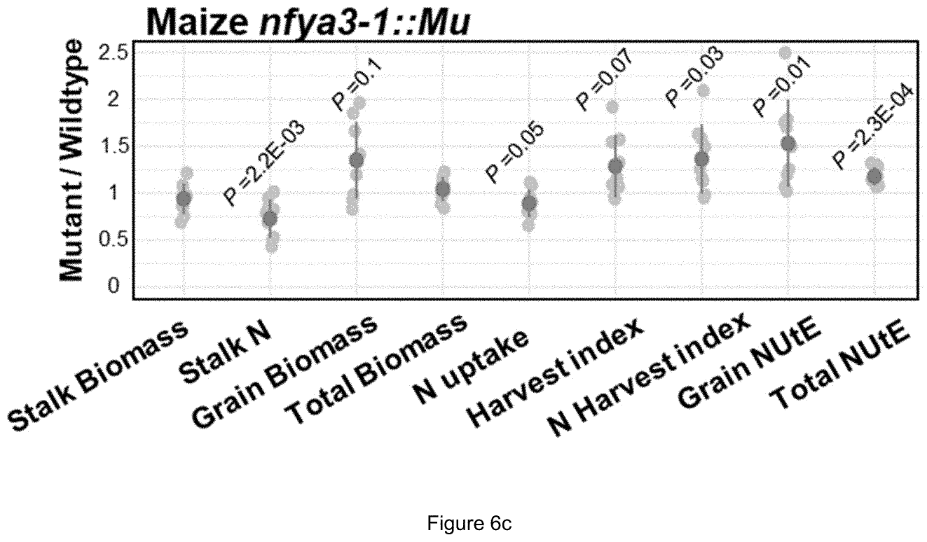

In embodiments, based at least in part on the described analysis, the present disclosure provides compositions and methods for modifying plants and/or plant cells. The compositions and methods relate to altering expression of one or a combination of the TFs. Altering the expression can result in any change in the plant described herein. In embodiments, practicing a method of the disclosure results in an increase in N uptake, increased biomass, such as increased grain biomass, an increased harvest index, an increased Total nitrogen utilization (NUtE), an increased total Grain NUtE, or a combination thereof. Non-limiting demonstrations of these effects are summarized in FIG. 6, panel c, and its accompanying text. For instance, mutating Maize nyfa3-1 results in the described effects shown in FIG. 6. In this regard, Table 4 provides an analysis of select TFs, and includes analysis of nf-ya3 (also referred to herein as nyfa3-1) Ranksum scores. The ranksum, as described further below, is the sum of three rankings for each TF based on i) the number of TF-gene targets involved in the N-assimilation pathways, ii) the number of TF-gene targets comprising gene features predictive of N utilization (NUE), and iii) the number of TF-gene targets that are also transcription factors. Without intending to be bound by any particular theory, it is considered the ranksum value provides an indication of the importance of the described TFs in terms of N-assimilation pathways and NUE. As can be seen from Table 4, the ranksum of nf-ya3 (46) is similar to hb75 (41). Thus, based on the data presented in FIG. 6, the ranksum value for nf-ya3, and the positive changes in plant properties that are related to mutation of nf-ya3, it is expected that mutation of hb75 will have similar effects on plant N uptake, increased grain biomass, increased harvest index, increased NUt, and total Grain NUtE as observed for mutating nf-ya3. Thus, the disclosure, in one embodiment, provides for disrupting or inhibiting the expression of hb75, nf-ya3, or a combination thereof, in plant cells. In embodiments, the disclosure provides modified plant cells and plants, wherein the only genomic modification comprises modification of one or two of the described genes. In embodiments, modification of only one, or only two, of the describes genes is sufficient to produce the described improved properties, relative to the same properties in plants that do not comprise the same modifications.

Notwithstanding the foregoing description, the TFs of the present disclosure include any TF that is referenced in the description (including tables) or in the figures. Overexpression and underexpression of any one or combination of the described genes is included in the disclosure. Overexpression of a particular gene can be accomplished by any method known in the art. For example, a plant cell may be transformed with a nucleic acid vector comprising the coding sequences of the desired gene operably linked to a promoter active in a plant cell such that the desired gene is expressed at levels higher than normal (i.e., levels found in a control/nontransgenic plant). The promoters can be constitutively active in all or some plant tissues or can be inducible. The under-expression of a desired gene can be accomplished by any method known in the art. For example, a gene may be knocked out, or mutated such that lower than normal levels of the gene product is produced in the transgenic cells or plant. For example, such mutations include frame-shift mutations or mutations resulting in a stop codon in the wild-type coding sequence, thus preventing expression of the gene product. Another exemplary mutation is the removal of the transcribed sequences from the plant genome, for example, by homologous recombination. Another method for under-expressing a gene is transgenically introducing an insertion or deletion into the transcribed sequence or an insertion or deletion upstream or downstream of the transcribed sequence such that expression of the gene product is decreased as compared to wild-type or appropriate control. Additionally, microRNA (native or artificial) can be used to target a particular encoding mRNA for degradation, thus reducing the level of the expressed gene product in the transgenic plant cell. Another method for underexpression of a gene of interest is using clustered regularly interspaced short palindromic repeats (CRISPR) gene inactivation. A variety of suitable CRISPR systems for use in plants can be used, and include but are not necessarily limited to Cas3, Cas9, and Cas13 based systems, all of which are known in the art and can be adapted for the described purposes, such as by using a suitable CRISPR enzyme and guide RNA to target the described gene(s) and/or their regulatory elements, such as promoters.

The sequence of the protein encoded by maize nf-ya3 is:

(SEQ ID NO: 1)
MPVILREMEDHSVHPMSKSNHGSLSGNGYEMKHSGH
KVCDRDSSSESDRSHQEASAASESSPNEHTSTQSDN
DEDHGKDNQDTMKPVLSLGKEGSAFLAPKLHYSPSF
ACIPYTSDAYYSAVGVLTGYPPHAIVHPQQNDTTNT
PGMLPVEPAEEPIYVNAKQYHAILRRRQTRAKLEAQ
NKMVKNRKPYLHESRHRHAMKRARGSGGRFLNTKQL
QEQNQQYQASSGSLCSKIIANSIISQSGPTCTPSSG
TAGASTAGQDRSCLPSVGFRPTTNFSDQGRGGLKLA
VIGMQQRVSTIR

The sequence of the protein encoded by maize hb75 is:

(SEQ ID NO: 2)
MMIPARHMPPTMIVRNGGAAYGSSSALSLGQPNLMD
NQQLQFQQALQQQHLLLDQIPATTAESCDNTGRGGG
GRGSDPLADEFESKSGSENVDGVSVDDQDDPNQRPS
KKKRYHRHTLHQIQEMEA.

Those skilled in the art will recognize how to identify and modify DNA sequences that encode the described proteins based on the genetic code.

The described compositions and methods can be used for any type of plant, such as monocots, dicots, gymnosperms, or plant cells. The term “plant cell” as used herein refers to protoplasts, gamete producing cells, and includes cells which regenerate into whole plants. Plant cells include but are not necessarily limited to cells obtained from or found in: seeds, suspension cultures, embryos, meristematic regions, callus tissue, leaves, roots, shoots, gametophytes, sporophytes, pollen, and microspores. Plant cells can also be understood to include modified cells, such as protoplasts, obtained from the aforementioned tissues. In non-limiting embodiments, the method is used for any species of woody, ornamental, decorative, crop, cereal, fruit, or vegetable plant. The method can be used on intact plants, isolated plant parts, and plant cells. In embodiments, the method is used with a seed, a suspension culture, an embryo, a meristematic plant region, callus tissue, a leaf, a root, a shoot, a gametophyte, a sporophyte, pollen, a microspore, or a protoplast. In embodiments, the plant or plant cells that are modified according to the disclosure are any member of the following genera/group: Artemisia, Acorns, Aegilops, Allium, Amborella, Antirrhinum, Apium, Arabidopsis, Arachis, Beta, Betula, Brassica, Cannabis, Capsicum, Ceratopteris, Citrus, Coffea, Cryptomeria, Cycas, Descurainia, Eschscholzia, Eucalyptus, Glycine, Gossypium, Hedyotis, Helianthus, Hordeum, Ipomoea, Lactuca, Linum, Liriodendron, Lotus, Oryza, Lupinus, Lycopersicon, Medicago, Mesembryanthemum, Nicotiana, Nuphar, Pennisetum, Persea, Phaseolus, Physcomitrella, Picea, Pinus, Poncirus, Populus, Prunus, Robinia, Rosa, Saccharum, Schedonorus, Secale, Sesamum, Solanum, Sorghum, Stevia, Thellungiella, Theobroma, Triphysaria, Triticum, Vitis, Zea, or Zinnia. In non-limiting embodiments, the modified plant or plant cells are from one or more so-called “elite” varieties of maize. The disclosure includes seeds produced by any modified plant herein, and progeny of the plants and seeds. Articles of manufacture comprising the seeds and a container that contains the seeds are also provided. In embodiments, the articles of manufacture comprise kits.

The following Examples are intended but not limit the disclosure.

### Example 1

We analyzed whether the prediction power of machine learning models could be enhanced by exploiting the genetic diversity of gene responses and phenotypes both within and across species. In non-limiting embodiments, we tested whether using N-DEGs conserved both within and across species as a biologically-principled means of dimension reduction, could enhance identification of genes of importance to predicting NUE phenotypes from gene expression data across a model (Arabidopsis) and crop (maize) plant. This model-to-crop machine learning approach enables more rapidly validation of conserved features of importance to NUE in the crop using the model species.

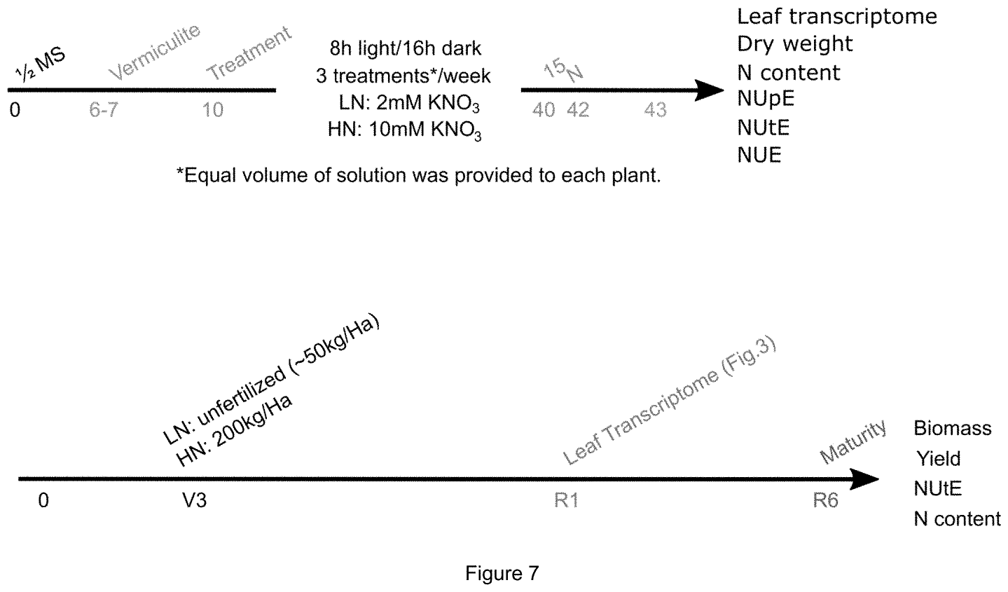

Within each species, we selected a set of genotypes that exhibit a broad spectrum of phenotypic variation in NUE. The data included 18 Arabidopsis accessions that were previously identified for their NUE diversity⁸ which originated from a nested collection of 265 accessions found in a wide range of habitats differing notably in soil nutrient richness⁹. The 23 maize genotypes analyzed in this disclosure correspond to 12 maize inbred lines and their 11 corresponding hybrids with B73. We selected these 12 maize inbred lines to represent the phenotypic diversity for NUE traits that we measured among a population of 318 field-grown maize inbreds (FIG. 7), which broadly represent the current germplasm base for U.S. Corn Belt hybrids. This maize population that we tested for NUE traits includes the parents of the Nested Association Mapping (NAM) population¹, improved inbreds from different breeding programs described in recently expired plant variety patents¹⁰, and the Illinois Protein Strains that display the known phenotypic extremes for NUE traits in maize²³. The B73 inbred maize line was chosen as the parent for the hybrids, because it is a major founder of the Stiff-Stalk heterotic group used in the production of nearly all commercial U.S. Corn Belt hybrids¹¹. Furthermore, B73 displays high nitrogen utilization efficiency (NUE), and also serves as the reference genome sequence assembly for maize¹².

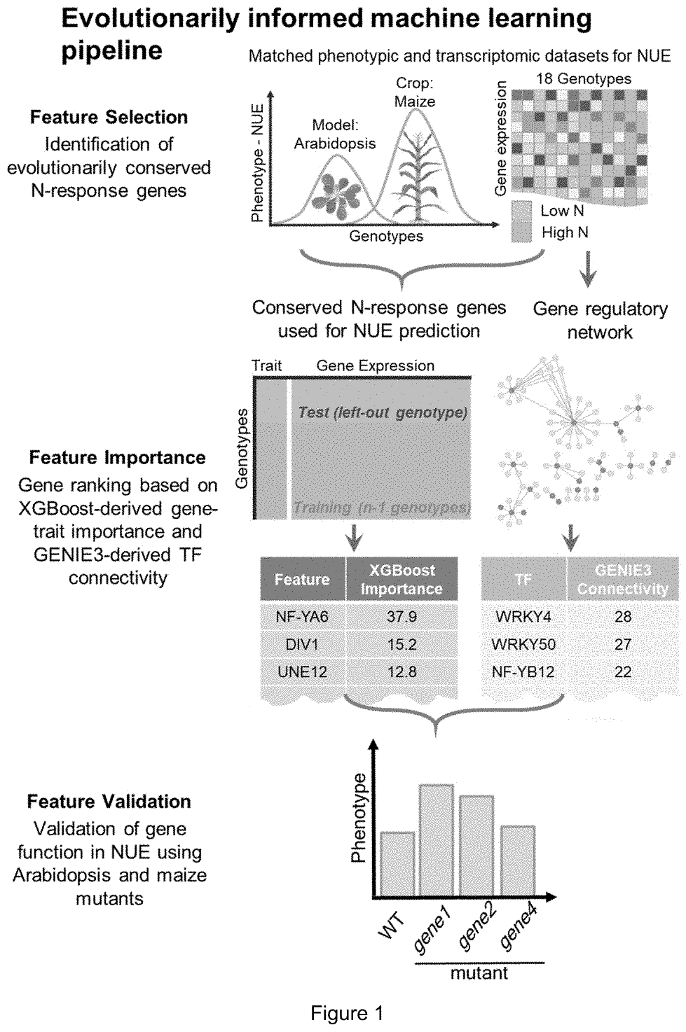

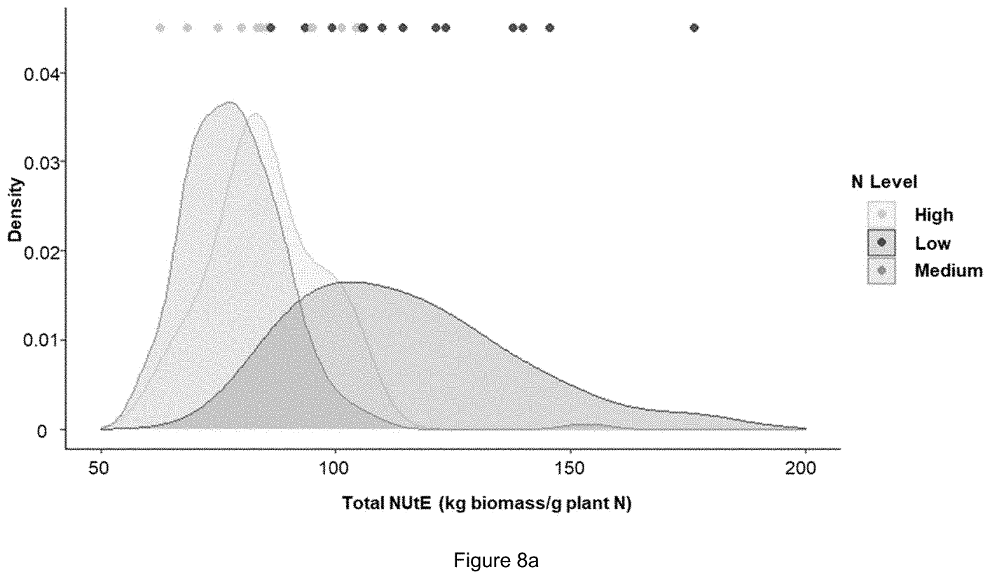

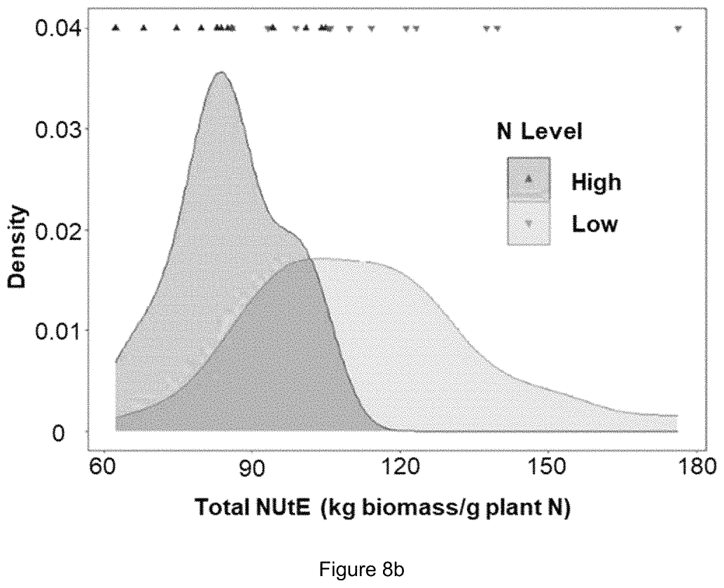

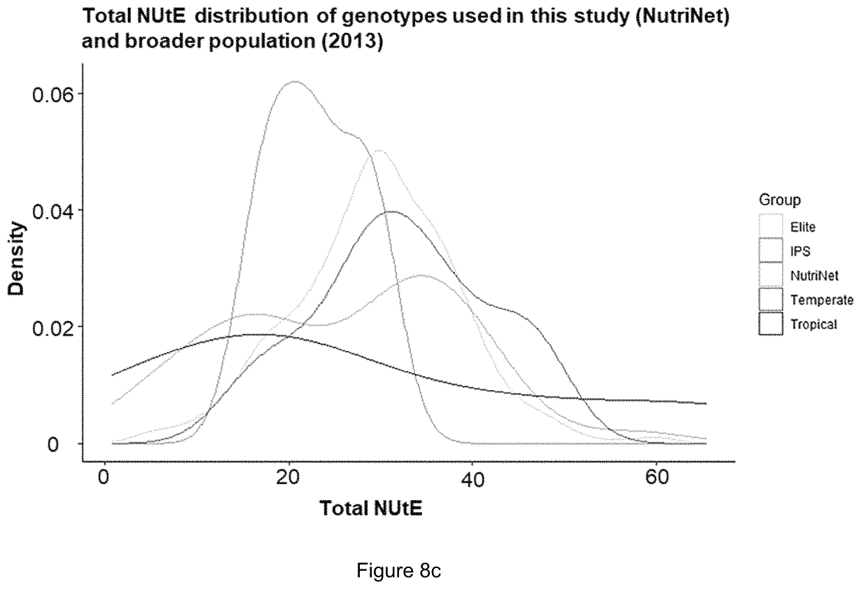

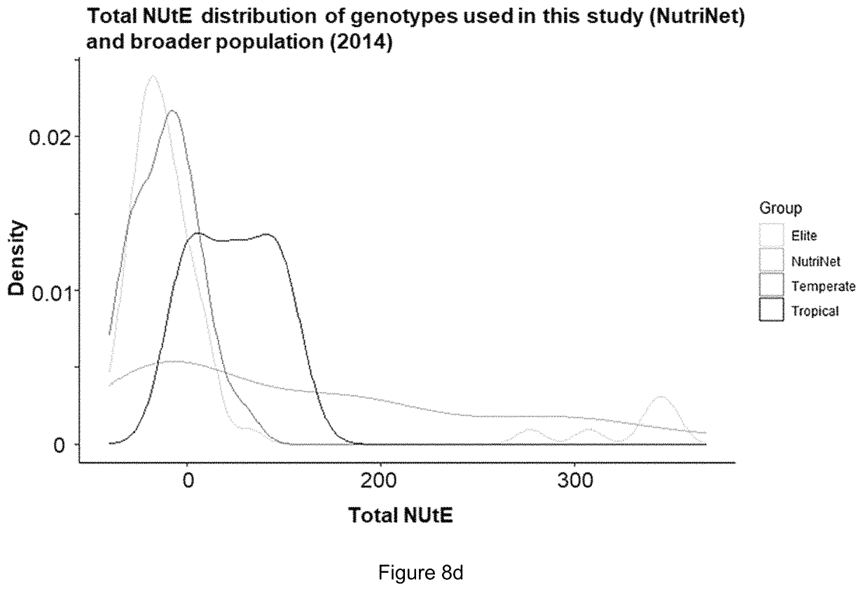

To test whether genome-wide responses to N-treatments evolutionarily conserved across the model and crop could be a biologically principled approach to enhance the model performance of predicting NUE, we constructed a three-step machine learning pipeline (FIG. 1). (Step I) Feature selection: First, we collected and analyzed matched phenotypic and transcriptomic data from the same replicate plants for each N-treatment conducted in a controlled laboratory setting (Arabidopsis) or field conditions (maize) and (FIG. 8). Using linear models, we identified N-response differentially expressed genes (N-DEGs) in parallel for maize and Arabidopsis, and retained the N-DEGs conserved both within and across species as gene features used in machine learning. (Step II) Feature importance: We selectively used the expression levels of these evolutionarily conserved N-DEGs as a biologically-principled approach to feature reduction in the gradient boosting-based method XGBoost¹³ predictive models. The outcome of the machine learning enabled ranking the N-DEGs whose expression levels best predicted the NUE traits measured in the same set of plants. Moreover, we identified the transcription factors (TF) regulating these genes of importance to NUE and measured their connectivity in the NUE network by constructing a NUE gene regulatory network (GRN) using a Random Forest-based method GENIE3¹⁴. Through integration of the results of these complementary means, we generated ranked lists of: i) gene features based on their contribution to the trait prediction (XGBoost-based importance score), and ii) TFs based on their level of connectivity in the GRN for each species (GENIE3-based connectivity). (Step III) Feature validation: we validated the function of eight candidate TFs in Arabidopsis or maize based on their importance score to the NUE trait and/or their degree of connectivity in the GRN. We experimentally confirmed the function of these eight TFs in regulation of NUE in planta using loss-of-function mutants in Arabidopsis, as well as in maize, where available.

### Example 2

### Quantifying NUE Phenotypes Across Arabidopsis and Maize Varieties

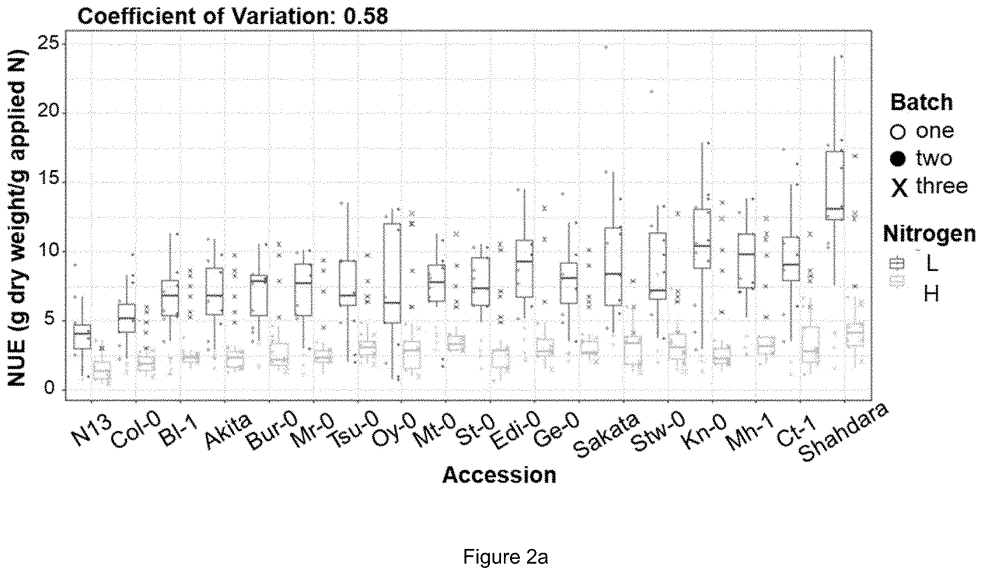

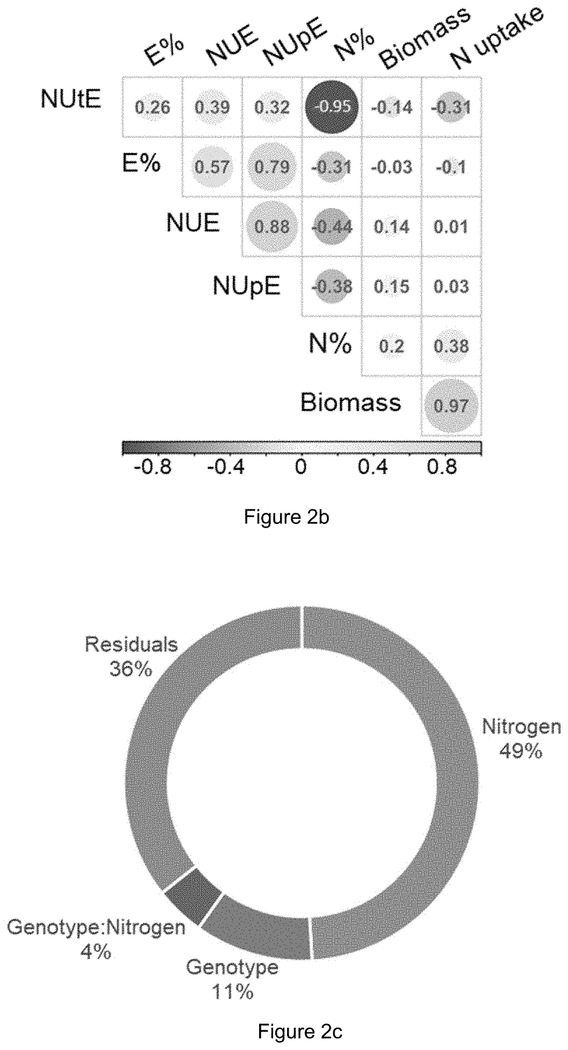

In the described phenotypic analysis, we quantified nitrogen use efficiency (NUE) as the efficiency of converting supplied N to biomass/grain yield. For Arabidopsis, NUE was calculated as the efficiency with which each plant converted supplied N into shoot biomass (NUE=Above ground dry weight/Applied N). This measure of NUE is achieved by providing each plant with a trackable/contained amount of N in pots in a lab setting, as a proxy for the field agricultural setting². Indeed, we found the Arabidopsis accessions previously selected for NUE diversity⁸ present a broad range of NUE variation in our own experiments, as evidenced by the coefficient of variation (CV=0.58) (FIG. 2a). The correlation of traits shows that NUE at the pre-bolting stage is highly correlated with NUpE (r=0.88), and to a lesser extent with NUtE (r=0.39) (FIG. 2b). The NUE variation among the Arabidopsis accessions is primarily explained by nitrogen levels, followed by accession and nitrogen-by-accession interaction (Two-way ANOVA P-value: G, <2E-16; N, <2E-16; G×N, 9.93E-07). This indicates the N-level explains the phenotypic variation in NUE in this collection of Arabidopsis ecotypes.

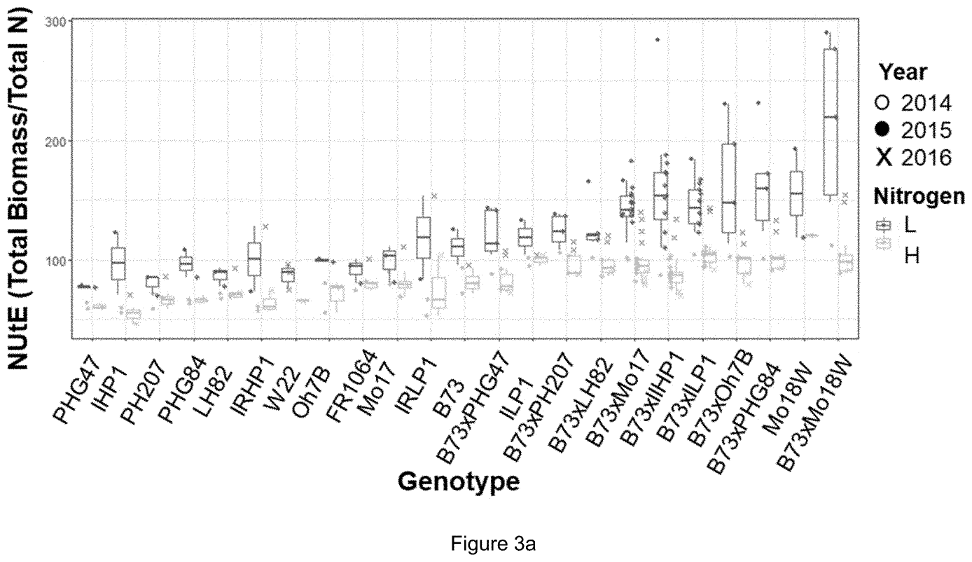

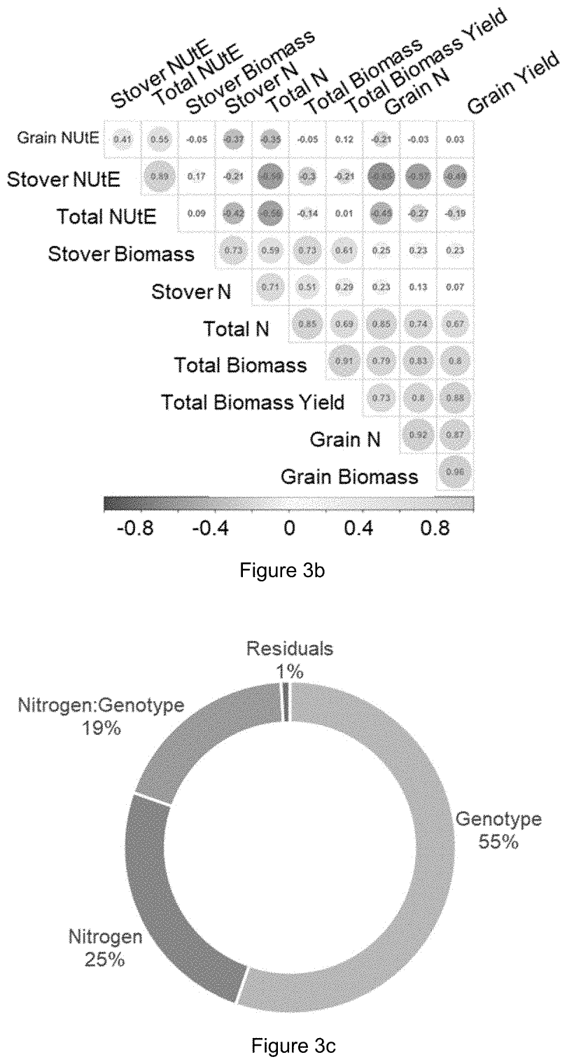

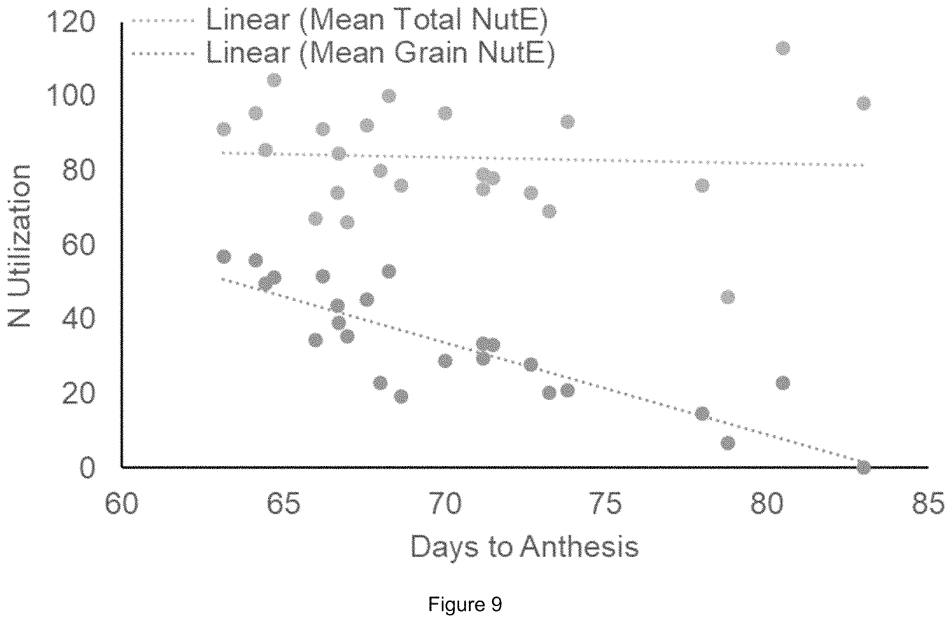

For field-grown maize, we used Total NUtE, (stover biomass+grain biomass)/(stover N content +grain N content), as the target trait (FIG. 3a). We chose this because Total NUtE is more robust to the effects of maturity and photoperiod in the field¹⁵ (FIG. 9), and remains highly correlated to grain NUtE (FIG. 3b). We measured total NUtE across 318 maize inbred lines in a field experiment where soil N supply was not limiting, and observed a nearly three-fold range in total NUtE (56-156 kg biomass/g plant N) (FIG. 7). To illustrate the influence of soil N-supply on total NUtE, 25 inbred maize lines chosen to represent both historical (NAM parents)¹ and elite genetic diversity¹⁰ were grown in adjacent plots that received either no N fertilizer or were N-fertilized as the larger population. When grown with sufficient N, the distribution of NUtE values for these 25 maize inbreds overlaps with that observed from the larger population of 318 maize genotypes (FIG. 8). In this disclosure, we selected 12 (from the 25 above) maize inbreds, which exhibited a similar coefficient of variation for NUtE phenotypic values (CV=0.19) as the larger population of 318 genotypes (CV=0.15) for matched transcriptome profiling and detailed phenotyping in N-responsive field plots, over three field seasons.

ANOVA results revealed that 55% of the total NUtE variation in this maize experiment was attributed to genetic effects (FIG. 3c). Our two-way ANOVA analysis of the maize data shows that in addition to G (P-value=8.6E-11) and N (P-value=2.9E-13), G×N was also a significant factor (P-value=2.28E-07) explaining 19% of the variation in Total NUtE (FIG. 3c). This is distinct from our findings for Arabidopsis, where N is the main explanatory variable (FIG. 2c). This difference likely reflects not only the overall greater genetic diversity in the maize varieties, but also suggests that intensive breeding and selection for N-responsive grain yields in maize¹⁶ may have expanded the phenotypic variation for NUE beyond that observed among the Arabidopsis natural accessions. We therefore included these interactions of maize genotype with nitrogen supply on the NUE phenotype as a factor in our computational pipeline described below.

### Example 3

Evolutionarily conserved transcriptome response to N-treatment used for feature reduction in machine learning

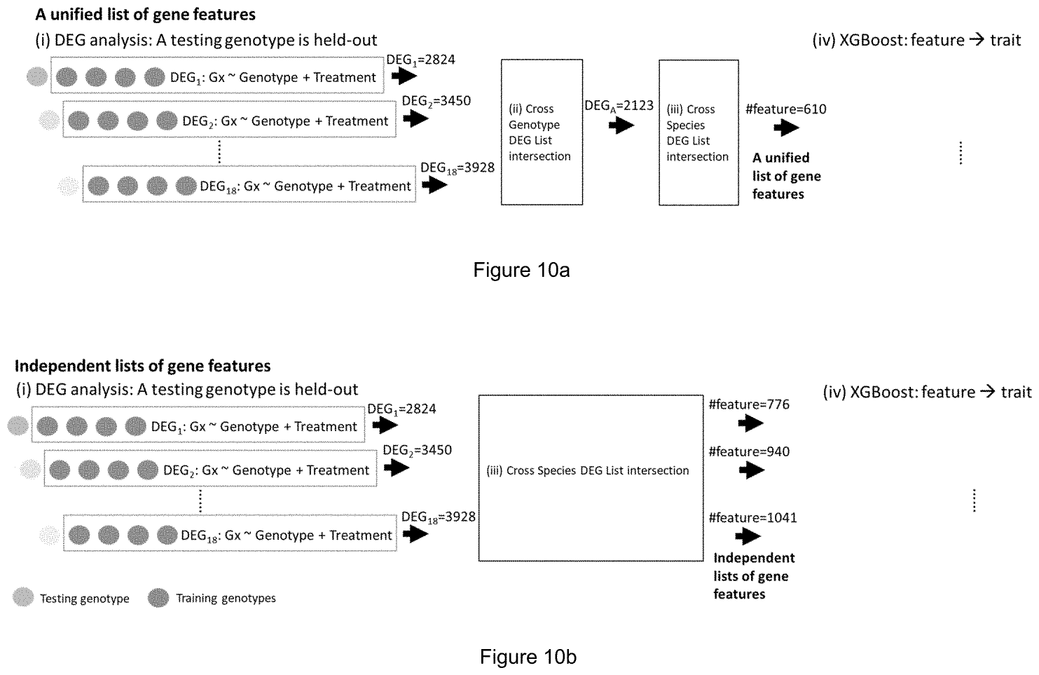

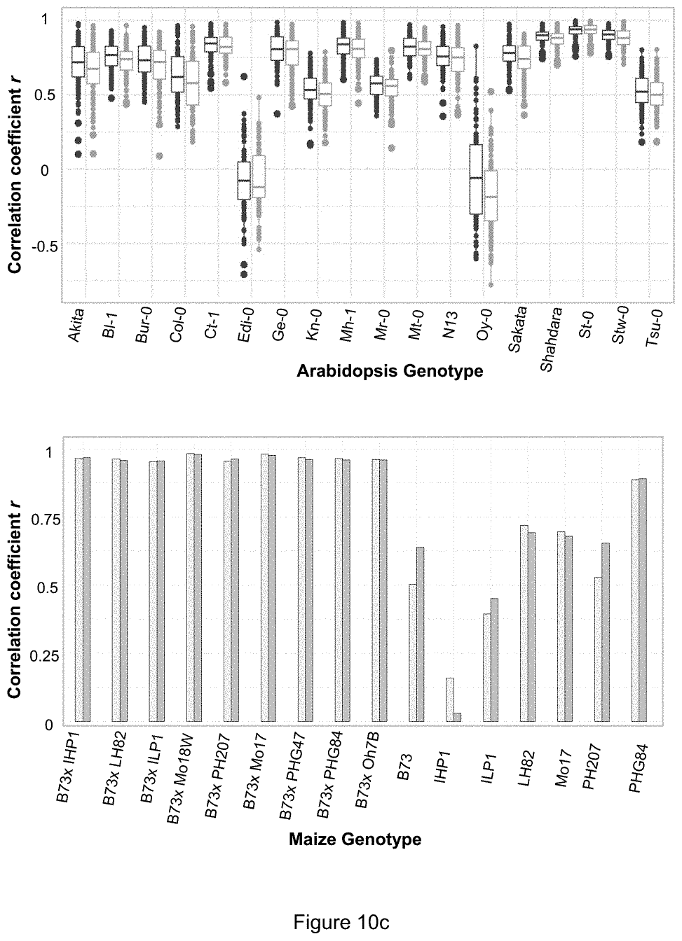

Feature reduction is an essential pre-processing step in machine learning, as too many irrelevant features may interfere with prediction performance³. Given the fact that the N level is a significant factor explaining NUE variation in both Arabidopsis and maize (FIGS. 2c and 3c), we used negative binomial Generalized Linear Mixed models (GLMs) in edgeR R-package¹⁷ and identified N-DEGs (Gene expression˜Condition+Genotype) in the training data (n-1 genotype). Importantly, we note that the testing data sets (the held-out genotype) were never used to select the N-DEGs. This was repeated in a round-robin manner across genotypes for each species (FIG. 10). Next, we retained the evolutionarily conserved N-DEGs by mapping the Arabidopsis N-DEGs to their corresponding maize homologs using Phytozome 10¹⁸ (FIG. 4). This cross-species analysis enabled us to i) apply an evolutionarily guided filter to reduce the dimensionality of gene features used in machine learning, and ii) enhance our ability to perform rapid validation testing of candidate NUE genes with relevance to the crop in the model species.

The resulting conserved N-DEGs from Arabidopsis (n=610) were used as gene features in the machine learning model (FIG. 5). We further subjected the conserved N-DEGs from maize to a second round of filtering to identify those also responding to N×G interaction (FIG. 4, Within-species Feature Reduction). This second filter aimed to account for the significant N×G effect that we observed in the maize NUE phenotypes (FIG. 3c), resulted in a list of maize N-DEGs responsive to N×G interaction (n=248). Next, these two sets of conserved N-DEGs from Arabidopsis and maize were used as features in the machine learning model (FIG. 5).

We then analyzed whether the expression levels of N-DEGs conserved across model and crop species could enhance identification of NUE phenotypes—compared to non-selected genes—using machine learning algorithms. This data-driven hypothesis is supported by the fact that: i) the expression levels of N-DEGs have been used as biomarkers of N status across maize genotypes¹⁹, and ii) the described phenotypic data shows that N level is a significant factor explaining the NUE variation in both maize and Arabidopsis (FIGS. 2c and 3c). Indeed, this analysis enabled determining that the predictive performance of the described models is significantly better at predicting NUE outcomes when the evolutionarily conserved N-DEGs are used, compared to the same number of top-ranked N-DEGs with the lowest P-value, or randomly selected expressed genes (Table 1), as detailed below.

### Example 4

Evolutionarily Conserved N-Responsive Genes have Enhanced Predictive Power in Machine Learning

For each species, we used the gene expression values (N-DEGs) as features (also referred to as gene features) to predict NUE traits through XGBoost regression models. XGBoost¹³ is a implementation of the gradient boosting algorithm²⁰, that uses a boosting algorithm to combine multiple weak learners, i.e. shallow trees, into a strong one (FIG. 5, Step 2). Lastly, we used the trained XGBoost models to predict NUE for the left-out genotype and evaluated the model performance using correlation between the observed- and the predicted-NUE in the left-out test set (FIG. 5, Step 3). In summary, we repeated the above steps and constructed 18 models for Arabidopsis, and 16 models for maize, corresponding to each genotype analyzed (See FIG. 10 for an illustration).

For maize, using the N-DEGs (n=248) conserved with their Arabidopsis homologs, resulted in a mean Pearson's correlation coefficient r of 0.79 for the XGBoost models predicting NUE across 16 maize lines (FIG. 5, Step 3). The r was above 0.6 for all but two maize genotypes, Illinois High Protein (IHP1) and Illinois Low Protein (ILP1). These two maize inbred line are derived from more than 100 cycles of divergent selection for seed protein concentration and other component traits of nitrogen use efficiency^{21,22}. The models showed lower accuracy in predicting the NUE phenotypes of IHP1 and ILP1, compared to other maize inbreds and the hybrids that each share the B73 parent.

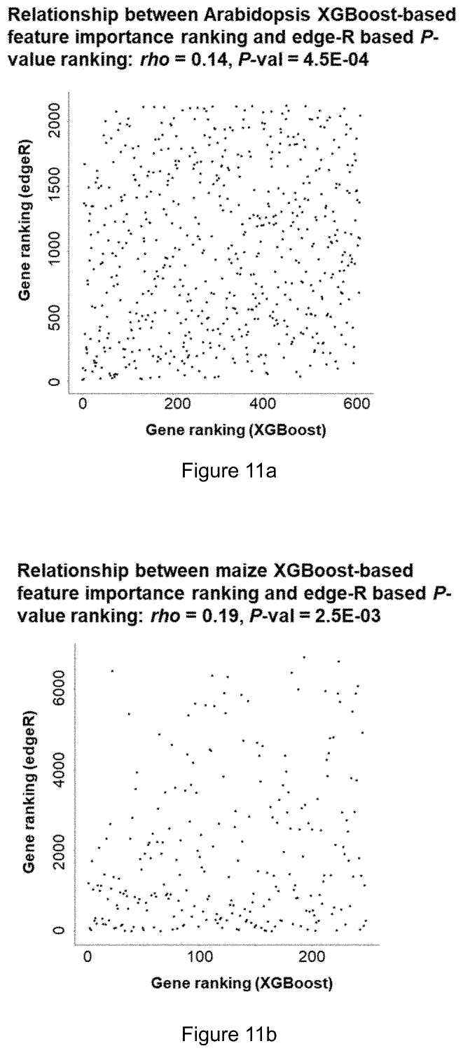

The described analysis showed that the overall predictive performance of learned models that used the evolutionarily conserved maize N-DEGs is significantly better than that obtained using the same number of top-ranked N-DEGs with the lowest P-value (0.68, Mann-Whitney U test P-value=1.06E-3), or ones randomly selected from total expressed genes (0.62, Mann-Whitney U test, P-value=1.5E-5) (Table 1). In addition, comparison of the feature importance score, an XGBoost¹³ output which reveals the influence of each feature (gene) in the predicted value (NUE)¹³, with the P-value in DEG analysis, uncovered only a weak correlation (Spearman's rank correlation coefficient rho=0.19, FIG. 11b). These comparisons support the interpretation that XGBoost models capture non-linear gene-trait relationships and our hypothesis that evolutionarily conserved N-DEGs enhance the machine learning outcome.

In parallel, we used the Arabidopsis N-DEGs (n=610) whose N-response is conserved with their maize homologs, as the features to predict NUE in the same XGBoost machine learning pipeline (FIG. 5). Our machine learning results show that the mean Pearson's correlation coefficient r across all 18 Arabidopsis genotypes was 0.65 (FIG. 5, Step 3). Moreover, we found that this overall model performance is significantly better than that obtained using the same number of top-ranked N-DEGs with the lowest P-value (r=0.59, Mann-Whitney U test P-value=1.64E-4), or ones randomly selected from total expressed genes (r=0.53, Mann-Whitney U test, P-value=3.82E-6) (Table 1). Similarly, we found that the feature importance ranking was weakly correlated with the edgeR-based P-value ranking of DEGs (Spearman's rank correlation coefficient rho=0.14, FIG. 11a).

The described results from both maize and Arabidopsis data show that using the evolutionarily conserved N-responsive differentially expressed genes significantly improved performance of the machine learning models predicting NUE significantly, and that this improvement is not due to a simple numerical reduction in the gene features (Table 1). Furthermore, the weak correlation between the XGBoost-based feature importance ranking and the edgeR-based P-value ranking (FIG. 11), indicates that XGBoost can capture non-linear gene-trait relationship beyond single variable DEG analysis. We used one set of hyperparameters for each species to achieve a consistent performance across genotypes, suggesting that the model is generalized and likely applicable to additional genotypes. Taken together, the results demonstrate that NUE—a polygenic trait—could be predicted from gene expression levels of N-DEGs, and that using an evolutionarily principled approach to feature reduction significantly improved the model performance.

### Example 5

### Predicting Additional Traits Demonstrates the General Applicability of the Evolutionarily Informed Machine Learning Pipeline

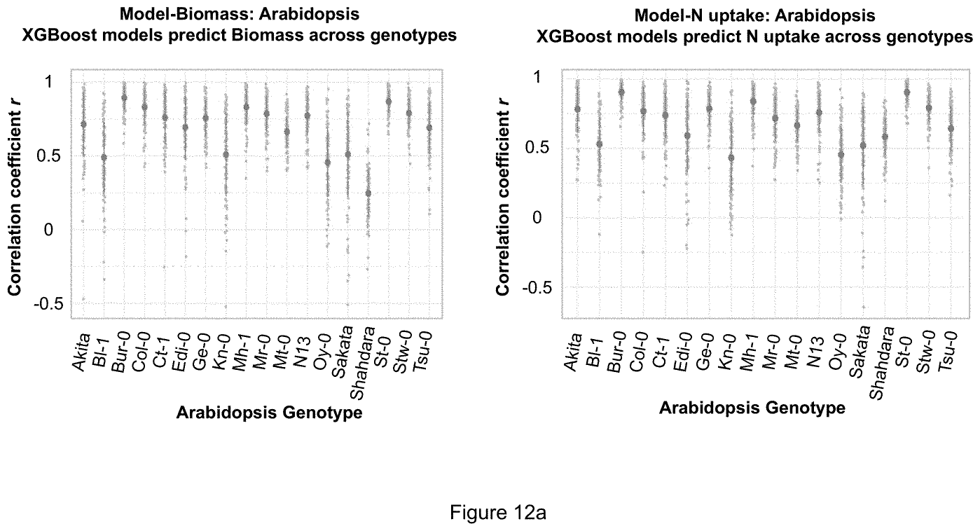

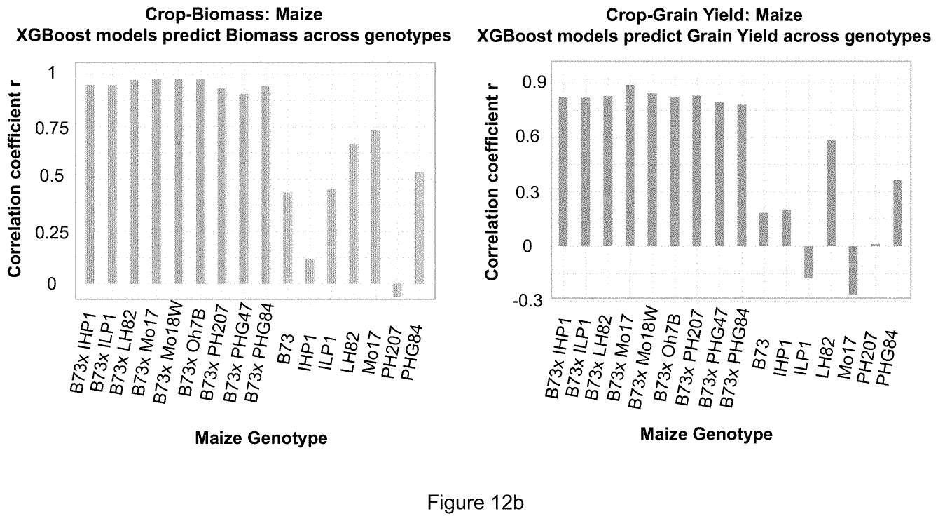

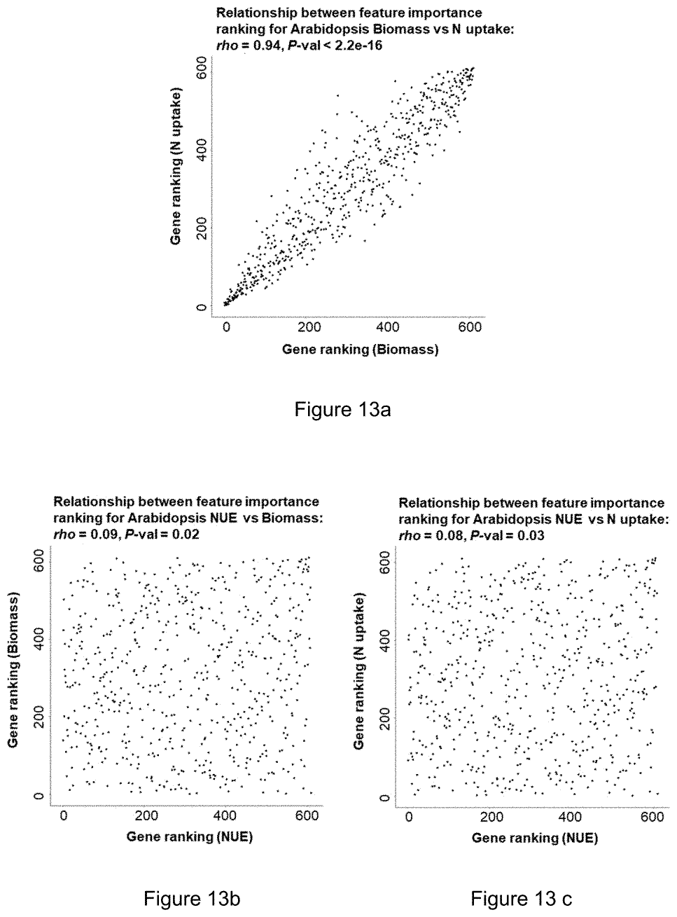

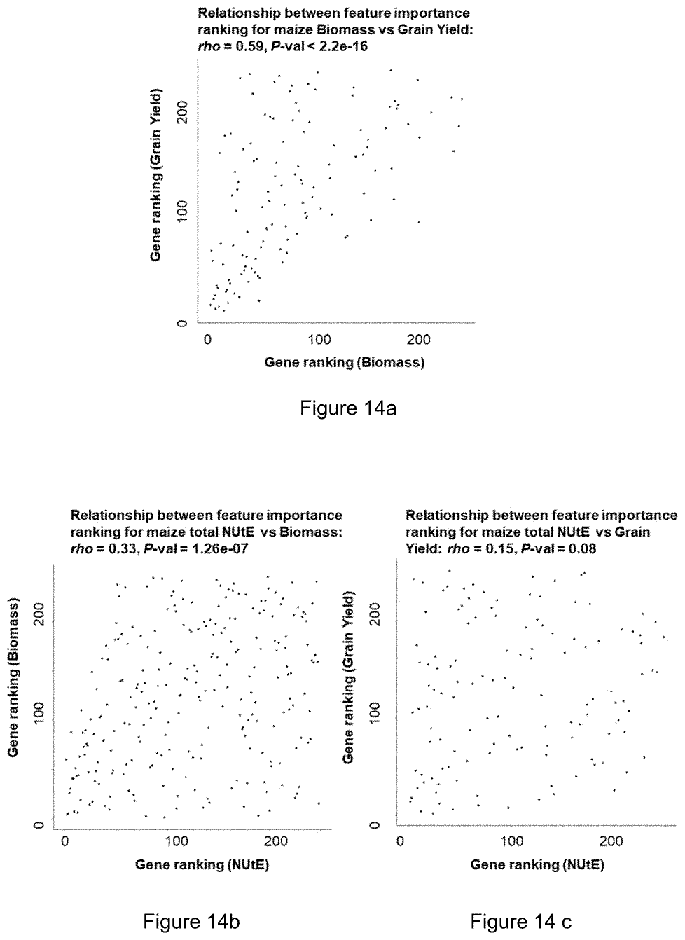

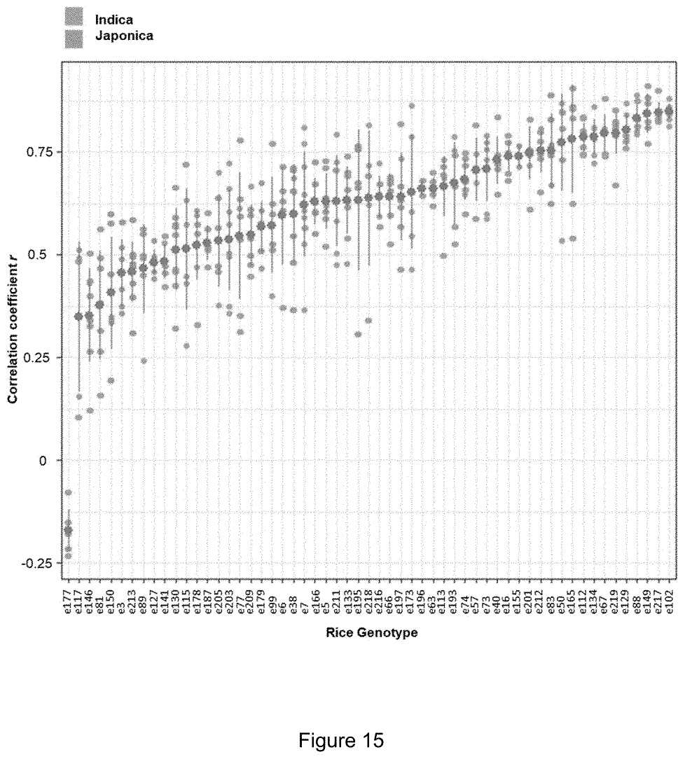

To further test whether our pipeline can be applied to predict additional traits from transcriptome data, we used the same conserved N-DEGs (FIG. 4), to predict two additional traits for each species. For Arabidopsis, we found that the mean Pearson's r for predicting biomass and N-uptake was 0.68 and 0.69, respectively (FIG. 12a), is comparable to that for predicting NUE (r=0.65). The feature importance ranking appeared to be trait-specific, as the gene ranking for NUE only weakly correlated with those for biomass (rho=0.09) and N-uptake (rho=0.08) (FIG. 13b, 12c). This result can be explained by the weak correlation between NUE and biomass (r=0.14), as well as that between NUE and N-uptake (r=0.01) (FIG. 2b). For highly correlated traits such as biomass and N-uptake (r=0.97), the feature importance rankings were also highly correlated (rho=0.94) (FIG. 13a). For maize, the mean Pearson's r for predicting biomass and grain yield was 0.72 and 0.52, respectively (FIG. 12b). As with Arabidopsis, the feature importance rankings for maize also appeared to be trait-specific, being greater (rho=0.59) for highly correlated traits such as biomass and grain yield (r=0.8), compared to Total NUtE—which is weakly correlated with either biomass (r=−0.14; rho=0.15) or grain yield (r=−0.19; rho=0.33) (FIG. 3b, FIG. 14). Taken together, these results indicate that the feature importance ranking can capture biological information represented by the degree of phenotypic correlation among different component traits.

We also applied the described evolutionarily informed machine learning pipeline to two additional matched transcriptome and phenotype datasets related to drought in field grown rice and disease response in mouse models.

The rice data comprises matched transcriptomic and phenotypic information collected from 220 rice genotypes subjected to drought treatment in field experiments²³. The 220 rice genotypes consist of two major subspecies, Indica and Japonica, which diverged ˜440,000 years ago, with the genotypic and phenotypic diversity of domesticated rice. From this large dataset, we retained 57 rice genotypes that had no missing data in the trait measurement. We then used this set of 57 rice genotypes, and randomly selected 20 genotypes to define drought-responsive DEGs and used them as gene features for predicting the fecundity in the 37 “left-out” rice genotypes. We repeated this process 10-times and the mean Pearson's r was 0.62. The model performance was consistent across the evolutionarily distant Japonica and Indica rice sub-species (FIG. 15), and better than using the same number of random expressed genes (Mann-Whitney U test, P-value <2.2e-16).

The mouse dataset comes from a highly genetically diverse Collaborative Cross (CC) population that comprises 90% of the genetic diversity across the entire laboratory Mus musculus genome²⁴. The dataset we selected comprises matched transcriptome and disease outcome after influenza virus infection of 11 genotypes from the CC mouse population study²⁴. We used DEGs (mock vs. infected) identified across the 11 mouse CC population genotypes to predict the disease outcome (asymptomatic vs. symptomatic) and found the mean Pearson's r to be 0.98. The models built using cross-genotype DEGs outperformed the model using the same number of random expressed genes (Mann-Whitney U test, P-value=3.3E-3).

Overall, the results for the matched transcriptome and phenotype datasets for the rice and mice models provide two use-case studies of evolutionarily informed machine learning pipeline applied to external data sets for traits in both plants and animals. They also show that transcript-based prediction can be achieved using a smaller population (20 and 11 genotypes in the case of rice and mice respectively), compared with the requirement of hundreds of lines which are needed for GWAS and eQTL studies²⁵.

### Example 6

### Validating the Function of Genes Whose Expression is Influential in Models Predicting NUE

The Examples above established the robustness of the evolutionarily informed machine learning models in predicting trait outcomes based on conserved gene responses within and across species. Next, we experimentally validated gene features that are most influential in our predictive models. To this end, we used the feature importance score, an XGBoost¹³ output which reveals the influence of each feature (gene) in the predicted value (NUE). We reasoned that if models built for multiple genotypes selected a common set of gene features, this would indicate that those gene features are robust to genotype in predicting NUE. In maize, over 81% (202/248) of the XGBoost “important gene features” for predicting NUE were shared by models built for 16 genotypes, and 91% (245/248) were shared by 10 or more maize genotypes. Similarly, for Arabidopsis 42% (257/610) of the “important features” for predicting NUE were shared by models built for 18 Arabidopsis accessions, and 85% (519/610) were shared by 10 or more Arabidopsis accessions. These results are not only consistent with the polygenic nature of NUE trait, but also reveal that there is a core set of influential N-DEGs whose expression levels can accurately predict NUE phenotypes for both species.

In maize, the top-ranked “important gene features” in predicting NUE outcomes include the transcription factors (NLP, MYB, WRKY), members of N-uptake/assimilation pathway (ammonium transporter, asparagine synthetase), and genes involved in photosynthesis and amino acid metabolism (FIG. 5, Step 4,). In Arabidopsis, the top-ranked “important gene features” in predicting NUE include transcription factors (NF-Y, NLP, MYB), members of the N-uptake/assimilation pathway (nitrate transporter, asparagine synthetase, glutamine synthetase), tubulins, and chlorophyll a-b binding proteins (FIG. 5, Step 4). Several of the important features including the transcription factors (NLPs, LBD37/LBD38) and genes involved in N-metabolism (glutamine and asparagine synthetase) have been implied or directly linked to affect NUE in planta^{19,26-29}. This consistency of our machine learning predictions of genes of “importance” to NUE with published results in planta not only validates the findings from the described machine learning pipeline, but also indicates the novel genes uncovered in this pipeline can shed light on additional previously unknown molecular components and mechanisms underlying NUE.

Further, we reasoned TFs controlling the levels of expression of multiple XGBoost important features for predicting NUE would be candidates for functional validation for their role in NUE in planta. To this end, we identified TFs predicted to regulate these XGBoost gene features of importance to NUE by constructing gene regulatory networks (GRNs) using GENIE3, which adopts the random forest machine learning algorithm and was the best performer in the DREAM4 and −5 Network Inference Challenge¹⁴.

To construct GRNs controlling NUE for each species, we first identified the N-responsive TFs in maize (545 TFs) and Arabidopsis (184 TFs) by intersecting the N-DEGs in this disclosure with the TFs for each species using published databases³⁰⁻³². Next, we used our N-response TFs in GENIE3 as the “regulatory genes” (GENIE3 term) whose influence on the evolutionarily conserved “target genes” in maize (248 gene features) or Arabidopsis (610 gene features) were weighed on a 0 to 1 scale, where 0=non-influential and 1=strongly influential. We kept the top 1% of the TF-target edges to construct the NUE regulatory network and calculated the number of TF-target edges (connectivity) for each TF as a measure to evaluate their influence within the GRN.

Next, we integrated our GRN analysis with the XGBoost results to select candidate TFs that regulate genes of importance to NUE phenotype for functional validation of their role in NUE (Table 2). The selection and prioritization of TFs was based on one or more of the following criteria: i) XGBoost-based importance score, ii) GENIE3-based TF connectivity in the NUE GRN, iii) curated knowledge from the literature, and iv) the availability of multiple mutant alleles. In Arabidopsis, the top TFs in the XGBoost-based importance ranking listed in Table 2 include NF-YA6 (AT3G14020), D1V1 (AT5G58900), UNE12 (AT4G02590), NLP5 (AT1G76350), and TCP2 (AT4G18390). The other two Arabidopsis TFs prioritized for in planta validation studies WRKY38 (AT5G22570) and WRKY50 (AT5G26170) (Table 2), were selected based on their high connectivity in the GENIE3-based GRN. For maize, we selected two candidate TFs (Zm00001d006293 nlp17, Zm00001d012544 myb74) for in planta validation studies that are hubs in the GENIE3-based GRN. Since no maize mutants were available for these genes, we took advantage of our cross-species approach by validating the function of their Arabidopsis homologs (AT1G76350 NLP5, AT5G06100 MY833) in NUE. With the goal of cross-species validation, we also selected the maize homolog (Zm00001d006835, nfya3) of the top-ranked Arabidopsis NF-YA6 (AT3G14020) for validation in NUE (Table 2). This choice took into consideration the fact that NF-Y transcription factors are enriched in Arabidopsis XGBoost gene features and in the maize GRN. Moreover, this selection was supported by previous studies which showed that overexpressing a member of the NF-YA family in wheat significantly increased N uptake and grain yield under different levels of N supply³³. To discern the function of maize NF-Y homologs in NUE, we characterized the nfya3-1::UfMu mutation with a Uniform Mu transposon insertion (mu1003041)³⁴ that does not produce a detectable full-length transcript.

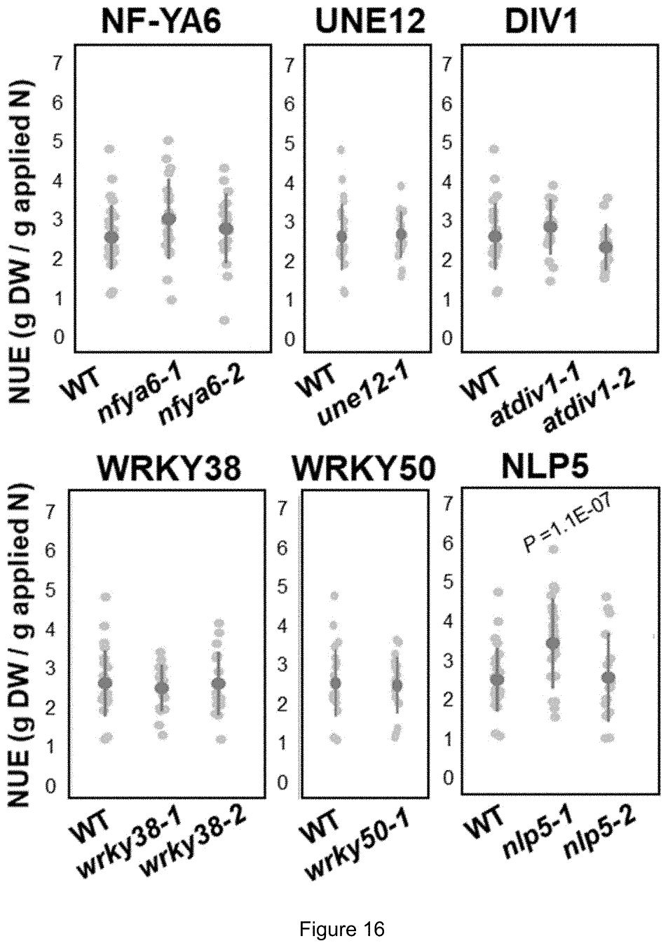

Our results on the eight Arabidopsis TFs selected for in planta validation studies were classified into two groups based on our NUE phenotypic results (FIG. 6). The Group I “important gene features” in predicting NUE in Arabidopsis include MY833 (AT5G06100) and TCP2 (AT4G18390), which when mutated showed increased NUE phenotypes under both high- and low-N inputs (FIG. 6a). These validation results reveal that each TF plays a non-redundant role as negative regulators of NUE, as the loss-of-function T-DNA mutants displayed higher NUE under both N-deplete and N-replete conditions. The Group II “important gene features” in Arabidopsis include 6 TFs which when mutated show increased NUE phenotypes specifically under low-N input: UNE12 (AT4G02590), NLP5 (AT1G76350), NF-YA6 (AT3G14020), WRKY38 (AT5G22570), WRKY50 (AT5G26170), and D1V1 (AT5G58900) (FIG. 6b). These validation results reveal that each of these Class II TFs plays a non-redundant role as negative regulators of NUE, as the loss-of-function T-DNA mutants displayed higher NUE, specifically under N-deplete conditions (FIG. 6b, FIG. 16), suggesting that the function of these TFs in regulating NUE is only required when N is limiting. Alternatively, their function may be redundant with other TFs under N-replete conditions. For maize, the NNUE tests of the nfya3-1::UfMu mutant in the field showed that they accumulated less stalk and total N compared to wild-type, yet grain biomass and all other traits dependent on grain biomass (grain yield, harvest index, NUtE) increased when grown with sufficient N (FIG. 6c). These results show that loss of maize NFYA3 influences how developing seeds sense and respond to plant N status, with the mutation reducing the N requirement to promote grain, thereby enhancing the NUtE. Observing phenotypes in the grain is also consistent with the expression pattern of NFYA3, which is strongest in developing seeds³⁵. No significant differences were observed for NUE traits compared to wild-type maize (W22) when grown under N-limiting conditions, except for slightly lower grain yield and higher grain N concentration.

Taken together, the described evolutionarily informed machine learning predictions of genes of importance to NUE and validation results for TF mutants for both Arabidopsis and maize demonstrate that: i) Using evolutionarily conserved gene response significantly enhances the ability of the XGBoost machine learning models to predict NUE outcome across genotypes and species (plants and animals), and ii) The XGBoost-based important scores and GENIE3-based connectivity are informative in selecting functionally important features—including TFs—to control of a complex physiological trait in crops— NUE—which has important implications for sustainable agriculture.

It will be recognized from the foregoing Examples that the disclosure described a new genome-to-phenome analysis—namely, predicting phenotypic outcomes from genome-wide expression data. We show that exploiting evolutionary conserved gene expression datasets—within and across species—enhanced the machine learning model performance in predicting NUE phenotypes in a model (Arabidopsis) and a crop (maize), and also as applied to published matched transcriptome/phenotype datasets from another crop (rice) and model animal (mouse).

Our evolutionarily informed three-step machine learning pipeline (FIG. 1) which integrates phenotypic traits, transcriptome profiles, genetic variation, and environmental responses allowed us to; 1) preselect a subset of transcripts based on an evolutionarily conserved transcriptome responses within and across species, 2) employ this conservation as a biologically-principled way to reduce the feature dimensionality to improve the machine learning mmodel performance, and 3) rapidly validate the function of ‘important gene features’ identified from XGBoost models and GENIE3 gene regulatory network via the inclusion of a model and crop species.

The implementation of machine learning in predicting phenotypes has advanced in the past few years. However, the available datasets do not always; 1) exploit the genetic diversity of the organism(s) and 2) measure the phenotypes using same samples from which the transcriptome response was captured. The present disclosure advances the field in both points, as we utilized a panel of genotypes with diverse genetic backgrounds and measured phenotypes from the same batch of plants that the transcriptome was captured. We integrated genetic diversity, machine learning, and cross-species approaches to identify genes of importance to an agronomically important trait, NUE. The trait we selected for study on NUE has the challenge of its underlying polygenic nature and the difficulty in collecting high quality phenotypic data³⁶. To this end, we designed a sufficiently large experimental space of N-treatments across a set to ˜20 genotypes spanning NUE phenotypes in a model and crop species. The described results represent the largest matched phenotypic and transcriptomic datasets from both a model and a crop species. This dataset includes a large NUE phenotypic dataset resource of 318 maize genotypes for the plant community, and for 18 Arabidopsis accessions. We analyzed the genetic diversity in 18 Arabidopsis accessions and 23 maize genotypes selected for broad phenotypic variation in NUE and scored them for both transcriptomic and physiological responses in the same samples. Importantly, the selected maize genotypes represent the range of NUE diversity observed among a comprehensive collection of germplasm adapted to the U.S. Corn Belt, as confirmed empirically (FIG. 8).

To extend this analysis beyond NUE, we applied our evolutionarily informed machine learning approach to other agricultural traits (e.g. drought resistance) in another major crop, using published transcriptome and phenotype datasets of genetically diverse rice subspecies (Indica and Japonica)²³. In our application to animals, we exploited the growing awareness that host genetic variation has a major impact on pathogen susceptibility. To this end, we used matched transcriptome and phenotype data from a highly genetically diverse Collaborative Cross (CC) population that comprises 90% of the genetic diversity across the entire laboratory Mus musculus genome²⁴. Models that we built using cross-genotype DEGs from both these studies of these genetically diverse lines in plants (rice) and animals (mice) lines, significantly outperformed the model using the same number of random expressed genes. Importantly, in these two additional case studies, and in our proof-of-principle example, our evolutionary informed analysis of matched transcriptome and phenome data allowed us to use a considerably smaller sample size compared to those needed for GWAS or eQTL studies²⁵.

By providing accurate prediction, the predictive models reveal novel gene features for further investigation of causality³⁷. We demonstrate this principle using a reverse genetics approach to validate the function of eight transcription factors important to predicting NUE outcomes (Table 2). Notably, our two-way cross-species validation strategy enabled us to verify the function of genes involved in NUE for i) two maize candidate genes using mutants in their Arabidopsis homologs and ii) one Arabidopsis candidate TF via analysis of a mutant in its maize homolog grown in the field (Table 2, FIG. 6).

The learned model performance is more robust to maize genotype, compared with the models learned in Arabidopsis (FIG. 5). This outcome was obtained even though the maize genotypes used in the Examples possess greater genetic diversity of NUE (FIG. 3c). Many factors may contribute to this difference. For instance, the maize gene features were applied to forecast NUE traits measured at later development stages (FIG. 7). By contrast, the Arabidopsis gene features were applied to predict the NUE traits measured at the same time as RNA samples (FIG. 7).

The disclosure reveals that genes affecting NUE are involved in an array of processes (Table 2), including nutrient response and uptake (DIV1⁴⁰ and NLP5^{19,41}), anther and pollen development (NF-YA6⁴² and MYB33⁴³), juvenile-to-adult transition (MYB33⁴⁴), microRNA-mediated growth and responses (NF-YA⁴⁵, MYB33⁴⁴, TCP2⁴⁶), immune response (NF-YA6⁴², UNE12⁴⁷, WRKY38⁴⁸, and WRKY50⁴⁹), and photomorphogenesis (TCP2⁵⁰ and Zm00001d006835⁵¹). These results not only provide additional evidence supporting the notion that NUE is a polygenic trait and intertwined with diverse signaling pathways, but further reveal a novel role of these genes in regulating NUE. Notably, there are three transcription factor families, NF-Y, NLP, and WRKY, whose members are enriched as the gene features of XGBoost models and/or the regulators of GENIE3-based GRN.

Our results identified nine Arabidopsis and one maize NF-Y genes as the features in XGBoost models, as well as 12 Arabidopsis and 14 maize NF-Y genes, as potential regulators in the GENIE3 NUE GRN. Moreover, we validated the function of NF-YA6 in NUE—a top gene in Arabidopsis XGBoost model —using mutants in Arabidopsis NF-YA6 (AT3G14020), as well as its maize homolog nfya3 (FIG. 6) and expect similar results by inhibiting expression of hb7. The NF-Y family, found in nearly all eukaryotes⁵², encodes components of an evolutionarily conserved trimeric transcription factor complex. In humans, NF-Y binds to the CCAAT box in promoters of large sets of genes overexpressed in breast, colon, thyroid, and prostate cancer⁵³. In plants, the regulatory roles of NF-Y have been revealed in flowering-time, early seed development, nodulation, hormone signaling, and stress responses⁵². NF-Ys function as a multimeric protein complex (NF-YA/B/C(-CO/bZIP/bHLH) to bind its canonical motif CCAAT and/or the motif(s) of its partner TFs⁵⁴. It is possible that the flexible cis-binding capacity makes NF-Ys versatile and context-dependent TFs that can quickly adapt to nutrient fluctuations. It is noteworthy that several NF-Y genes are targeted and down-regulated by miR169⁵⁵ and miR169 members respond transcriptionally to N-starvation⁵⁶. Thus, the disclosure supports a new link between N-signaling, miRNA changes in N-responsive of NF-Ys, to the phenotypic output of NUE: Nitrogen→miR169→NF-Y→NUE.

We identified six Arabidopsis and two maize NLP genes as the features in XGBoost models to predict NUE, as well as five Arabidopsis and 14 NLP genes as potential regulators in the GENIE3 NUE GRN. Further, using mutants, we validated the role of NLP5—a top gene feature in maize XGBoost model and maize NUE GRN—as a negative regulator of NUE specifically under low-N conditions (FIG. 6b, FIG. 15). The NLPs—which are plant-specific TFs—are related to a core symbiotic gene Nin⁵⁷ and later identified as master regulators of nitrate signaling in Arabidopsis²⁶. Emerging evidence suggests their contribution to N-regulated gene expression and developmental processes is common across plant species⁵⁸. The results from our functional validation experiment indicated that NLP5 is a negative regulator of NUE under N-depleted conditions (FIG. 6B), which can be explained by the fact that NLP5 is a target of NIGT1/HRS1, a master regulator of N-starvation response genes^{59,60}. Thus, the loss of NLP5 in the Arabidopsis mutants could de-repress the N-starvation response, leading to higher NUE.

We identified six Arabidopsis and six maize WRKY genes as the features in XGBoost models, as well as 24 Arabidopsis and 11 WRKYgenes as the regulators in GENIE3 NUE GRN. Among them, WRKY38 and WRKY50 are the top-ranked TF hubs in the Arabidopsis NUE GRN. Our functional analysis using Arabidopsis mutants validated a role of WRKY38 and WRKY50 in mediating NUE (FIG. 6B). WRKY5, occurring primarily in plants⁶¹, are among the largest families of transcription factors. Cumulative evidence has demonstrated the important biological functions of WRKY5 in plant developmental processes (embryogenesis, germination, senescence etc.) as well as response to biotic and abiotic stresses including defense, salt, drought, nutrient starvation and more⁶². In addition to their known functions in defense responses^{48,49}, our results add a novel aspect to WRKY38 and WRK50 in regulating NUE and make them candidate TF hubs in coordinating plant responses to N levels as well as biotic stress.

The disclosure demonstrates that the integration of genetic diversity, cross-species transcriptome analysis and machine learning method enhances predictive modeling of genes affecting NUE. The results from reverse genetic analysis further show that those genes predictive of NUE are not only biomarkers but are functionally important in determining plant performance in response to environmental nutrition. The pipeline described herein could complement current approaches in identifying important genes in a multigenic trait. Our validation of the evolutionarily informed strategy for feature reduction across both genetically diverse crop and animal datasets, supports its potential to inform any system that seeks to uncover important genes controlling a complex phenotype in biology, agriculture, or medicine.

### Example 7

This Example describes the materials and methods used to produce the described results.

### Plant Materials, Growth Conditions, and Phenotypic Assays

### Arabidopsis

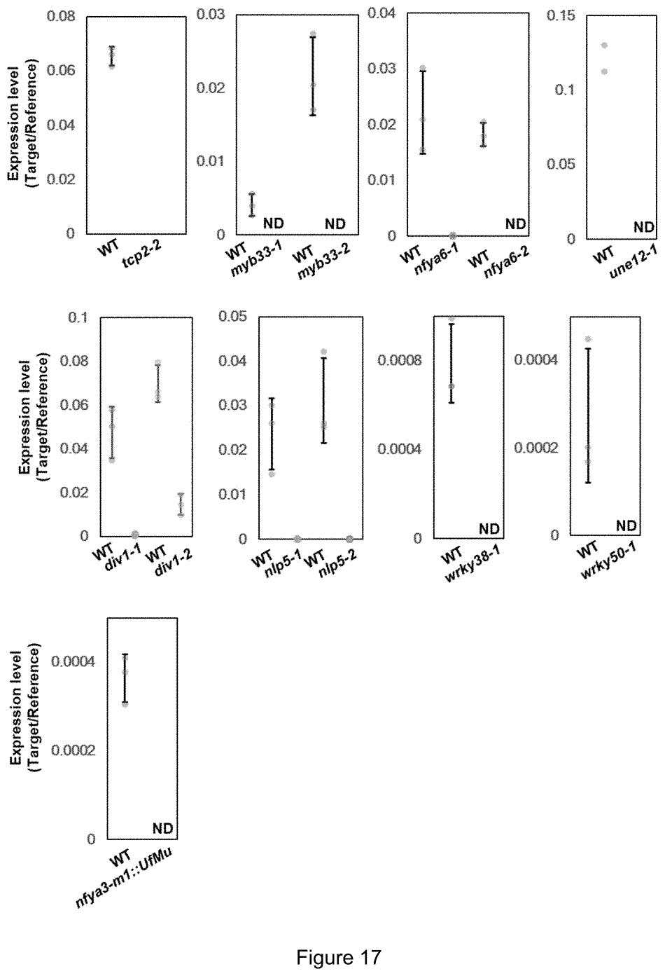

All Arabidopsis seeds used in this disclosure were obtained from ABRC. The 18 Arabidopsis accessions are Akita, B1-1, Bur-0, Col-0, Ct-1, Edi-0, Ge-0, Kn-0, Mh-1, Mr-0, Mt-0, N13, Oy-0, Sakata, Shandara, St-0, Stw-0, and Tsu-0, as previously studied for NUE⁸. The T-DNA mutants are all in the Col-0 background. The mutant lines⁶³ are myb33-1 (SALK_056201), myb33-2 (SALK_065473), tcp2-2 (SALK_060818), une12-1 (SAILseq_711_E09.1), n1p5-1 (SALK_055211), n1p5-2 (SALK_063304), nfya6-1 (SALK_005942), nfya6-2 (SAIL_159_E03), wrky38-1 (WiscDsLox489-492C21), wrky38-3 (SAIL_749_B02), wrky50-1 (SAIL_115_C10), div1-1 (SALK_056735), and div1-2 (SALK_084867C). The mutants were genotyped to confirm the homozygosity. The expression level of the inserted gene in the homozygous mutants were below detection limit of real-time PCR (FIG. 17).

For growth experiments, the Arabidopsis seeds were germinated on ½ MS with MES Buffer and Vitamins (RPI cat M70800) plates for 7-10 days in on a 16h-light/8h-dark photoperiod. The seedlings were then transferred to pre-washed nutrient-poor matrix vermiculite under an 8 h light (120/μmol2/s)/16 h dark diurnal cycle, at temperatures 22 and 20° C. respectively and 40% humidity. We kept one plant per pot and carried out the entire experiment using Arasystem (https://www.arasystem.com/). To track the N supply for each plant, we treated each plant with the same amount of low N (LN, 2 mM KNO₃) (Sigma cat P6083) or high N (HN, 10 mM KNO₃) medium (Caisson Labs cat. no. MSP10) using a syringe and recorded the volume. The potassium concentration was maintained by supplementing KCl (Sigma cat P9333) to the LN medium. On 40 and 42 DAS, the treatment was enriched with 10% atom excess ¹⁵N for ¹⁵N influx analysis. To minimize the variation due to pot location in the growth chambers, the HN row was located adjacent to the LN row, and the flats were shuffled three times weekly. We repeated these experiments three times consecutively to obtain biological replicates for phenotypic and transcriptomic samples. For each of the 18 Arabidopsis accessions, mature leaves were harvested for transcriptome and the above ground tissues for physiological traits at 43 DAS. The dried tissues were ground and analyzed for total nitrogen using a PDZ Europa ANCA-GSL elemental analyzer interfaced to a PDZ Europa 20-20 isotope ratio mass spectrometer at UC Davis Stable Isotope Facility.

### Maize

Seeds for all maize inbreds used in this disclosure were originally obtained from the USDA-ARS North Central Plant Introduction Station in Ames, Iowa, except for the inbreds derived from the Illinois Selection Experiment and FR1064 as described in Uribelarrea et al²². Inbred lines were subsequently increased by controlled self-pollination, and hybrid seed produced by controlled crosses. We grew the maize plants in N-managed field plots in Urbana, Ill. between May and September in 2014-2016. The soil type is a Drummer silty clay loam, pH 6.2, that received either 200 kg/Ha fertilizer N or no exogenous applied N when the plants reached the V3 growth stage. Subsequent soil testing and measures of plant N recovery estimate approximately 60 kg N/ha were made available from the soil alone. The N fertilizer was applied as granular ammonium sulfate banded adjacent to plants at the soil surface. Plants were grown in a split-plot design where individuals in each main plot (2 rows 5.3 m long, 76 cm row spacing) were paired in adjacent rows of N-replete or N depleted condition to a final density of 49,000 plants per hectare for inbreds and 77,000 plants per hectare for hybrids. Genotypes within main plots were arranged by relative maturity to minimize its impact on NUE traits. Plots were maintained weed free by a pre-plant application of herbicide (atrazine+metalochlor) followed by hand weeding as needed.

Maize phenotyping was performed at the R6 growth stage, when plants have reached physiological maturity, but may not yet have fully senesced. Five plants from each plot were cut at ground level, ears removed, and a fresh weight obtained on the entire remaining plant material (stover, comprising mostly stalk by weight, followed by leaves, tassels, and husks). The stover was then shredded in a Vermeer wood chipper, a subsample was collected into a tared cloth bag, and the subsample fresh weight was recorded. Stover samples were oven-dried to dryness at least three days at 65° C. and the subsample dry weight used to estimate stover biomass. The dried stover was further ground in a Wiley mill to pass through a 2 mm screen, and approximately 100 mg used to estimate total nitrogen concentration by combustion analysis with a Fisons EA-1108 N elemental analyzer. Grain samples were dried for approximately one week at 37° C., after which grain was shelled from the cobs, and the cob weight recorded. The moisture content and N concentration within each 5-plant grain sample was estimated using near-infrared reflectance spectroscopy on a Perten DA7200 analyzer, using a custom calibration built with samples possessing a broad range of variation in composition and color. The nitrogen concentration calibration was established using data from total combustion analysis of grain samples as described above for stover.

The nfya3-1::Mu loss-of-function allele was generated by the UniformMu insertion mu1003041::Mu in the 5′ untranslated region the annotated gene model Zm00001d006835. The UFMu-00332 seed stock was obtained from the Maize Genetics Cooperation Stock Center and genotyped⁶⁴ to identify homozygous for the nfya3-1::Mu mutant allele, which were then self-pollinated. The expression level of the nfya3 gene in the homozygous mutants was below detection limit of real-time PCR (CT>45) (FIG. 16). The nfya3 mutant and wildtype W22-Uniform Mu plants were grown in 2020 at the same field site and using the same experimental design, nitrogen treatments, and phenotyping methods described above.

### RNA Extraction, Library Preparation, and Sequencing

For each of three Arabidopsis RNA replicates, we harvested mature leaves from pre-bolting plants on 43 DAS between 9 and 11 AM from two plants, flash froze in liquid nitrogen and stored in −80 C. We isolated RNA using Direct-zol RNA Kits following manufacturer's instructions (Zymo Research). RNA quality was assessed on an Agilent Tape station using RNA ScreenTape (Agilent cat 5067-5576). All 108 stranded RNA-seq libraries were made using the NEBNext® Ultra™ II Directional RNA Library Prep Kit for Illumina® (NEB cat E7768) and assessed using DNA high sensitivity D1000 ScreenTape system (Agilent cat 5067-5584). The RNA-Seq libraries were sequenced using Illumina HiSeq 2500 v4 with 1×75 bp single-end read chemistry at the GenCore Facility at New York University Center for Genomics and Systems Biology.

For each of three maize RNA replicates, we collected leaf tissues from two inches from the base of leaf 13 subtending the top ear at R1 stage between 9 and 11 AM, flash froze in liquid nitrogen and stored in −80 C. We extracted RNA from frozen leaf tissue using CTAB-chloroform method. Genomic DNA was removed using DNAse I (NEB cat M0303). RNA-seq libraries were prepared using a TruSeq Stranded mRNAseq Sample Prep kit (Illumina cat RS-122-2101) according to the protocol provided. Single-end 150 bp reads were generated using the Illumina HiSeq 4000 at the Roy J Carver Biotechnology Center in the University of Illinois at Urbana-Champaign.

### Identification of N Response Differentially Expressed Genes (N-DEGs)

All RNA-seq raw reads were processed using the same pipeline to remove optical duplicates (Clumpify 37.24) and adapters (BBDuk 37.24)⁶⁵. The trimmed reads were aligned to the latest genome in 2018, TAIR10⁶⁶ for Arabidopsis and Zm-B73-REFERENCE-GRAMENE-4.0¹² for maize, using BBMap (37.24). The mapped reads were assigned by featureCounts (1.5.1)⁶⁷ using the latest annotation in 2018: Araport11⁶⁸ for Arabidopsis and AGPv4.32¹² for maize. The parameters and software versions for the above steps are available in GEO accession GSE152249. We identified N-DEGs in the training data set (n-1 genotypes) and repeated n times (n=number of genotypes in each species). In each round of analysis, we first filtered out the lowly expressed genes (CPM>1 in less than 10 samples) and normalized the data using upper-quantile (EDASeq 2.18.0)⁶⁹ and replicate samples (RUVSeq 1.18.0)⁷⁰. Subsequently, we used edgeR (3.26.8)¹⁷ to detect genes differentially expressed in high vs low N condition across genotypes (FDR <0.05). Lastly, we intersected the n lists of DEGs and only retained the ones occurring on n lists as a common set of N-DEGs. These analyses resulted in 2,123 Arabidopsis N-DEGs and 6,914 maize N-DEGs (FIG. 4). The Arabidopsis—Maize homolog mapping file is generated from Phytozome 10¹⁸.

We held out a testing genotype before the DEG stage; and only training genotypes (n-1 genotypes) were used in DEG analysis and XGBoost models. The held-out test genotypes were then used to validate the model performance. This round robin approach (FIGS. 10a(i) & 10b(i)), generated 18 and 16 independent DEG lists for Arabidopsis and maize, respectively. In approach a, we identified a unified list of gene features by intersecting these independent lists (e.g. 18 for Arabidopsis and 16 for maize) (FIG. 10a(ii)). By contrast, in approach b, cross species analysis was performed on each independent DEG list (e.g. 18 for Arabidopsis or 16 for maize).

To rule out the possibility that using the intersected DEGs (e.g. within species) would overly optimize the XGBoost results, we further compared the XGBoost performance using the intersected DEGs (FIG. 10a) with the alternative approach that did not go through the within species list intersection (FIG. 10b). The results of these two approaches are comparable (FIG. 10c). However, the advantage of conducting the cross-genotype intersection (FIG. 10a), which we used in this manuscript), has the benefit of resulting in a unified list of gene features, compared to multiple independent lists of gene features. Generating a unified list of gene features will enable the gene feature ranking across genotypes, rather than restricted to an individual genotype.

### Construction and Evaluation of Predictive Machine Learning Models

We used a tree model with gradient boosting, XGBoost¹³ R implementation, to train and test the models. For each species, we split the data into training (n-1 phenotypes) and testing (left-out genotype) sets. We used five-fold internal cross-validation to select the optimized hyperparameters. We tuned “nrounds” (number of trees), “colsample_bytree” (the proportion of features for constructing each tree), “subsamples” (the portion of training data samples for training each additional tree), and “eta” (shrinkage of feature weights to make the boosting process more conservative and prevent overfitting) in an XGBoost:regression model. Subsequently, we made predictions on each of the left-out genotype, assessed the model accuracy by calculating the Pearson's correlation coefficient r between the predicted and actual values⁷¹, and reported the r from 100 iterations.

### Selection of Candidate Genes for Functional Validation in NUE

We used two parallel procedures to select candidate genes for functional validation. First, we used the XGBoost-generated feature importance score that indicates how useful each feature was in the construction of model. We summed the score on a gene-by-gene basis from 18 models for Arabidopsis and 16 models for maize and generated a ranked list. Second, we used a Random Forest-based algorithm GENIE3 to infer the transcription factors regulating the gene features. We used the N-responsive TFs (184 Arabidopsis TFs and 545 maize TFs) as the regulators and the gene features (610 Arabidopsis genes and 248 maize genes) as the targets and kept the default parameters. We constructed the NUE regulatory network using the top 1% of the edges and ranked the TFs based on their connectivity (number of edges).

References—This reference listing is not an indication that any particular reference is material to patentability.
1 McMullen, M. D. et al. Genetic properties of the maize nested association mapping population. Science 325, 737-740, doi:10.1126/science.1174320 (2009).
2 Han, M., Okamoto, M., Beatty, P. H., Rothstein, S. J. & Good, A. G. The Genetics of Nitrogen Use Efficiency in Crop Plants. Annu Rev Genet 49, 269-289, doi:10.1146/annurev-genet-112414-055037 (2015).
3 Altman, N. & Krzywinski, M. The curse(s) of dimensionality. Nature Methods 15, 399-400, doi:10.1038/541592-018-0019-x (2018).
4 Burges, C. J. C. Dimension Reduction: A Guided Tour. Foundations and Trends® in Machine Learning 2, 275-365, doi:10.1561/2200000002 (2010).
5 Brubaker, D. K., Proctor, E. A., Haigis, K. M. & Lauffenburger, D. A. Computational translation of genomic responses from experimental model systems to humans. PLoS Comput Biol 15, e1006286, doi:10.1371/journal.pcbi.1006286 (2019).
6 Beatty, PH & Good, A. in Engineering Nitrogen Utilization in Crop Plants (eds Ashok Shrawat, Adel Zayed, & David A. Lightfoot) Ch. 2, 15-35 (Springer, 2018).
7 Zhang, X. et al. Managing nitrogen for sustainable development. Nature 528, 51-59, doi:10.1038/nature15743 (2015).
8 Chardon, F., Barthélémy, J., Daniel-Vedele, F. & Masclaux-Daubresse, C. Natural variation of nitrate uptake and nitrogen use efficiency in Arabidopsis thaliana cultivated with limiting and ample nitrogen supply. J Exp Bot 61, 2293-2302, doi:10.1093/jxb/erq059 (2010).
9 McKhann, H. I. et al. Nested core collections maximizing genetic diversity in Arabidopsis thaliana. Plant J 38, 193-202, doi:10.1111/j.1365-313X.2004.02034.x (2004).
10 Beckett, T. J., Morales, A. J., Koehler, K. L. & Rocheford, T. R. Genetic relatedness of previously Plant-Variety-Protected commercial maize inbreds. PLoS One 12, e0189277, doi:10.1371/journal.pone.0189277 (2017).
11 White, M. R., Mikel, M. A., de Leon, N. & Kaeppler, S. M. Diversity and heterotic patterns in North American proprietary dent maize germplasm. Crop Science 60, 100-114, doi:https://doi.org/10.1002/csc2.20050 (2020).
12 Jiao, Y. et al. Improved maize reference genome with single-molecule technologies. Nature 546, 524-527, doi:10.1038/nature22971 (2017).
13 Chen, T. & Guestrin, C. in Knowledge Discovery and Data Mining 10 (ACM New York, N. Y., USA, New York, N. Y., USA, 2016).
14 Huynh-Thu, V. A., Irrthum, A., Wehenkel, L. & Geurts, P. Inferring regulatory networks from expression data using tree-based methods. PLoS One 5, doi:10.1371/journal.pone.0012776
15 White, W. G., Vincent, M. L., Moose, S. P. & Below, F. E. The sugar, biomass and biofuel potential of temperate by tropical maize hybrids. GCB Bioenergy 4, 496-508, doi:10.1111/j.1757-1707.2012.01158.x (2012).
16 Haegele, J. W., Cook, K. A., Nichols, D. M. & Below, F. E. Changes in Nitrogen Use Traits Associated with Genetic Improvement for Grain Yield of Maize Hybrids Released in Different Decades. Crop Science 53, 1256-1268, doi:10.2135/cropsci2012.07.0429 (2013).
17 Robinson, M. D., McCarthy, D. J. & Smyth, G. K. edgeR: a Bioconductor package for differential expression analysis of digital gene expression data. Bioinformatics 26, 139-140, doi:10.1093/bioinformatics/btp616 (2010).
18 Goodstein, D. M. et al. Phytozome: a comparative platform for green plant genomics. Nucleic Acids Res 40, D1178-1186, doi:10.1093/nar/gkr944 (2012).
19 Yang, X. S. et al. Gene expression biomarkers provide sensitive indicators of in planta nitrogen status in maize. Plant Physiol 157, 1841-1852, doi:10.1104/pp. 111.187898 (2011).
20 Schapire, R. E. in Proceedings of the 16th international joint conference on Artificial intelligence—Volume 2 1401-1406 (Morgan Kaufmann Publishers Inc., Stockholm, Sweden, 1999).
21 Moose, S. P., Dudley, J. W. & Rocheford, T. R. Maize selection passes the century mark: a unique resource for 21st century genomics. Trends Plant Sci 9, 358-364, doi:10.1016/j.tplants.2004.05.005 (2004).
22 Uribelarrea, M., Below, F. E. & Moose, S. P. Grain Composition and Productivity of Maize Hybrids Derived from the Illinois Protein Strains in Response to Variable Nitrogen Supply. Crop Science 44, 1593-1600, doi:10.2135/cropsci2004.1593 (2004).
23 Groen, S. C. et al. The strength and pattern of natural selection on gene expression in rice. Nature 578, 572-576, doi:10.1038/s41586-020-1997-2 (2020).
24 Kollmus, H. et al. Of mice and men: the host response to influenza virus infection. Mamm Genome 29, 446-470, doi:10.1007/500335-018-9750-y (2018).
25 Korte, A. & Farlow, A. The advantages and limitations of trait analysis with GWAS: a review. Plant Methods 9, 29, doi:10.1186/1746-4811-9-29 (2013).
26 Konishi, M. & Yanagisawa, S. Arabidopsis NIN-like transcription factors have a central role in nitrate signalling. Nat Commun 4, 1617, doi:10.1038/ncomms2621 (2013).
27 Moison, M. et al. Three cytosolic glutamine synthetase isoforms localized in different-order veins act together for N remobilization and seed filling in Arabidopsis. J Exp Bot 69, 4379-4393, doi:10.1093/jxb/ery217 (2018).
28 Chen, Q. et al. Transcriptome sequencing reveals the roles of transcription factors in modulating genotype by nitrogen interaction in maize. Plant Cell Rep 34, 1761-1771, doi:10.1007/s00299-015-1822-9 (2015).
29 Yang, X. et al. QTL Mapping by Whole Genome Re-sequencing and Analysis of Candidate Genes for Nitrogen Use Efficiency in Rice. Front Plant Sci 8, 1634, doi:10.3389/fpls.2017.01634 (2017).
30 Yilmaz, A. et al. AGRIS: the Arabidopsis Gene Regulatory Information Server, an update. Nucleic Acids Res 39, D1118-1122, doi:10.1093/nar/gkq1120 (2011).
31 Jin, J. et al. PlantTFDB 4.0: toward a central hub for transcription factors and regulatory interactions in plants. Nucleic Acids Res 45, D1040-D1045, doi:10.1093/nar/gkw982 (2017).
32 Yilmaz, A. et al. GRASSIUS: a platform for comparative regulatory genomics across the grasses. Plant Physiol 149, 171-180, doi:10.1104/pp. 108.128579 (2009).
33 Qu, B. et al. A wheat CCAAT box-binding transcription factor increases the grain yield of wheat with less fertilizer input. Plant Physiol 167, 411-423, doi:10.1104/pp. 114.246959 (2015).
34 McCarty, D. R. et al. Steady-state transposon mutagenesis in inbred maize. Plant J 44, 52-61, doi:10.1111/j.1365-313X.2005.02509.x (2005).
35 Walley, J. W. et al. Integration of omic networks in a developmental atlas of maize. Science 353, 814-818, doi:10.1126/science.aag1125 (2016).
36 Myles, S. et al. Association mapping: critical considerations shift from genotyping to experimental design. Plant Cell 21, 2194-2202, doi:10.1105/tpc.109.068437 (2009).
37 Shmueli, G. To Explain or to Predict? Statistical Science 25 289-310, doi:10.2139/ssrn.1351252 (2010).
38 Breiman, L. Statistical Modeling: The Two Cultures (with comments and a rejoinder by the author). Statist. Sci. 16, 199-231, doi:10.1214/ss/1009213726 (2001).
39 Arp, J. J. Discovery of novel regulators and genes in nitrogen utilization pathways in maize Ph.D. thesis, University of Illinois at Urbana-Champaign, (2017).
40 Varala, K. et al. Temporal transcriptional logic of dynamic regulatory networks underlying nitrogen signaling and use in plants. Proc Natl Acad Sci USA 115, 6494-6499, doi:10.1073/pnas.1721487115 (2018).
41 Griffiths, M. et al. A multiple ion-uptake phenotyping platform reveals shared mechanisms affecting nutrient uptake by roots. Plant Physiol 185, 781-795, doi:10.1093/plphys/kiaa080 (2021).
42 Mu, J., Tan, H., Hong, S., Liang, Y. & Zuo, J. Arabidopsis transcription factor genes NF-YA1, 5, 6, and 9 play redundant roles in male gametogenesis, embryogenesis, and seed development. Mol Plant 6, 188-201, doi:10.1093/mp/sss061 (2013).
43 Millar, A. A. & Gubler, F. The Arabidopsis GAMYB-like genes, MYB33 and MYB65, are microRNA-regulated genes that redundantly facilitate anther development. Plant Cell 17, 705-721, doi:10.1105/tpc.104.027920 (2005).
44 Guo, C. et al. Repression of miR156 by miR159 Regulates the Timing of the Juvenile-to-Adult Transition in Arabidopsis. Plant Cell 29, 1293-1304, doi:10.1105/tpc.16.00975 (2017).
45 Sorin, C. et al. A miR169 isoform regulates specific NF-YA targets and root architecture in Arabidopsis. New Phytol 202, 1197-1211, doi:10.1111/nph.12735 (2014).
46 Palatnik, J. F. et al. Control of leaf morphogenesis by microRNAs. Nature 425, 257-263, doi:10.1038/nature01958 (2003).
47 Bruessow, F., Bautor, J., Hoffmann, G. & Parker, J. E. <em>Arabidopsis thaliana</em> natural variation in temperature-modulated immunity uncovers transcription factor UNE12 as a thermoresponsive regulator. bioRxiv, 768911, doi:10.1101/768911 (2019).
48 Kim, K. C., Lai, Z., Fan, B. & Chen, Z. Arabidopsis WRKY38 and WRKY62 transcription factors interact with histone deacetylase 19 in basal defense. Plant Cell 20, 2357-2371, doi:10.1105/tpc.107.055566 (2008).
49 Hussain, R. M. F., Sheikh, A. H., Haider, I., Quareshy, M. & Linthorst, H. J. M. Arabidopsis WRKY50 and TGA Transcription Factors Synergistically Activate Expression of. Front Plant Sci 9, 930, doi:10.3389/fpls.2018.00930 (2018).
50 He, Z., Zhao, X., Kong, F., Zuo, Z. & Liu, X. TCP2 positively regulates HY5/HYH and photomorphogenesis in Arabidopsis. J Exp Bot 67, 775-785, doi:10.1093/jxb/erv495 (2016).
51 Su, H. et al. Dual functions of ZmNF-YA3 in photoperiod-dependent flowering and abiotic stress responses in maize. Journal of Experimental Botany 69, 5177-5189, doi:10.1093/jxb/ery299 (2018).
52 Myers, Z. A. & Holt, B. F. NUCLEAR FACTOR-Y: still complex after all these years? Curr Opin Plant Biol 45, 96-102, doi:10.1016/j.pbi.2018.05.015 (2018).
53 Ly, L. L., Yoshida, H. & Yamaguchi, M. Nuclear transcription factor Y and its roles in cellular processes related to human disease. American journal of cancer research 3, 339-346 (2013).
54 Mach, J. CONSTANS Companion: CO Binds the NF-YB/NF-YC Dimer and Confers Sequence-Specific DNA Binding. Plant Cell 29, 1183, doi:10.1105/tpc.17.00465 (2017).
55 Xu, M. Y. et al. Stress-induced early flowering is mediated by miR169 in Arabidopsis thaliana. J Exp Bot 65, 89-101, doi:10.1093/jxb/ert353 (2014).
56 Liang, G., He, H. & Yu, D. Identification of nitrogen starvation-responsive microRNAs in Arabidopsis thaliana. PLoS One 7, e48951, doi:10.1371/journal.pone.0048951 (2012).
57 Schauser, L., Roussis, A., Stiller, J. & Stougaard, J. A plant regulator controlling development of symbiotic root nodules. Nature 402, 191-195, doi:10.1038/46058 (1999).
58 Ueda, Y. & Yanagisawa, S. Perception, transduction, and integration of nitrogen and phosphorus nutritional signals in the transcriptional regulatory network in plants. J Exp Bot 70, 3709-3717, doi:10.1093/jxb/erz148 (2019).
59 O'Malley, R. C. et al. Cistrome and Epicistrome Features Shape the Regulatory DNA Landscape. Cell 165, 1280-1292, doi:10.1016/j.cell.2016.04.038 (2016).
60 Kiba, T. et al. Repression of Nitrogen Starvation Responses by Members of the Arabidopsis GARP-Type Transcription Factor NIGT1/HRS1 Subfamily. Plant Cell 30, 925-945, doi:10.1105/tpc.17.00810 (2018).
61 Eulgem, T., Rushton, P. J., Robatzek, S. & Somssich, I. E. The WRKY superfamily of plant transcription factors. Trends Plant Sci 5, 199-206, doi:10.1016/s1360-1385(00)01600-9 (2000).
62 Bakshi, M. & Oelmüller, R. WRKY transcription factors: Jack of many trades in plants. Plant Signal Behav 9, e27700, doi:10.4161/psb.27700 (2014).
63 Alonso, J. M. et al. Genome-wide insertional mutagenesis of Arabidopsis thaliana. Science 301, 653-657, doi:10.1126/science.1086391 (2003).
64 Williams-Carrier, R. et al. Use of Illumina sequencing to identify transposon insertions underlying mutant phenotypes in high-copy Mutator lines of maize. The Plant Journal 63, 167-177, doi:10.1111/j.1365-313X.2010.04231.x (2010).
65 Bushnell, B. (2016).
66 Lamesch, P. et al. The Arabidopsis Information Resource (TAIR): improved gene annotation and new tools. Nucleic Acids Res 40, D1202-1210, doi:10.1093/nar/gkr1090 (2012).
67 Liao, Y., Smyth, G. K. & Shi, W. featureCounts: an efficient general purpose program for assigning sequence reads to genomic features. Bioinformatics 30, 923-930, doi:10.1093/bioinformatics/btt656 (2014).
68 Cheng, C. Y. et al. Araport11: a complete reannotation of the Arabidopsis thaliana reference genome. Plant J 89, 789-804, doi:10.1111/tpj.13415 (2017).
69 Risso, D., Schwartz, K., Sherlock, G. & Dudoit, S. GC-content normalization for RNA-Seq data. BMC Bioinformatics 12, 480, doi:10.1186/1471-2105-12-480 (2011).
70 Risso, D., Ngai, J., Speed, T. P. & Dudoit, S. Normalization of RNA-seq data using factor analysis of control genes or samples. Nat Biotechnol 32, 896-902, doi:10.1038/nbt.2931 (2014).
71 Waldmann, P. On the Use of the Pearson Correlation Coefficient for Model Evaluation in Genome-Wide Prediction. Front Genet 10, 899, doi:10.3389/fgene.2019.00899 (2019).
72 Cheng, C. Y. Evolutionarily informed machine learning enhances the power of predictive gene-to-phenotype relationships. Open Science Foundataion doi: 10.17605/OSF.IO/AVJPH (2021).

TABLE 1
Evolutionary conservation of gene responsiveness enhances
machine learning outcomes. Comparison of the performance
of maize (top) and Arabidopsis (bottom) XGBoost models
using the same number of features from different sources:
randomly selected expressed genes, top N-DEGs based on FDR
ranking in edgeR analysis, and the evolutionarily conserved
N-DEGs. The numbers indicate the P-value
of one-tailed Mann-Whitney U test.

| | Maize Features | | |
|---|---|---|---|
| | Random | | Cross Species |
| | expressed genes | Top N-DEGs | N-DEGs |
| | Pearson′s r | | |
| | r = 0.62 | r = 0.68 | r = 0.79 |
| Random | | 6.56e−04 | 1.5e−05 |
| expressed genes | | | |
| Top N-DEGs | 6.56e−04 | | 1.06E−03 |
| Cross Species | 1.5e−05 | 1.06−03 | |
| N-DEGs | | | |
| | Arabidopsis Features | | |
| | Random | | Cross Species |
| | expressed genes | Top N-DEGs | N-DEGs |
| | Pearson′s r | | |
| | r = 0.53 | r = 0.59 | r = 0.65 |
| Random | | 7.63E−06 | 3.82E−06 |
| expressed genes | | | |
| Top N-DEGs | 7.63E−06 | | 1.64E−04 |
| Cross Species | 3.82E−06 | 1.64E−04 | |
| N-DEGs | | | |

TABLE 2
Candidate TFs identified from XGBoost feature importance ranking for predicting NUE and/or
hubs in GENIE3 network constructed from XGBoost important gene features. Our validation
results confirming the roles of these eight TFs in NUE are provided in FIG. 6, and FIG. 15.

| Gene ID | Symbol | Published Functions | Selection Criteria |
|---|---|---|---|
| AT3G14020 | NF-YA6 | male gametogenesis, | At XGBoost gene-to- |
| | | embryogenesis, seed morphology, | trait model |
| | | and seed germination; ABA | |
| | | response⁴², NF-YAs are predicted | |
| | | target of miR169⁴⁵ | |
| AT4G02590 | UNE12 | temperature-responsive SA | At and Zm XGBoost |
| | | immunity regulator⁴⁷ | gene-to-trait model |
| AT5G58900 | DIV1 | Nitrogen-response gene in the | At and Zm XGBoost |
| | | Arabidopsis seedling root and | gene-to-trait model |
| | | shoot⁴⁰ | |
| AT4G18390 | TCP2 | MicroRNA-mediated leaf | At XGBoost gene-to- |
| | | morphogenesis⁴⁶, | trait model |
| | | photomorphogenesis in | |
| | | Arabidopsis⁵⁰ | |
| AT5G22570 | WRKY38 | Basal defense⁴⁸ | At GENIE3 GRN |
| AT5G26170 | WRKY50 | Systemic Acquired Resistance⁴⁹ | At GENIE3 GRN |
| AT5G06100 | MYB33 | The Arabidopsis (MYB33), maize | Zm GENIE3 GRN, At and |
| | | (Zm00001d012544) and rice | Zm XGBoost gene-to- |
| | | (OsGAMYB) homologs are | trait model, conserved |
| | | predicted target of miR159⁴⁴, | cross-species function |
| | | juvenile-to-adult transition⁴⁴, | in anther development |
| | | anther development⁴³ | |
| AT1G76350 | NLP5 | The maize homolog of NLP5 | Zm GENIE3 GRN, At and |
| | | (Zm00001d006293) is a marker for | Zm XGBoost gene-to- |
| | | N status¹⁹ and nutrient uptake⁴¹ | trait model |
| Zm00001d006835 | nfya3 | photoperiod-dependent flowering | At XGBoost gene-to- |
| | | and abiotic stress responses⁵¹ | trait model |

TABLE 3
25 MAIZE TRANSCRIPTION FACTORS AND THEIR ARABIDOPSIS HOMOLOGS

| | | Machine | | | | | Machine | | | |
|---|---|---|---|---|---|---|---|---|---|---|
| | | learning Gene | | | | | learning Gene | | | |
| | | Importance to | | | | | Importance to | | | |
| | | NUE (Cheng & | | | | | NUE (Cheng & | | | |
| | Maize | Coruzzi 2021, | | | Published | Arabidopsis | Coruzzi 2021, | | | Published |
| Row | Gene | Table S3) | Symbol | Description | Function | Gene | Table S3) | Symbol | Description | Function |
| 1 | Zm00001 | 2.1 | nf-ya3 | CCAAT-HAP2- | NA | AT3G14020 | 38.0 | NF-YA6 | nuclear factor | Table 3 |
| | d006835 | | | transcription | | | | | Y, subunit A6 | Row 1 |
| | | | | factor | | | | | | |
| 2 | Zm00001 | 41.2 | hb75 | Homeobox- | NA | AT4G04890 | 1.3 | PDF2 | protodermal | Table 3 |
| | d002234 | | | factor 75 | | | | | factor 2 | Row 46 |
| | | | | transcription | | AT4G21750 | 3.4 | ATM Li | Homeobox- | Table 3 |
| | | | | | | | | | leucine zipper | Row 22 |
| | | | | | | | | | family protein/ | |
| | | | | | | | | | lipid-binding | |
| | | | | | | | | | START domain- | |
| | | | | | | | | | containing | |
| | | | | | | | | | protein | |
| 3 | Zm00001 | 11.7 | nlp17 | NLP- | Table 2 | AT1G20640 | 18.4 | NLP4 | Plant regulator | NA |
| | d006293 | | | transcription | Row 3 | | | | RWP-RK family | |
| | | | | factor 17 | | | | | protein | |
| | | | | | | AT1G76350 | 10.7 | NLP5 | Plant regulator | NA |
| | | | | | | | | | RWP-RK family | |
| | | | | | | | | | protein | |
| 4 | Zm00001 | 7.2 | gras37 | GRAS- | NA | AT3G54220 | 7.4 | SCR | SGR1, SHOOT | Table 3 |
| | d005029 | | | transcription | | | | | GRAVITROPISM | Row 11 |
| | | | | factor 37 | | | | | 1 | |
| 5 | Zm00001 | 6.4 | sbp23 | SBP- | NA | AT3G57920 | 0.6 | SPL15 | squamosa | Table 3 |
| | d006028 | | | transcription | | | | | promoter | Row 54 |
| | | | | factor 23 | | | | | binding | |
| | | | | | | | | | protein-like 15 | |
| 6 | Zm00001 | 10.2 | hb66 | Homeobox- | NA | AT3G61890 | 0.6 | HB-12 | ATHB-12, | Table 3 |
| | d002799 | | | factor 66 | | | | | homeobox 12 | Row 55 |
| | | | | transcription | | AT2G46680 | 0.6 | HB-7 | ATHB-7, | Table 3 |
| | | | | | | | | | homeobox 7 | Row 53 |
| 7 | Zm00001 | 2.3 | abi28 | ABI3-VP1- | NA | AT2G24645 | 1.6 | | Transcriptional | NA |
| | d004358 | | | transcription | | | | | factor B3 | |
| | | | | factor 28 | | | | | family protein | |
| 8 | Zm00001 | 2.1 | bbx6 | b-box6 | Table 2 | AT2G21320 | 0.3 | BBX18 | B-box zinc | Table 3 |
| | d006198 | | | | Row 8 | | | | finger family | Row 62 |
| | | | | | | | | | protein | |
| 9 | Zm00001 | 4.2 | arr8 | ARR-B- | NA | AT2G25180 | 2.3 | RR12 | ARR12, | Table 3 |
| | d018380 | | | transcription | | | | | response | Row 30 |
| | | | | factor 8 | | | | | regulator 12 | |
| 10 | Zm00001 | 4.0 | nf-ya11 | CCAAT-HAP2- | NA | AT3G05690 | 4.0 | NF-YA2 | nuclear factor | Table 3 |
| | d013676 | | | factor 210 | | | | | Y, subunit A2 | Row 19 |
| | | | | transcription | | AT5G06510 | 2.5 | NF-YA10 | nuclear factor | Table 3 |
| | | | | | | | | | Y, subunit A10 | Row 27 |
| 11 | Zm00001 | 0.8 | bhlh15 | bHLH- | NA | AT1G03040 | 14.5 | | basic helix- | Table 3 |
| | d013073 | | 9 | transcription | | | | | loop-helix | Row 6 |
| | | | | factor 159 | | | | | (bHLH) DNA- | |
| | | | | | | | | | binding | |
| | | | | | | | | | superfamily | |
| | | | | | | | | | protein | |
| | | | | | | AT4G02590 | 12.8 | UNE12 | basic helix- | Table 3 |
| | | | | | | | | | loop-helix | Row 7 |
| | | | | | | | | | (bHLH) DNA- | |
| | | | | | | | | | binding | |
| | | | | | | | | | superfamily | |
| | | | | | | | | | protein | |
| 12 | Zm00001 | 0.1 | myb38 | myb | Table 2 | AT4G38620 | 1.8 | MYB4 | ATMYB4, myb | Table 3 |
| | d032024 | | | transcription | Row 12 | | | | domain protein | Row 37 |
| | | | | factor38 | | | | | 4 | |
| 13 | Zm00001 | 0.6 | nlp13 | NLP- | Table 2 | AT1G20640 | 18.4 | NLP4 | Plant regulator | NA |
| | d021442 | | | transcription | Row 13 | | | | RWP-RK family | |
| | | | | factor 13 | | | | | protein | |
| | | | | | | AT1G76350 | 10.7 | NLP5 | Plant regulator | NA |
| | | | | | | | | | RWP-RK family | |
| | | | | | | | | | protein | |
| 14 | Zm00001 | 0.3 | myb74 | MYB- | Table 2 | AT5G06100 | 1.3 | MYB33 | | Table 3 |
| | d012544 | | | transcription | Row 14 | | | | | Row 45 |
| | | | | factor 74 | | | | | | |
| 15 | Zm00001 | 0.5 | c3h39 | C3H- | NA | AT2G19810 | 4.6 | OZF1 | AtOZF1, AtTZF2, | Table 3 |
| | d037769 | | | transcription | | | | | TZF2, tandem | Row 15 |
| | | | | factor 39 | | | | | zinc finger 2 | |
| 16 | Zm00001 | 0.3 | myb34 | MYB- | NA | AT5G58900 | 15.2 | DIV1 | Homeodomain- | Table 3 |
| | d042830 | | | transcription | | | | | like | Row 5 |
| | | | | factor 34 | | | | | transcriptional | |
| | | | | | | | | | regulator | |
| 17 | Zm00001 | 0.9 | ereb81 | AP2-EREBP- | Table 2 | AT2G28550 | 1.8 | RAP2.7 | TO E1, TARGET | Table 3 |
| | d035512 | | | transcription | Row 17 | | | | OF EARLY | Row 35 |
| | | | | factor 81 | | | | | ACTIVATION | |
| | | | | | | | | | TAGGED (EAT) | |
| | | | | | | | | | 1 | |
| 18 | Zm00001 | 1.1 | nactf10 | NAC- | Table 2 | AT1G01720 | 0.9 | ATAF1 | NAC (No Apical | Table 3 |
| | d042609 | | 9 | transcription | Row 18 | | | | Meristem) | Row 50 |
| | | | | factor 109 | | | | | domain | |
| | | | | | | | | | transcriptional | |
| | | | | | | | | | regulator | |
| | | | | | | | | | superfamily | |
| | | | | | | | | | protein | |
| 19 | Zm00001 | 0.1 | wrky40 | WRKY- | Table 2 | AT5G22570 | 1.8 | WRKY38 | ATWRKY38, AR | Table 3 |
| | d043062 | | | transcription | Row 19 | | | | ABIDOPSIS | Row 38 |
| | | | | factor 40 | | | | | THALIANA | |
| | | | | | | | | | WRKY DNA- | |
| | | | | | | | | | BINDING | |
| | | | | | | | | | PROTEIN 38 | |
| 20 | Zm00001 | 0.3 | mybr3 | MYB-related- | | AT5G58900 | 15.2 | DIV1 | Homeodomain- | Table 3 |
| | d038270 | | | transcription | | | | | like | Row 5 |
| | | | | factor 3 | | | | | transcriptional | |
| | | | | | | | | | regulator | |
| 21 | Zm00001 | 0.1 | bzip10 | bZIP- | Table 2 | AT1G77920 | 2.4 | TGA7 | bZIP | Table 3 |
| | d024160 | | 7 | transcription | Row 21 | | | | transcription | Row 29 |
| | | | | factor 107 | | | | | factor family | |
| | | | | | | | | | protein | |
| 22 | Zm00001 | 0.3 | wrky58 | WRKY- | NA | AT1G13960 | 1.5 | WRKY4 | WRKY DNA- | Table 3 |
| | d041740 | | | transcription | | | | | binding protein | Row 42 |
| | | | | factor 58 | | | | | 4 | |
| 23 | Zm00001 | 0.1 | nlp6 | NLP- | NA | AT5G24310 | 4.9 | ABIL3 | ABL interactor- | |
| | d039266 | | | transcription | | | | | like protein 3 | |
| | | | | factor 6 | | | | | | |
| 24 | Zm00001 | 0.1 | nactf44 | NAC- | NA | AT3G04070 | 1.2 | NAC047 | ANAC047, NAC | Table 3 |
| | d028999 | | | transcription | | | | | domain | Row 47 |
| | | | | factor 44 | | | | | containing | |
| | | | | | | | | | protein 47, | |
| | | | | | | | | | SHG, SPEEDY | |
| | | | | | | | | | HYPONASTIC | |
| | | | | | | | | | GROWTH | |
| 25 | Zm00001 | 0.1 | wrky12 | WRKY- | NA | AT5G26170 | 0.2 | WRKY50 | ATWRKY50, | Table |
| | d037607 | | 5 | transcription | | | | | ARABIDOPSIS | Row 63 |
| | | | | factor 125 | | | | | THALIANA | |
| | | | | | | | | | WRKY DNA- | |
| | | | | | | | | | BINDING | |
| | | | | | | | | | PROTEIN 50 | |

TABLE 4
25 MAIZE TRANSCRIPTION FACTORS

| | | | | Machine | | | | | | | |
|---|---|---|---|---|---|---|---|---|---|---|---|
| | | | | learning | | | | | Validation | | |
| | | | | Gene | | | Ranking: | Ranking: | of role in | | |
| | | | | Importance | | | # of | # of | NUE using | | |
| | | | | to NUE | | Ranking: # | target as | target as | mutant | | |
| | | | | (Cheng & | | of target in | gene | diff- | (Cheng & | | |
| | | | | Coruzzi | | N- | features | erentially | Coruzzi | Published | Published |
| | | | | 2021, Table | Rank- | assimilation | predictive | expressed | 2021, FIG. | N | non-N |
| Row | Gene | Symbol | Description | S3) | sum | pathways | of NUE | TF | 6) | function | function |
| 1 | Zm00001 | nf-ya3 | CCAAT-HAP2- | 2.1 | 46 | 7 | 23 | 16 | Yes | NA | Photoperiod- |
| | d006835 | | transcription | | | | | | | | dependent |
| | | | factor4 | | | | | | | | flowering |
| | | | | | | | | | | | time control |
| | | | | | | | | | | | (Su et al., |
| | | | | | | | | | | | 2018) |
| 2 | Zm00001 | hb75 | Homeobox- | 41.2 | 41 | 19 | 14 | 8 | | NA | NA |
| | d002234 | | transcription | | | | | | | | |
| | | | factor 75 | | | | | | | | |
| 3 | Zm00001 | nlp27 | NLP- | 11.7 | 17 | 7 | 3 | 7 | | N status | Ion Uptake in |
| | d006293 | | transcription | | | | | | | biomarker | the root |
| | | | factor 17 | | | | | | | (Yang et | (Griffiths et |
| | | | | | | | | | | al., 2011) | al., 2020) |
| 4 | Zm00001 | gras37 | GRAS- | 7.2 | 54 | 12 | 22 | 20 | | NA | NA |
| | d005029 | | transcription | | | | | | | | |
| | | | factor 37 | | | | | | | | |
| 5 | Zm00001 | sbp23 | SBP- | 6.4 | 71 | 22 | 24 | 25 | | NA | NA |
| | d006028 | | transcription | | | | | | | | |
| | | | factor 23 | | | | | | | | |
| 6 | Zm00001 | hb66 | Homeobox- | 10.2 | 25 | 7 | 15 | 3 | | NA | NA |
| | d002799 | | transcription | | | | | | | | |
| | | | factor 66 | | | | | | | | |
| 7 | Zm00001 | abi28 | ABI3-VP1- | 2.3 | 27 | 3 | 13 | 11 | | NA | NA |
| | d004358 | | transcription | | | | | | | | |
| | | | factor 28 | | | | | | | | |
| 8 | Zm00001 | bbx6 | b-box6 | 2.1 | 26 | 14 | 6 | 6 | | NA | Leaf |
| | d006198 | | | | | | | | | | senescence |
| | | | | | | | | | | | (Sekhon et |
| | | | | | | | | | | | al., 2019) |
| 9 | Zm00001 | arr8 | ARR-B- | 4.2 | 8 | 2 | 5 | 1 | | NA | NA |
| | d018380 | | transcription | | | | | | | | |
| | | | factor 8 | | | | | | | | |
| 10 | Zm00001 | nf-ya11 | CCAAT-HAP2- | 4.0 | 39 | 14 | 11 | 14 | | NA | NA |
| | d013676 | | transcription | | | | | | | | |
| | | | factor 210 | | | | | | | | |
| 11 | Zm00001 | bhlh159 | bHLH- | 0.8 | 63 | 23 | 19 | 21 | | NA | NA |
| | d013073 | | transcription | | | | | | | | |
| | | | factor 159 | | | | | | | | |
| 12 | Zm00001 | myb38 | myb | 0.1 | 68 | 24 | 21 | 23 | | Misregulated | NA |
| | d032024 | | transcription | | | | | | | in | |
| | | | factor38 | | | | | | | mop1 | |
| | | | | | | | | | | mutant | |
| | | | | | | | | | | (Vendra | |
| | | | | | | | | | | min et | |
| | | | | | | | | | | al., 2020) | |
| 13 | Zm00001 | nlp13 | NLP- | 0.6 | 29 | 7 | 8 | 14 | | Drought- | NA |
| | d021442 | | transcription | | | | | | | responsive | |
| | | | factor 13 | | | | | | | (Jin et | |
| | | | | | | | | | | al., 2019) | |
| 14 | Zm00001 | myb74 | MYB- | 0.3 | 39 | 6 | 16 | 17 | | Potential | NA |
| | d012544 | | transcription | | | | | | | targets of | |
| | | | factor 74 | | | | | | | microRNA | |
| | | | | | | | | | | (Li et | |
| | | | | | | | | | | al., 2019) | |
| 15 | Zm00001 | c3h39 | C3H- | 0.5 | 13 | 7 | 2 | 4 | | NA | NA |
| | d037769 | | transcription | | | | | | | | |
| | | | factor 39 | | | | | | | | |
| 16 | Zm00001 | myb34 | MYB- | 0.3 | 19 | 14 | 4 | 1 | | NA | NA |
| | d042830 | | transcription | | | | | | | | |
| | | | factor 34 | | | | | | | | |
| 17 | Zm00001 | ereb81 | AP2-EREBP- | 0.9 | 74 | 25 | 25 | 24 | | Stress- | NA |
| | d035512 | | transcription | | | | | | | responsive | |
| | | | factor 81 | | | | | | | (Du et | |
| | | | | | | | | | | al., 2014) | |
| 18 | Zm00001 | nactf10 | NAC- | 1.1 | 33 | 14 | 7 | 12 | | Overexpression | NA |
| | d042609 | 9 | transcription | | | | | | | in | |
| | | | factor 109 | | | | | | | Arabidopsis | |
| | | | | | | | | | | enhance | |
| | | | | | | | | | | drought | |
| | | | | | | | | | | tolerance | |
| | | | | | | | | | | (Liu et | |
| | | | | | | | | | | al., 2019) | |
| 19 | Zm00001 | wrky40 | WRKY- | 0.1 | 34 | 3 | 18 | 13 | | Misregulated | NA |
| | d043062 | | transcription | | | | | | | in | |
| | | | factor 40 | | | | | | | mop1 | |
| | | | | | | | | | | mutant | |
| | | | | | | | | | | (Vendramin | |
| | | | | | | | | | | et | |
| | | | | | | | | | | al., 2020) | |
| 20 | Zm00001 | mybr3 | MYB-related- | 0.3 | 13 | 3 | 1 | 9 | | NA | NA |
| | d038270 | | transcription | | | | | | | | |
| | | | factor 3 | | | | | | | | |
| 21 | Zm00001 | bzip107 | bZIP- | 0.1 | 33 | 12 | 12 | 9 | | Misregulated | NA |
| | d024160 | | transcription | | | | | | | in | |
| | | | factor 107 | | | | | | | mop1 | |
| | | | | | | | | | | mutant | |
| | | | | | | | | | | (Vendramin | |
| | | | | | | | | | | et | |
| | | | | | | | | | | al., 2020) | |
| 22 | Zm00001 | wrky58 | WRKY- | 0.3 | 61 | 19 | 20 | 22 | | NA | NA |
| | d041740 | | transcription | | | | | | | | |
| | | | factor 58 | | | | | | | | |
| 23 | Zm00001 | nlp6 | NLP- | 0.1 | 54 | 19 | 17 | 18 | | NA | NA |
| | d039266 | | transcription | | | | | | | | |
| | | | factor 6 | | | | | | | | |
| 24 | Zm00001 | nactf44 | NAC- | 0.1 | 15 | 1 | 9 | 5 | | NA | NA |
| | d028999 | | transcription | | | | | | | | |
| | | | factor 44 | | | | | | | | |
| 25 | Zm00001 | wrky125 | WRKY- | 0.1 | 47 | 18 | 10 | 19 | | NA | NA |
| | d037607 | | transcription | | | | | | | | |
| | | | factor 125 | | | | | | | | |

TABLE 5
63 ARABIDOPSIS TRANSCRIPTION FACTORS

| | | | | Machine | Validation | | |
|---|---|---|---|---|---|---|---|
| | | | | learning | of role in | | |
| | | | | Gene | NUE using | Published | |
| | | | | Importance | mutant | Nitrogen | |
| | | | | to NUE | (Cheng & | function | Published |
| | | | | (Cheng & | Coruzzi | using | non-Nitrogen function |
| | | | | Coruzzi 2021, | 2021, | mutants/ | using |
| Row | Gene | Symbol | Description | Table S3) | FIG. 6) | transgenics | mutants/transgenics |
| 1 | AT3G14020 | NF-YA6 | nuclear factor Y, subunit A6 | 38.0 | Yes | NA | Male gametogenesis, |
| | | | | | | | embryogenesis, and |
| | | | | | | | seed development |
| | | | | | | | (Mu et al., 2013) |
| 2 | AT1G54160 | NF-YA5 | NFYA5, NUCLEAR FACTOR Y A5 | 22.8 | | NA | Drought resistance (Li |
| | | | | | | | et al., 2008) |
| 3 | AT1G20640 | NLP4 | Plant regulator RWP-RK family protein | 18.4 | | NA | NA |
| 4 | AT3G09370 | MYB3R-3 | AtMYB3R3, myb domain protein 3R3 | 16.8 | | NA | DNA repair |
| | | | | | | | (Bourbousse et al., |
| | | | | | | | 2018) |
| 5 | AT5G58900 | DIV1 | Homeodomain-like transcriptional | 15.2 | Yes | NA | NA |
| | | | regulator | | | | |
| 6 | AT1G03040 | | basic helix-loop-helix (bHLH) DNA- | 14.5 | | NA | Thermoresponsive |
| | | | binding superfamily protein | | | | regulator (Bruessow et |
| | | | | | | | al., 2021) |
| 7 | AT4G02590 | UNE12 | basic helix-loop-helix (bHLH) DNA- | 12.8 | Yes | NA | Thermoresponsive |
| | | | binding superfamily protein | | | | regulator (Bruessow et |
| | | | | | | | al., 2021) |
| 8 | AT1G76350 | NLP5 | Plant regulator RWP-RK family protein | 10.7 | Yes | NA | NA |
| 9 | AT5G08190 | NF-YB12 | nuclear factor Y, subunit B12 | 9.4 | | NA | NA |
| 10 | AT5G20510 | AL5 | alfin-like 5 | 8.1 | | NA | Abiotic stress |
| | | | | | | | tolerance (Wei ei al., |
| | | | | | | | 2015) |
| 11 | AT3G54220 | SCR | SGR1, SHOOT GRAVITROPISM 1 | 7.4 | | NA | Root development (Di |
| | | | | | | | Laurenzio et al., 1996), |
| | | | | | | | Bundle sheath |
| | | | | | | | differentiation (Cui et |
| | | | | | | | al., 2014) |
| 12 | AT4G39250 | RL1 | ATRL1, RAD-like 1, RSM2, | 6.9 | | NA | NA |
| | | | RADIALIS-LIKE SANT/MYB 2 | | | | |
| 13 | AT3G46130 | MYB48 | ATMYB48-1, ATMYB48-2, | 6.8 | | NA | NA |
| | | | ATMYB48-3, ATMYB48, | | | | |
| | | | myb domain protein 48 | | | | |
| 14 | AT4G26150 | CGA1 | GATA22, GATA TRANSCRIPTION | 4.7 | | Nitrate- | Flowering time and |
| | | | FACTOR 22, GNL, GNC-LIKE | | | responsive | cold tolerance (Ritcher |
| | | | | | | and | et al., 2013) |
| | | | | | | chlorophyll | |
| | | | | | | synthesis | |
| | | | | | | (Bi et all, | |
| | | | | | | 2005) | |
| 15 | AT2G19810 | OZF1 | AtOZF1, AtTZF2, TZF2, tandem zinc | 4.6 | | NA | JA and ABA response |
| | | | finger 2 | | | | (Lee et al., 2012) |
| 16 | AT4G35270 | NLP2 | Plant regulator RWP-RK family protein | 4.5 | | NA | NA |
| 17 | AT5G6541O | HB25 | ATHB25, ARABIDOPSIS THALIANA | 4.4 | | NA | Gibberellin signalling |
| | | | HOMEOBOX PROTEIN | | | | in seed longevity |
| | | | 25, ZFHD2, ZINC | | | | (Bueso et al., 2012) |
| | | | FINGER HOMEODOMAIN | | | | |
| | | | 2, ZHD1, ZINC | | | | |
| | | | FINGER HOMEODOMAIN 1 | | | | |
| 18 | AT1G78600 | LZF1 | BBX22, B-box domain protein | 4.0 | | NA | Photomorphogenesis |
| | | | 22, DBB3, DOUBLE | | | | (Gangappa et al., |
| | | | B-BOX 3, STH3,SALT | | | | 2013) |
| | | | TOLERANCE HOMOLOG 3 | | | | NA |
| 19 | AT3G05690 | NF-YA2 | ATHAP2B, HEME | 4.0 | | NA | |
| | | | ACTIVATOR PROTEIN | | | | |
| | | | (YEAST) HOMOLOG 2B, AtNF- | | | | |
| | | | YA2, HAP2B, HEME ACTIVATOR | | | | |
| | | | PROTEIN (YEAST) HOMOLOG | | | | |
| | | | 2B, UNE8, UNFERTILIZED | | | | |
| | | | EMBRYO SAC 8 | | | | |
| 20 | AT1G30500 | NF-YA7 | nuclear factor Y, subunit A7 | 3.8 | | NA | Abiotic stress |
| | | | | | | | tolerance (Leyva- |
| | | | | | | | González et al., 2012) |
| 21 | AT3G20640 | | basic helix-loop-helix (bHLH) DNA- | 3.6 | | NA | Cell elongation and |
| | | | binding superfamily protein | | | | seed germination (Lee |
| | | | | | | | et al., 2005) |
| 22 | AT4G21750 | ATML1 | Homeobox-leucine zipper family | 3.4 | | NA | Shoot epidermal cell |
| | | | protein/lipid-binding START domain- | | | | differentiation (Takada |
| | | | containing protein | | | | et al., 2013) |
| 23 | AT3G59580 | NLP9 | Plant regulator RWP-RK family protein | 3.3 | | NA | NA |
| 24 | AT2G34720 | NF-YA4 | nuclear factor Y, subunit A4 | 3.0 | | NA | NA |
| 25 | AT2G43500 | NLP8 | Plant regulator RWP-RK | 3.0 | | Nitrate- | NA |
| | | | family protein | | | promoted | |
| | | | | | | seed | |
| | | | | | | germination | |
| | | | | | | (Yan et al., | |
| | | | | | | 2016) | |
| 26 | AT3G15270 | SPL5 | squamosa promoter binding protein- | 2.7 | | Nitrate- | Flowering time (Lal et |
| | | | like 5 | | | mediated | al., 2011) |
| | | | | | | flowering time | |
| | | | | | | control | |
| | | | | | | (Olas et al., | |
| | | | | | | 2019) | |
| 27 | AT5G06510 | NF-YA10 | nuclear factor Y, subunit A10 | 2.5 | | NA | Leaf growth via auxin |
| | | | | | | | signaling (Zhang et al., |
| | | | | | | | 2017) |
| 28 | AT5G24930 | COL4 | ATCOL4, BBX5, B-box | 2.4 | | NA | Abiotic stress |
| | | | domain protein 5 | | | | tolerance (Min et al., |
| | | | | | | | 2015) |
| 29 | AT1G77920 | TGA7 | bZIP transcription factor family | 2.4 | | NA | Disease resistance |
| | | | protein | | | | (Kesarwani et al., |
| | | | | | | | 2007) |
| 30 | AT2G25180 | RR12 | ARR12, response regulator | 2.3 | | NA | Cytokinin signal |
| | | | 12, AtARR12 | | | | transduction (Mason |
| | | | | | | | et al., 2005) |
| 31 | AT3G20910 | NF-YA9 | nuclear factor Y, subunit A9 | 2.2 | | NA | Male gametogenesis, |
| | | | | | | | embryogenesis, and |
| | | | | | | | seed development |
| | | | | | | | (Mu et al., 2013) |
| 32 | AT4G18390 | TCP2 | TEOSINTE BRANCHED 1, cycloidea | 2.2 | Yes | NA | Photomorphogenesis |
| | | | and PCF transcription factor 2 | | | | (He et al., 2016) |
| 33 | AT1G19510 | RL5 | ATRL5, RAD-like 5, RSM4, | 2.0 | | NA | NA |
| | | | RADIALIS-LIKE SANT/MYB 4 | | | | |
| 34 | AT1G72650 | TRFL6 | TRF-like 6 | 1.9 | | NA | NA |
| 35 | AT2G28550 | RAP2.7 | TOE1, TARGET OF | 1.8 | | NA | Flowering time and |
| | | | EARLY ACTIVATION | | | | innate immunity (Zhai |
| | | | TAGGED (EAT) 1 | | | | et al., 2015) |
| 36 | AT2G42280 | FBH4 | AKS3, ABA-responsive kinase | 1.8 | | NA | Flowering time (Ito et |
| | | | substrate 3 | | | | al., 2012) |
| 37 | AT4G38620 | MYB4 | ATMYB4, myb domain protein 4 | 1.8 | | NA | Flavonoid biosynthesis |
| | | | | | | | (Wang et al., 2020) |
| 38 | AT5G22570 | WRKY38 | ATWRKY38, | 1.8 | Yes | NA | Plant defense (Kim et |
| | | | ARABIDOPSIS THALIANA | | | | al., 2008) |
| | | | WRKY DNA-BINDING PROTEIN 38 | | | | |
| 39 | AT5G59780 | MYB59 | ATMYB59-1, ATMYB59-2, | 1.7 | | NA | NA |
| | | | ATMYB59-3, ATMYB59, MYB | | | | |
| | | | DOMAIN PROTEIN 59 | | | | |
| 40 | AT1G30210 | TCP24 | ATTCP24 | 1.7 | | NA | Secondary cell wall |
| | | | | | | | thickening and anther |
| | | | | | | | endothecium (Wang et |
| | | | | | | | al., 2015) |
| 41 | AT2G24645 | | Transcriptional factor B3 family | 1.6 | | NA | NA |
| | | | protein | | | | |
| 42 | AT1G13960 | WRKY4 | WRKY DNA-binding protein 4 | 1.5 | | NA | Plant resistance to |
| | | | | | | | biotrophic pathogens |
| | | | | | | | (Lai et al., 2008) |
| 43 | AT5G67420 | LBD37 | ASL39, ASYMMETRIC | 1.4 | | Anthocyanin | NA |
| | | | LEAVES2-LIKE 39 | | | synthesis and | |
| | | | | | | nitrogen | |
| | | | | | | responses | |
| | | | | | | (Rubin et | |
| | | | | | | al., 200) | |
| 44 | AT2G16770 | bZIP23 | Basic-leucine zipper (bZIP) | 1.4 | | NA | Zinc sensor (Lilav et al., |
| | | | transcription factor family protein | | | | 2021) |
| 45 | AT5G06100 | MYB33 | ATMYB33 | 1.3 | Yes | NA | Regulated by miR159 |
| | | | | | | | in anther development |
| | | | | | | | (Miller and Gubler, |
| | | | | | | | 2005) |
| 46 | AT4G04890 | PDF2 | protodermal factor 2 | 1.3 | | NA | Embryo development |
| | | | | | | | (Ogawa et al., 2015) |
| 47 | AT3G04070 | NAC047 | ANAC047, NAC domain containing | 1.2 | | NA | Waterlogging-induced |
| | | | protein 47, SHG, SPEEDY | | | | hyponastic leaf growth |
| | | | HYPONASTIC GROWTH | | | | (Rauf et al., 2013) |
| 48 | AT3G42790 | AL3 | alfin-like 3 | 0.9 | | NA | NA |
| 49 | AT2G02470 | AL6 | alfin-like 6 | 0.9 | | NA | Abiotic stress (Wei et |
| | | | | | | | al., 2015) |
| 50 | AT1G01720 | ATAF1 | NAC (No Apical Meristem) domain | 0.9 | | NA | Embryogenesis |
| | | | transcriptional regulator superfamily | | | | (Kunieda et al., 2008) |
| | | | protein | | | | |
| 51 | AT3G19510 | HAT3.1 | Homeodomain-like protein with | 0.8 | | NA | NA |
| | | | RING/FYVE/PHD-type zinc finger | | | | |
| | | | domain-containing protein | | | | |
| 52 | AT1G56170 | NF-YC2 | ATHAP5B, HAP5B | 0.7 | | NA | Flowering (Hackenberg |
| | | | | | | | et al., 2012) |
| 53 | AT2G46680 | HB-7 | ATHB-7, homeobox | 0.6 | | NA | Drought response (Re |
| | | | 7, ATHB7, ARABIDOPSIS THALIANA | | | | et al., 2014) |
| | | | HOMEOBOX7 | | | | |
| 54 | AT3G57920 | SPL15 | squamosa promoter binding protein- | 0.6 | | NA | Flowering (Hyun et al., |
| | | | like 15 | | | | 2016) |
| 55 | AT3G61890 | HB-12 | ATHB-12, homeobox | 0.6 | | NA | Drought response (Re |
| | | | 12, ATHB12, ARABIDOPSIS THALIANA | | | | et al., 2014) |
| | | | HOMEOBOX 12 | | | | |
| 56 | AT1G53160 | SPL4 | FTM6, FLORAL TRANSITION | 0.5 | | NA | Flowering (Jung et al., |
| | | | AT THE MERISTEM6 | | | | 2016) |
| 57 | AT5G11510 | MYB3R-4 | AtMYB3R4, myb domain protein 3R4 | 0.5 | | NA | Cell cycle (Haga et al., |
| | | | | | | | 2011) |
| 58 | AT1G14920 | GAI | RGA2, RESTORATION ON | 0.5 | | NA | Gibberellin |
| | | | GROWTH ON AMMONIA 2 | | | | responses (Peng et al., |
| | | | | | | | 1997) |
| 59 | AT2G21230 | bZIP30 | Basic-leucine zipper (bZIP) | 0.4 | | NA | Reproductive |
| | | | transcription factor family protein | | | | development (Lozano- |
| | | | | | | | Sotomayor et al., |
| | | | | | | | 2016) |
| 60 | AT2G27230 | LHW | transcription factor-like protein | 0.4 | | NA | Epidermal responses |
| | | | | | | | to phosophate |
| | | | | | | | deprivation (Wendrich |
| | | | | | | | et al., 2020) |
| 61 | AT3G49940 | LBD38 | LOB domain-containing protein 38 | 0.4 | | Anthocyanin | |
| | | | | | | synthesis and | |
| | | | | | | nitrogen | |
| | | | | | | responses | |
| | | | | | | (Rubin et | |
| | | | | | | al., 200) | |
| 62 | AT2G21320 | BBX18 | B-box zinc finger family protein | 0.3 | | NA | Thermomorphogenesis |
| | | | | | | | (Ding et al., 2018) |
| 63 | AT5G26170 | WRKY50 | ATWRKY50, | 0.2 | Yes | NA | Plant defense |
| | | | ARABIDOPSIS THALIANA | | | | (Hussain |
| | | | WRKY DNA-BINDING PROTEIN 50 | | | | et al., 2018) |

TABLE 6
209 MAIZE NON-TRANSCRIPTION FACTORS AND THEIR ARABIDOPSIS HOMOLOGS

| | | Machine learning | | | | Machine learning | | |
|---|---|---|---|---|---|---|---|---|
| | | Gene Im- portance | | | | Gene Im- portance | | |
| | | to NUE | | | | to NUE | | |
| | | (Cheng & Coruzzi | | | | (Cheng & Coruzzi | | |
| Row | Maize Gene | 2021, Table S3) | Symbol | Description | Arabidopsis Gene | 2021, Table S3) | Symbol | Description |
| 1 | Zm00001d0 | 128.9 | morf2 | multiple | AT4G09010 | 0.2 | TL29 | APX4, ascorbate peroxidase 4 |
| | 02426 | | | organellar RNA | | | | |
| 2 | Zm00001d0 | 96.5 | | editing factor2 | AT1G15820 | 3.1 | LHCB6 | CP24 |
| | 01857 | | | | | | | |
| 3 | Zm00001d0 | 90.8 | | | AT3G15360 | 1.2 | TRX-M4 | ATHM4, ATM4, ARABIDOPSIS |
| | 02854 | | | | | | | THIOREDOXIN M-TYPE 4 |
| 4 | Zm00001d0 | 74.4 | mlo9 | barley mlo defense | AT5G53760 | 2.6 | MLO11 | ATM LO11, MILDEW |
| | 01804 | | | gene homolog9 | | | | RESISTANCE LOCUS O 11 |
| 5 | Zm00001d0 | 71.4 | imd2 | isopropylmalate | AT5G14200 | 0.5 | IMD1 | ATIMD1, ARABIDOPSIS |
| | 02880 | | | dehydrogenase2 | | | | ISOPROPYLMALATE |
| | | | | | | | | DEHYDROGENASE 1 |
| 6 | Zm00001d0 | 59.7 | pco139896 | Photosystem I | AT1G08380 | 1.9 | PSAO | photosystem I subunit O |
| | 03767 | | | subunit O | | | | |
| 7 | Zm00001d0 | 51.0 | | Probable | AT1G08630 | 18.0 | THA1 | threonine aldolase 1 |
| | 03059 | | | low-specificity | | | | |
| | | | | L-threonine aldolase 1 | | | | |
| 8 | Zm00001d0 | 40.8 | | Peroxisomal (S)-2- | AT4G18360 | 1.5 | GOX3 | Aldolase-type TIM barrel |
| | 02261 | | | hydroxy-acid oxidase | | | | family protein |
| | | | | GLO1 | | | | |
| 9 | Zm00001d0 | 39.3 | | OJ000126_13.10 | AT4G26950 | 2.0 | | senescence regulator |
| | 02798 | | | protein | | | | (Protein of unknown |
| | | | | | | | | function, DUF584) |
| 10 | Zm00001d0 | 38.5 | | ABC transporter | AT3G60160 | 0.3 | ABCC9 | ATMRP9, multidrug |
| | 02503 | | | C family | | | | resistance-associated protein |
| | | | | member 9 | | | | 9, MRP9, multidrug |
| | | | | | | | | resistance-associated protein |
| | | | | | | | | 9 |
| 11 | Zm00001d0 | 36.7 | | Photosystem | AT4G28750 | 0.8 | PSAE-1 | Photosystem I reaction |
| | 05446 | | | I reaction | | | | centre subunit IV/PsaE |
| | | | | center subunit IV A | | | | protein |
| | | | | | AT2G20260 | 0.4 | PSAE-2 | photosystem I subunit E-2 |
| 12 | Zm00001d0 | 30.3 | SAUR11 | auxin-responsive | AT2G45210 | 2.0 | SAUR36 | SAG201, senescence- |
| | 02826 | | | SAUR | | | | associated gene 201 |
| | | | | family member | AT3G60690 | 0.2 | SAUR59 | SMALL AUXIN |
| | | | | | | | | UPREGULATED |
| | | | | | | | | RNA 59 |
| 13 | Zm00001d0 | 26.9 | | Cyclopropane fatty | AT3G23530 | 4.3 | | Cyclopropane-fatty-acyl- |
| | 06098 | | | acid synthase | | | | phospholipid synthase |
| 14 | Zm00001d0 | 26.1 | cys1 | cysteine synthase1 | AT2G43750 | 1.4 | OASB | ACS1, ARABIDOPSIS |
| | 08379 | | | | | | | CYSTEINE |
| | | | | | | | | SYNTHASE 1, ATCS- |
| | | | | | | | | B, ARABIDOPSIS THALIANA |
| | | | | | | | | CYSTEIN SYNTHASE- |
| | | | | | | | | B, CPACS1, CHLOROPLAST |
| | | | | | | | | O-ACETYLSERINE |
| | | | | | | | | SULFHYDRYLASE 1 |
| 15 | Zm00001d0 | 20.8 | | Probable metal- | AT5G41000 | 0.7 | YSL4 | AtYSL4 |
| | 03941 | | | nicotianamine | | | | |
| | | | | transporter YSL6 | | | | |
| 16 | Zm00001d0 | 18.6 | hct5 | hydroxycinnamoyl- | AT5G48930 | 5.1 | HCT | hydroxycinnamoyl-CoA |
| | 03129 | | | transferase5 | | | | shikimate/quinate |
| | | | | | | | | hydroxycinnamoyl |
| | | | | | | | | transferase |
| 17 | Zm00001d0 | 17.5 | | PLAT domain- | AT2G22170 | 0.6 | PLAT2 | Lipase/lipooxygenase, |
| | 03457 | | | containing | | | | PLAT/LH2 family protein |
| | | | | protein 3 | | | | |
| 18 | Zm00001d0 | 17.1 | pco080190 | Amino acid binding | AT2G36840 | 2.0 | ACR10 | ACT-like superfamily protein |
| | 05317 | | | protein | | | | |
| 19 | Zm00001d0 | 16.1 | | Cysteine-rich | AT4G23180 | 2.4 | CRK10 | RLK4 |
| | 06793 | | | receptor-like | AT4G23150 | 1.3 | CRK7 | cysteine-rich RLK (RECEPTOR- |
| | | | | protein kinase 10 | | | | like protein kinase) 7 |
| | | | | | AT4G23130 | 0.3 | CRK5 | RLK6, RECEPTOR-LIKE |
| | | | | | | | | PROTEIN KINASE 6 |
| | | | | | AT4G23140 | 0.1 | CRK6 | cysteine-rich RLK (RECEPTOR- |
| | | | | | | | | like protein kinase) 6 |
| | | | | | AT4G11530 | 0.7 | CRK34 | cysteine-rich RLK (RECEPTOR- |
| | | | | | | | | like protein kinase) 34 |
| | | | | | AT4G23230 | 0.9 | CRK15 | cysteine-rich RECEPTOR-like |
| | | | | | | | | kinase |
| 20 | Zm00001d0 | 15.3 | ga2ox2 | gibberellin | AT4G21200 | 9.1 | GA20X8 | ATGA20X8, ARABIDOPSIS |
| | 02999 | | | 2-oxidase2 | | | | THALIANA GIBBERELLIN 2- |
| | | | | | | | | OXIDASE 8 |
| 21 | Zm00001d0 | 15.0 | elip1 | early light inducible | AT3G22840 | 0.2 | ELIP1 | ELIP |
| | 07827 | | | protein1 | AT4G14690 | 0.0 | ELIP2 | Chlorophyll A-B binding |
| | | | | | | | | family protein |
| 22 | Zm00001d0 | 14.6 | | Photosystem | AT1G55670 | 0.6 | PSAG | photosystem I subunit G |
| | 05996 | | | I reaction | | | | |
| | | | | center subunit V | | | | |
| 23 | Zm00001d0 | 13.4 | pspb1 | photosystem | AT1G06680 | 2.6 | PSBP-1 | OE23, OXYGEN EVOLVING |
| | 07857 | | | II oxygen | | | | COMPLEX SUBUNIT 23 |
| | | | | evolving | | | | KDA, OEE2, OXYGEN- |
| | | | | polypeptide1 | | | | EVOLVING ENHANCER |
| | | | | | | | | PROTEIN 2, PSII- |
| | | | | | | | | P, PHOTOSYSTEM II |
| | | | | | | | | SUBUNIT P |
| 24 | Zm00001d0 | 12.6 | | Serine/threonine- | AT4G38470 | 4.9 | STY46 | ACT-like protein tyrosine |
| | 06267 | | | protein kinase | | | | kinase family protein |
| | | | | STY46 | | | | |
| 25 | Zm00001d0 | 12.5 | | cytochrome P450 | AT2G46660 | 1.8 | CYP78A6 | EOD3, enhancer of da1-1 |
| | 06193 | | | family 78 subfamily A | | | | |
| | | | | polypeptide 8 | | | | |
| 26 | Zm00001d0 | 12.3 | | Protein LURP1 | AT1G33840 | 1.3 | | LURP-one-like protein |
| | 05193 | | | | | | | (DUF567) |
| 27 | Zm00001d0 | 12.0 | IDP2449 | Gamma- | AT4G39640 | 1.0 | GGT1 | gamma-glutamyl |
| | 03446 | | | glutamyltrans- peptidase | | | | transpeptidase 1 |
| | | | | 1 | | | | |
| 28 | Zm00001d0 | 11.7 | | Probable alpha- | AT5G13980 | 1.6 | | Glycosyl hydrolase family 38 |
| | 07383 | | | mannosidase | | | | protein |
| 29 | Zm00001d0 | 8.6 | cl11315_1a | Protein disulfide- | AT1G75690 | 0.8 | LQY1 | DnaJ/Hsp40 cysteine-rich |
| | 03459 | | | isomerase LQY1 | | | | domain superfamily protein |
| | | | | chloroplastic | | | | |
| 30 | Zm00001d0 | 8.1 | | Protein kinase Kelch | AT2G44130 | 1.2 | KFB39 | Galactose oxidase/kelch |
| | 07274 | | | repeat:Kelch | | | | repeat superfamily protein, |
| | | | | | | | | Kelch-domain-containing F- |
| | | | | | | | | box protein 39, KMD3, KISS |
| | | | | | | | | ME DEADLY 3 |
| 31 | Zm00001d0 | 7.5 | | UDP- | AT2G43840 | 2.5 | UGT74F1 | UDP-glycosyltransferase 74 |
| | 06140 | | | glycosyltransferase | | | | F1 |
| | | | | 74B1 | AT2G43820 | 0.6 | UGT74F2 | ATSAGT1, Arabidopsis |
| | | | | | | | | thaliana salicylic acid |
| | | | | | | | | glucosyltransferase l |
| 32 | Zm00001d0 | 7.1 | pza03240 | Proline oxidase | AT3G30775 | 4.5 | ERD5 | AT- |
| | 29853 | | | | | | | POX, ATPDH, ATPOX, |
| | | | | | | | | ARABIDOPSIS |
| | | | | | | | | THALIANA PROLINE |
| | | | | | | | | OXIDASE, PDH1, proline |
| | | | | | | | | dehydrogenase |
| | | | | | | | | 1, PRO1, PRODH, PROLINE |
| | | | | | | | | DEHYDROGENASE |
| 33 | Zm00001d0 | 5.9 | pco112665 | Bifunctional protein | AT3G12290 | 0.8 | | Amino acid dehydrogenase |
| | 10867 | | | FolD 2 | | | | family protein |
| 34 | Zm00001d0 | 5.7 | | Oxygen-evolving | AT4G21280 | 1.5 | PSBQA | PSBQ-1, PHOTOSYSTEM II |
| | 06540 | | | enhancer protein 3-1 | | | | SUBUNIT Q- |
| | | | | | | | | 1, PSBQ, PHOTOSYSTEM II |
| | | | | | | | | SUBUNIT Q |
| | | | | | AT4G05180 | 1.7 | PS BQ-2 | PS BQ, PHOTOSYSTEM II |
| | | | | | | | | SUBUNIT Q, PSII-Q |
| 35 | Zm00001d0 | 5.7 | | 3′-5′ exonuclease | AT2G25910 | 2.9 | | 3′-5′ exonuclease domain- |
| | 11188 | | | domain-containing | | | | containing protein/K |
| | | | | protein/K homology | | | | homology domain-containing |
| | | | | domain-containing | | | | protein/KH domain- |
| | | | | protein/KH domain- | | | | containing protein |
| | | | | containing protein | | | | |
| 36 | Zm00001d0 | 5.4 | | Mitochondrial | AT1G79900 | 2.8 | BAC2 | Mitochondrial substrate |
| | 08974 | | | arginine | | | | carrier family protein |
| | | | | transporter BAC2 | | | | |
| 37 | Zm00001d0 | 5.4 | Ihca1 | light harvesting | AT3G61470 | 0.5 | LHCA2 | photosystem l light |
| | 06663 | | | complex A1 | | | | harvesting complex protein |
| 38 | Zm00001d0 | 4.7 | | Serinc-domain | AT4G13345 | 1.1 | MEE55 | Serinc-domain containing |
| | 08772 | | | containing serine and | | | | serine and sphingolipid |
| | | | | sphingolipid biosynthesis | | | | biosynthesis protein |
| | | | | protein | | | | |
| 39 | Zm00001d0 | 4.2 | | L-type lectin-domain | AT4G02420 | 0.2 | LecRK- | Concanavalin A-like lectin |
| | 24637 | | | containing receptor | | | IV.4, L-type | protein kinase family protein |
| | | | | kinase V.9 | | | lectin | |
| | | | | | | | receptor | |
| | | | | | | | kinase IV.4 | |
| 40 | Zm00001d0 | 4.1 | umc1272 | Probable amino acid | AT5G23810 | 0.7 | AAP7 | amino acid permease 7 |
| | 25665 | | | permease 7 | | | | |
| 41 | Zm00001d0 | 4.0 | hct6 | hydroxycinnamoyl- | AT5G48930 | 5.1 | HCT | hydroxycinnamoyl-CoA |
| | 17186 | | | transferase6 | | | | shikimate/quinate |
| | | | | | | | | hydroxycinnamoyl |
| | | | | | | | | transferase |
| 42 | Zm00001d0 | 3.9 | | Tetratricopeptide | AT4G10840 | 0.9 | KLCR1 | Tetratricopeptide repeat |
| | 14961 | | | repeat | | | | (TPR)-like superfamily |
| | | | | (TPR)-like | | | | protein |
| | | | | superfamily | | | | |
| | | | | protein | | | | |
| 43 | Zm00001d0 | 3.7 | AY109733 | 40S ribosomal protein | AT4G09800 | 8.2 | RPS18C | S18 ribosomal protein |
| | 13086 | | | S18 | | | | |
| 44 | Zm00001d0 | 3.7 | | UDP- | AT2G43840 | 2.5 | UGT74F1 | UDP-glycosyltransferase 74 |
| | 06137 | | | glycosyltransferase | | | | F1 |
| | | | | 74B1 | AT2G43820 | 0.6 | UGT74F2 | ATSAGT1, Arabidopsis |
| | | | | | | | | thaliana salicylic acid |
| | | | | | | | | glucosyltransferase |
| | | | | | | | | 1, GT, SAGT1, salicylic acid |
| | | | | | | | | glucosyltransferase |
| | | | | | | | | l, SGTl, UDP-glucose: salicylic |
| | | | | | | | | acid glucosyltransferase l |
| 45 | Zm00001d0 | 3.6 | mlkp3 | Maize LINC KASH | AT3G13360 | 3.7 | WIP3 | WPP domain interacting |
| | 05997 | | | AtWIP-like3 | | | | protein 3 |
| 46 | Zm00001d0 | 3.5 | lhcb6 | light harvesting | AT1G15820 | 3.1 | LHCB6 | CP24 |
| | 26599 | | | chlorophyll a/b binding | | | | |
| | | | | protein6 | | | | |
| 47 | Zm00001d0 | 3.4 | TIDP2961 | Auxin-responsive | AT1G29450 | 2.6 | SAUR64 | SMALL AUXIN |
| | 06274 | | | protein | | | | UPREGULATED |
| | | | | | | | | RNA 64 |
| | | | | SAUR61 | AT1G29510 | 1.4 | SAUR68 | SMALL AUXIN |
| | | | | | | | | UPREGULATED |
| | | | | | | | | RNA 68 |
| | | | | | AT1G29500 | 2.1 | SAUR66 | SMALL AUXIN |
| | | | | | | | | UPREGULATED |
| | | | | | | | | RNA 66 |
| 48 | Zm00001d0 | 3.4 | | Thioredoxin-like | AT1G76080 | 1.3 | CDSP32 | ATCDSP32, ARABIDOPSIS |
| | 21334 | | | protein | | | | THALIANA CHLOROPLASTIC |
| | | | | CDSP32 | | | | DROUGHT-INDUCED STRESS |
| | | | | chloroplastic | | | | PROTEIN OF 32 KD |
| 49 | Zm00001d0 | 3.3 | | DEAD-box ATP- | AT5G62190 | 2.4 | PRH75 | DEAD box RNA helicase |
| | 06160 | | | dependent RNA | | | | (PRH75) |
| | | | | helicase 7 | | | | |
| 50 | Zm00001d0 | 3.0 | | Sm-like protein | AT5G48870 | 2.9 | SADI | AtLSM5, AtSAD1, LSM5, SM- |
| | 38088 | | | LSM5 | | | | like 5 |
| 51 | Zm00001d0 | 3.0 | | Ultraviolet- | AT2G06520 | 0.7 | PSBX | photosystem II subunit X |
| | 08681 | | | B-repressible | | | | |
| | | | | protein | | | | |
| 52 | Zm00001d0 | 2.9 | fdh1 | formaldehyde | AT5G43940 | 0.7 | HOT5 | ADH2, ALCOHOL |
| | 18468 | | | dehydrogenase | | | | DEHYDROGENASE |
| | | | | homolog1 | | | | 2, ATGSNOR1, GSNOR,S- |
| | | | | | | | | NITROSOGLUTATHIONE |
| | | | | | | | | REDUCTASE, PAR2, PARAQUAT |
| | | | | | | | | RESISTANT 2 |
| 53 | Zm00001d0 | 2.9 | mkkk27 | MAP kinase kinase | AT5G28080 | 0.7 | WNK9 | Protein kinase superfamily |
| | 06644 | | | kinase27 | | | | protein |
| 54 | Zm00001d0 | 2.9 | IhcblO | light harvesting | AT2G34430 | 0.4 | LHB1B1 | LHCB1.4, LIGHT- |
| | 11285 | | | chlorophyll a/b | | | | HARVESTING |
| | | | | binding | | | | CHLOROPHYLL-PROTEIN |
| | | | | | | | | COMPLEX II SUBUNIT Bl |
| | | | | protein10 | AT2G34420 | 0.6 | LHB1B2 | LHCB1.5, PHOTOSYSTEM II |
| | | | | | | | | LIGHT HARVESTING |
| | | | | | | | | COMPLEX GENE 1.5 |
| | | | | | AT1G29910 | 0.4 | CAB3 | AB180, LHCB1.2, LIGHT |
| | | | | | | | | HARVESTING CHLOROPHYLL |
| | | | | | | | | A/B BINDING PROTEIN 1.2 |
| 55 | Zm00001d0 | 2.9 | | Serine | AT3G17180 | 0.5 | scpl33 | serine carboxypeptidase-like |
| | 09178 | | | carboxypeptidase- like 33 | | | | 33 |
| 56 | Zm00001d0 | 2.8 | pco070301 | MtN19-like protein | AT5G61820 | 1.4 | | stress up-regulated Nod 19 |
| | 31677 | | | | | | | protein |
| 57 | Zm00001d0 | 2.8 | | Rhodanese-like | AT4G01050 | 2.3 | TROL | thylakoid rhodanese-like |
| | 16100 | | | domain- | | | | protein |
| | | | | containing protein 4 | | | | |
| | | | | chloroplastic | | | | |
| 58 | Zm00001d0 | 2.6 | | Phospholipase A1- | AT2G30550 | 0.6 | DALL3 | alpha/beta-Hydrolases |
| | 10463 | | | Igamma1 | | | | superfamily protein, DAD1- |
| | | | | chloroplastic | | | | Like Lipase 3 |
| 59 | Zm00001d0 | 2.5 | stcl | sesquiterpene | AT4G20230 | 0.3 | | terpenoid synthase |
| | 45054 | | | cyclase1 | | | | superfamily protein |
| 60 | Zm00001d0 | 2.5 | nrt5 | nitrate transports | AT1G12940 | 7.9 | NRT2.5 | ATNRT2.5, nitrate |
| | 11679 | | | | | | | transporter2.5 |
| 61 | Zm00001d0 | 2.5 | npi447a | agal1; alpha- | AT5G08370 | 9.0 | AGAL2 | AtAGAL2, alpha-galactosidase |
| | 32605 | | | galactosidase1: Entrez | | | | 2 |
| | | | | Gene relates to alpha- | | | | |
| | | | | galactosidase 1 (AGAL) | | | | |
| | | | | of Arabidopsis | | | | |
| 62 | Zm00001d0 | 2.5 | oec33 | oxygen evolving | AT5G66570 | 0.4 | PSBO1 | MSP-1, MANGANESE- |
| | 36535 | | | complex, 33 kDa | | | | STABILIZING PROTEIN |
| | | | | subunit | | | | 1, OE33, OXYGEN EVOLVING |
| | | | | | | | | COMPLEX 33 KILODALTON |
| | | | | | | | | PROTEIN, OEE1, 33 KDA |
| | | | | | | | | OXYGEN EVOLVING |
| | | | | | | | | POLYPEPTIDE |
| | | | | | | | | 1, OEE33, OXYGEN EVOLVING |
| | | | | | | | | ENHANCER PROTEIN |
| | | | | | | | | 33, PSBO-1, PS II OXYGEN- |
| | | | | | | | | EVOLVING COMPLEX 1 |
| | | | | | AT3G50820 | 1.7 | PSBO2 | OEC33, OXYGEN EVOLVING |
| | | | | | | | | COMPLEX SUBUNIT 33 |
| | | | | | | | | KDA, PSBO-2, PHOTOSYSTEM |
| | | | | | | | | II SUBUNIT O-2 |
| 63 | Zm00001d0 | 2.4 | pco129777 | Phospho- | AT1G48600 | 0.6 | PMEAMT | AtPMEAMT |
| | 11642 | | b | ethanolamine | | | | |
| | | | | N-methyl- | | | | |
| | | | | transferase 3 | | | | |
| 64 | Zm00001d0 | 2.4 | | cytochrome P450 | AT3G14690 | 0.7 | CYP72A15 | cytochrome P450, family 72, |
| | 11418 | | | family 72 subfamily | | | | subfamily A, polypeptide 15 |
| | | | | A polypeptide 8 | | | | |
| 65 | Zm00001d0 | 2.4 | | Agmatine deiminase | AT5G08170 | 2.5 | EMB1873 | ATAIH, AGMATINE |
| | 25644 | | | | | | | IMINOHYDROLASE |
| 66 | Zm00001d0 | 2.4 | | Syntaxin-81 | AT1G51740 | 2.4 | SYP81 | ATSYP81, ATUFE1, |
| | 25915 | | | | | | | ARABIDOPSIS |
| | | | | | | | | THALIANA ORTHOLOG OF |
| | | | | | | | | YEAST UFE1 (UNKNOWN |
| | | | | | | | | FUNCTION-ESSENTIAL |
| | | | | | | | | 1), UFE1, ORTHOLOG OF |
| | | | | | | | | YEAST UFE1 (UNKNOWN |
| | | | | | | | | FUNCTION-ESSENTIAL 1), |
| | | | | | | | | Rhodanese/Cell cycle control |
| | | | | | | | | phosphatase superfamily |
| | | | | | | | | protein |
| 67 | Zm00001d0 | 2.4 | | Rhodanese-like | AT2G42220 | 0.7 | | |
| | 19899 | | | domain- | | | | |
| | | | | containing protein 9 | | | | |
| | | | | chloroplastic | | | | |
| 68 | Zm00001d0 | 2.3 | | Vacuolar-sorting | AT2G14740 | 0.4 | VSR3 | ATVSR3, vaculolar sorting |
| | 14303 | | | receptor 4 | | | | receptor 3, BP80-2; 2, binding |
| | | | | | | | | protein of 80 kDa |
| | | | | | | | | 2; 2, VSR2; 2, VACUOLAR |
| | | | | | | | | SORTING RECEPTOR 2; 2 |
| 69 | Zm00001d0 | 2.3 | amo1 | amine oxidase1 | AT4G12290 | 1.5 | | Copper amine oxidase family |
| | 25103 | | | | | | | protein |
| 70 | Zm00001d0 | 2.3 | | Photosystem | AT1G31330 | 1.4 | PSAF | photosystem I subunit F |
| | 13146 | | | I reaction | | | | |
| | | | | center subunit III | | | | |
| | | | | chloroplastic | | | | |
| 71 | Zm00001d0 | 2.3 | psah1 | photosystem I H | AT3G16140 | 0.3 | PSAH-1 | photosystem I subunit H-1 |
| | 38984 | | | subunit1 | | | | |
| 72 | Zm00001d0 | 2.3 | atg18d | autophagy18d | AT3G56440 | 0.3 | ATG18D | ATATG18D, homolog of yeast |
| | 08691 | | | | | | | autophagy 18 (ATG18) D |
| 73 | Zm00001d0 | 2.1 | Iox5 | lipoxygenase5 | AT3G22400 | 4.6 | LOX5 | PLAT/LH2 domain-containing |
| | 13493 | | | | | | | lipoxygenase family protein |
| 74 | Zm00001d0 | 2.1 | cdc2 | cell division control | AT3G48750 | 1.9 | CDC2 | CDC2A, CDC2AAT, CDK2, |
| | 27373 | | | protein homolog2 | | | | CDKA 1, CDKA; 1 |
| 75 | Zm00001d0 | 2.1 | Iox1 | lipoxygenase1 | AT3G22400 | 4.6 | LOX5 | PLAT/LH2 domain-containing |
| | 42541 | | | | | | | lipoxygenase family protein |
| 76 | Zm00001d0 | 2.1 | psb29 | photosystem II | AT3G08940 | 3.8 | LHCB4.2 | light harvesting complex |
| | 21763 | | | subunit29 | | | | photosystem II |
| | | | | | AT5G01530 | 2.0 | LHCB4.1 | light harvesting complex |
| | | | | | | | | photosystem II |
| 77 | Zm00001d0 | 2.1 | hex3 | hexokinase3 | AT2G19860 | 3.3 | HXK2 | ATHXK2, ARABIDOPSIS |
| | 10796 | | | | | | | THALIANA HEXOKINASE 2 |
| 78 | Zm00001d0 | 2.1 | | Vacuolar protein | AT3G49645 | 1.4 | FAD- | |
| | 34796 | | | sorting- | | | binding | |
| | | | | associated protein 9A | | | protein | |
| 79 | Zm00001d0 | 2.0 | | Peptidyl-prolyl | AT3G25220 | 6.2 | FKBP15-1 | FK506-binding protein 15 kD- |
| | 21021 | | | cis-trans | | | | 1 |
| | | | | isomerase | | | | |
| 80 | Zm00001d0 | 2.0 | | S-adenosyl-L- | AT2G41380 | 1.7 | | S-adenosyl-L-methionine- |
| | 09084 | | | methionine- | | | | dependent |
| | | | | dependent | | | | methyltransferases |
| | | | | methyltransferases | | | | superfamily protein |
| | | | | superfamily protein | | | | |
| 81 | Zm00001d0 | 1.9 | | alpha/beta-Hydrolases | AT5G38220 | 9.1 | | alpha/beta-Hydrolases |
| | 11624 | | | superfamily protein | | | | superfamily protein |
| 82 | Zm00001d0 | 1.9 | peamt2 | Phosphoethanolamine | AT1G48600 | 0.6 | PMEAMT | AtPMEAMT |
| | 38891 | | | N-methyltransferase 3 | | | | |
| 83 | Zm00001d0 | 1.8 | | Methyl-CpG-binding | AT5G52230 | 1.1 | MBD13 | methyl-CPG-binding domain |
| | 24306 | | | domain-containing | | | | protein 13 |
| | | | | protein 13 | | | | |
| 84 | Zm00001d0 | 1.8 | | chaperone protein | AT5G43260 | 1.4 | | chaperone protein dnaJ-like |
| | 16561 | | | dnaJ-related | | | | protein |
| 85 | Zm00001d0 | 1.8 | sqd1 | sulfolipid | AT4G33030 | 9.8 | SQD1 | sulfoquinovosyldiacylglycerol |
| | 09967 | | | biosynthesis1 | | | | 1 |
| 86 | Zm00001d0 | 1.8 | mpk4 | MAP kinase4 | AT4G01370 | 1.3 | MPK4 | ATMPK4, MAP kinase |
| | 24568 | | | | | | | 4, MAPK4 |
| | | | | | AT1G01560 | 1.0 | MPK11 | ATMPK11, MAP kinase 11 |
| 87 | Zm00001d0 | 1.7 | | Phospho-2-dehydro- | AT1G22410 | 4.3 | | Class-11 DAHP synthetase |
| | 06900 | | | 3-deoxyheptonate | | | | family protein |
| | | | | aldolase 2 chloroplastic | | | | |
| 88 | Zm00001d0 | 1.7 | | actin binding protein | AT1G52080 | 9.7 | AR791 | actin binding protein family |
| | 37695 | | | family | | | | |
| 89 | Zm00001d0 | 1.7 | rl1 | radialis homolog1 | AT1G19510 | 2.0 | | |
| | 39118 | | | | AT4G39250 | 6.9 | | |
| 90 | Zm00001d0 | 1.5 | | Histidine-containing | AT3G16360 | 4.1 | AHP4 | HPT phosphotransmitter 4 |
| | 10791 | | | phosphotransfer | | | | |
| | | | | protein 4 | | | | |
| 91 | Zm00001d0 | 1.5 | | Protein | AT4G11910 | 3.6 | NYE2, | STAY-GREEN-like protein |
| | 06211 | | | STAY-GREEN 1 | | | NONY | |
| | | | | chloroplastic | | | ELLOWING | |
| | | | | | | | 2, SGR2, | |
| | | | | | | | STAY- | |
| | | | | | | | GREEN 2 | |
| | | | | | AT4G22920 | 0.6 | NYE1 | ATNYE1, NON-YELLOWING |
| | | | | | | | | 1, SGR1, STAY-GREEN |
| | | | | | | | | 1, SGR, STAY-GREEN |
| 92 | Zm00001d0 | 1.5 | | Histone deacetylase | AT2G45640 | 6.3 | SAP18 | ATSAP18, SIN3 ASSOCIATED |
| | 15058 | | | complex subunit | | | | POLYPEPTIDE 18 |
| | | | | SAP18 | | | | |
| 93 | Zm00001d0 | 1.5 | | Probable | AT3G23790 | 7.7 | AAE16 | AMP-dependent synthetase |
| | 34832 | | | acyl-activating | | | | and ligase family protein |
| | | | | enzyme 16 | | | | |
| | | | | chloroplastic | | | | |
| 94 | Zm00001d0 | 1.5 | | Chlorophyll | AT3G47470 | 1.0 | LHCA4 | CAB4 |
| | 50403 | | | a-b binding | | | | |
| | | | | protein 4 | | | | |
| 95 | Zm00001d0 | 1.4 | mrpa10 | multidrug resistance | AT3G59140 | 4.1 | ABCC10 | ATMRP14, multidrug |
| | 31447 | | | protein associated10 | | | | resistance-associated protein |
| | | | | | | | | 14, MRP14, multidrug |
| | | | | | | | | resistance-associated protein |
| | | | | | | | | 14 |
| 96 | Zm00001d0 | 1.4 | | Photosystem | AT1G55670 | 0.6 | PSAG | photosystem I subunit G |
| | 20877 | | | I reaction | | | | |
| | | | | center subunit V | | | | |
| | | | | chloroplastic | | | | |
| 97 | Zm00001d0 | 1.3 | | Nuclear pore | AT3G15970 | 3.1 | NUP50 | (Nucleoporin 50 kDa) protein |
| | 43757 | | | complex | | | | |
| | | | | protein NUP50A | | | | |
| 98 | Zm00001d0 | 1.3 | gpm930 | Photosystem | AT4G28750 | 0.8 | PSAE-1 | Photosystem I reaction |
| | 19518 | | | I reaction | | | | centre subunit IV/PsaE |
| | | | | center subunit IV A | | | | protein |
| | | | | | AT2G20260 | 0.4 | PSAE-2 | photosystem I subunit E-2 |
| 99 | Zm00001d0 | 1.3 | IDP755 | D111/G-patch | AT1G63980 | 12.5 | | D111/G-patch domain- |
| | 27444 | | | domain- | | | | containing protein |
| | | | | containing protein | | | | |
| 100 | Zm00001d0 | 1.3 | | GTP-binding protein | AT5G57960 | 2.3 | HfIx | GTP-binding protein, HfIX |
| | 48944 | | | hfIX | | | | |
| 101 | Zm00001d0 | 1.3 | | Glucomannan | AT5G22740 | 1.1 | CSLA02 | ATCSLA02, ARABIDOPSIS |
| | 53696 | | | 4-beta- | | | | THALIANA CELLULOSE |
| | | | | mannosyl- | | | | SYNTHASE-LIKE |
| | | | | transferase 2 | | | | A02, ATCSLA2, ARABIDOPSIS |
| | | | | | | | | THALIANA CELLULOSE |
| | | | | | | | | SYNTHASE-LIKE |
| | | | | | | | | A2, CSLA2, CELLULOSE |
| | | | | | | | | SYNTHASE-LIKE A 2 |
| 102 | Zm00001d0 | 1.3 | umc1383 | lhcb9; light | AT2G05070 | 4.2 | LHCB2.2 | LHCB2, LIGHT-HARVESTING |
| | 33132 | | | harvesting | | | | CHLOROPHYLL B-BINDING 2 |
| | | | | chlorophyll binding | | | | |
| | | | | protein9: cDNA | | | | |
| | | | | sequence is a classll | | | | |
| | | | | Ihcb, unlike previously | | | | |
| | | | | characterized lhcb genes | | | | |
| | | | | which are class1 | | | | |
| | | | | (Viret et al 1993) | | | | |
| 103 | Zm00001d0 | 1.2 | lhcb2 | light harvesting | AT2G34430 | 0.4 | LHB1B1 | LHCB1.4, |
| | 21435 | | | chlorophyll a/b | | | | LIGHT-HARVESTING |
| | | | | binding | | | | CHLOROPHYLL-PROTEIN |
| | | | | protein2 | | | | COMPLEX II SUBUNIT B1 |
| | | | | | AT2G34420 | 0.6 | LHB1B2 | LHCB1.5, PHOTOSYSTEM II |
| | | | | | | | | LIGHT HARVESTING |
| | | | | | | | | COMPLEX GENE 1.5 |
| | | | | | AT1G29910 | 0.4 | CAB3 | AB180, LHCB1.2, LIGHT |
| | | | | | | | | HARVESTING CHLOROPHYLL |
| | | | | | | | | A/B BINDING PROTEIN 1.2 |
| 104 | Zm00001d0 | 1.2 | | Nuclear transport | AT5G04830 | 1.2 | | Nuclear transport factor 2 |
| | 11799 | | | factor | | | | (NTF2) family protein |
| | | | | 2 (NTF2) family | | | | |
| | | | | protein | | | | |
| 105 | Zm00001d0 | 1.2 | | Pollen Ole e 1 | AT5G15780 | 1.1 | | Pollen Ole e 1 allergen and |
| | 52518 | | | allergen | | | | extensin family protein |
| | | | | and extensin family | | | | |
| | | | | protein | | | | |
| 106 | Zm00001d0 | 1.1 | | Chlorophyll a-b | AT3G61470 | 0.5 | LHCA2 | photosystem I light |
| | 21906 | | | binding protein | | | | harvesting complex protein |
| 107 | Zm00001d0 | 1.1 | pip1b | plasma membrane | AT4G23400 | 0.6 | PIP1; 5 | PIP1D |
| | 17526 | | | intrinsic protein1 | AT1G01620 | 0.9 | PIP1C | PIP1; 3, PLASMA MEMBRANE |
| | | | | | | | | INTRINSIC PROTEIN 1; 3, |
| | | | | | | | | TMP-B |
| 108 | Zm00001d0 | 1.1 | | D-xylose-proton | AT5G17010 | 2.8 | | Major facilitator superfamily |
| | 14435 | | | symporter-like l | | | | protein |
| 109 | Zm00001d0 | 1.1 | psad1 | photosystem I | AT4G02770 | 0.5 | PSAD-1 | photosystem I subunit D-1 |
| | 13039 | | | subunit d1 | | | | |
| 110 | Zm00001d0 | 1.1 | | photosystem II light | AT2G34430 | 0.4 | LHB1B1 | LHCB1.4, |
| | 44401 | | | harvesting complex | | | | LIGHT-HARVESTING |
| | | | | gene B1B2 | | | | CHLOROPHYLL-PROTEIN |
| | | | | | | | | COMPLEX II SUBUNIT B1 |
| | | | | | AT2G34420 | 0.6 | LHB1B2 | LHCB1.5, PHOTOSYSTEM II |
| | | | | | | | | LIGHT HARVESTING |
| | | | | | | | | COMPLEX GENE 1.5 |
| | | | | | AT1G29910 | 0.4 | CAB3 | AB180, LHCB1.2, LIGHT |
| | | | | | | | | HARVESTING CHLOROPHYLL |
| | | | | | | | | A/B BINDING PROTEIN 1.2 |
| ill | Zm00001d0 | 1.1 | | Phospho-2-dehydro- | AT1G22410 | 4.3 | | Class-II DAHP synthetase |
| | 22181 | | | 3-deoxyheptonate | | | | family protein |
| | | | | aldolase 1 | | | | |
| 112 | Zm00001d0 | 1.1 | AY111834 | Cytochrome P450 | AT2G46660 | 1.8 | CYP78A6 | EOD3, enhancer of da1-1 |
| | 32042 | | | CYP78A53 | | | | |
| 113 | Zm00001d0 | 1.1 | elip2 | early light inducible | AT3G22840 | 0.2 | ELIP1 | ELIP |
| | 18940 | | | protein2 | AT4G14690 | 0.0 | ELIP2 | Chlorophyll A-B binding |
| | | | | | | | | family protein |
| 114 | Zm00001d0 | 1.1 | | Chlorophyll | AT3G47470 | 1.0 | LHCA4 | CAB4 |
| | 32197 | | | a-b binding | | | | |
| | | | | protein 4 | | | | |
| | | | | chloroplastic | | | | |
| 115 | Zm00001d0 | 1.1 | d9 | dwarf plant9 | AT1G14920 | 0.5 | | |
| | 13465 | | | | | | | |
| 116 | Zm00001d0 | 1.1 | | hydroxyproline-rich | AT2G39050 | 1.8 | EULS3 | ArathEULS3 |
| | 40190 | | | glycoprotein family | | | | |
| | | | | protein | | | | |
| 117 | Zm00001d0 | 1.1 | | 405 ribosomal | AT4G09800 | 8.2 | RPS18C | S18 ribosomal protein |
| | 34422 | | | protein 518 | | | | |
| 118 | Zm00001d0 | 1.0 | | Transcription factor | AT3G20640 | 3.6 | | |
| | 43248 | | | bHLH112 | | | | |
| 119 | Zm00001d0 | 1.0 | alia1 | allantoinase1 | AT4G04955 | 15.7 | ALN | ATALN, allantoinase |
| | 26635 | | | | | | | |
| 120 | Zm00001d0 | 1.0 | cncr1 | cinnamoyl CoA | AT1G80820 | 4.6 | CCR2 | ATCCR2 |
| | 32152 | | | reductase1 | | | | |
| 121 | Zm00001d0 | 1.0 | | BTB/POZ domain- | AT1G55760 | 10.5 | SIBP1 | BTB/POZ domain-containing |
| | 52837 | | | containing protein | | | | protein |
| 122 | Zm00001d0 | 1.0 | pspb2 | photosystem | AT1G06680 | 2.6 | PSBP-1 | OE23, OXYGEN EVOLVING |
| | 18779 | | | II oxygen | | | | COMPLEX SUBUNIT 23 |
| | | | | evolving | | | | KDA, OEE2, OXYGEN- |
| | | | | polypeptide2 | | | | EVOLVING ENHANCER |
| | | | | | | | | PROTEIN 2, PSII- |
| | | | | | | | | P, PHOTOSYSTEM II SUBUNIT |
| | | | | | | | | P |
| 123 | Zm00001d0 | 1.0 | | Alanine-glyoxylate | AT4G39660 | 14.4 | AGT2 | alanine: glyoxylate |
| | 27861 | | | aminotransferase 2 | | | | aminotransferase 2 |
| | | | | homolog 1 | | | | |
| | | | | mitochondrial | | | | |
| 124 | Zm00001d0 | 0.9 | | Serine/threonine- | AT1G65800 | 0.8 | RK2 | ARK2, receptor kinase |
| | 12609 | | | protein kinase | | | | 2, AtARK2 |
| 125 | Zm00001d0 | 0.9 | | Serine/threonine- | AT4G21380 | 0.3 | RK3 | ARK3, receptor kinase 3 |
| | 12609 | | | protein kinase | AT1G65790 | 0.1 | RK1 | ARK1, receptor kinase 1 |
| 126 | Zm00001d0 | 0.9 | | Encodes a protein | AT1G66480 | 11.5 | | plastid movement impaired 2 |
| | 32233 | | | whose expression | | | | |
| | | | | is responsive | | | | |
| | | | | to nematode infection. | | | | |
| 127 | Zm00001d0 | 0.9 | | Putative | AT5G60900 | 0.1 | RLK1 | receptor-like protein kinase 1 |
| | 25035 | | | D-mannose | | | | |
| | | | | binding lectin family | | | | |
| | | | | receptor-like protein | | | | |
| | | | | kinase | | | | |
| 128 | Zm00001d0 | 0.9 | | ubiquitin-associated | AT1G04850 | 5.0 | | ubiquitin-associated |
| | 50551 | | | (UBA)/TS-N | | | | (UBA)/TS-N domain- |
| | | | | domain- | | | | containing protein |
| | | | | containing protein | | | | |
| 129 | Zm00001d0 | 0.8 | | Peptide transporter | AT1G52190 | 0.4 | AtNPF1.2, | Major facilitator superfamily |
| | 43374 | | | PTR2 | | | NP | protein |
| | | | | | | | F1.2, NRT1/ | |
| | | | | | | | PTR family | |
| | | | | | | | 1.2, | |
| | | | | | | | NRT1.11 | |
| 130 | Zm00001d0 | 0.8 | psan1 | photosystem I N | AT5G64040 | 0.8 | PSAN | photosystem I reaction |
| | 41819 | | | subunit1 | | | | center subunit PSI-N, |
| | | | | | | | | chloroplast, putative/PSI-N, |
| | | | | | | | | putative (PSAN) |
| 131 | Zm00001d0 | 0.8 | pba1 | PBA1 homolog1 | AT4G01150 | 0.8 | CURT1A | CURVATURE THYLAKOID |
| | 27456 | | | | | | | 1A-like protein |
| 132 | Zm00001d0 | 0.8 | psan2 | photosystem I N | AT5G64040 | 0.8 | PSAN | photosystem I reaction |
| | 23713 | | | subunit2 | | | | center subunit PSI-N, |
| | | | | | | | | chloroplast, putative/PSI-N, |
| | | | | | | | | putative (PSAN) |
| 133 | Zm00001d0 | 0.8 | TIDP3460 | cytochrome | AT1G57750 | 2.5 | CYP96A15 | MAH1, MID-CHAIN ALKANE |
| | 27601 | | | P450 family | | | | HYDROXYLASE 1 |
| | | | | 96 subfamily A | | | | |
| | | | | polypeptide 1 | | | | |
| 134 | Zm00001d0 | 0.8 | | Zn-dependent | AT4G33540 | 0.6 | | met allo-beta-lactamase |
| | 51842 | | | hydrolase%2C | | | | family protein |
| | | | | including | | | | |
| | | | | glyoxylase | | | | |
| 135 | Zm00001d0 | 0.8 | | Peroxiredoxin-5 | AT3G52960 | 1.1 | | Thioredoxin superfamily |
| | 46682 | | | | | | | protein |
| 136 | Zm00001d0 | 0.8 | ago101 | argonaute101 | AT5G43810 | 1.8 | AGO10 | PNH, PINHEAD, ZLL, |
| | 46438 | | | | | | | ZWILLE |
| 137 | Zm00001d0 | 0.8 | | alpha/beta- | AT4G39955 | 9.1 | | alpha/beta-Hydrolases |
| | 22182 | | | Hydrolases | | | | superfamily protein |
| | | | | superfamily protein | | | | |
| 138 | Zm00001d0 | 0.8 | | Thioredoxin M1 | AT3G15360 | 1.2 | TRX-M4 | ATHM4, ATM4, ARABIDOPSIS |
| | 17379 | | | chloroplastic | | | | THIOREDOXIN M-TYPE 4 |
| 139 | Zm00001d0 | 0.8 | | SPIa/RYanodine | AT1G35470 | 15.3 | RanBPM | SPIa/RYanodine receptor |
| | 16825 | | | receptor | | | | (SPRY) domain-containing |
| | | | | (SPRY) domain- | | | | protein |
| | | | | containing protein | AT4G09340 | 1.5 | | SPIa/RYanodine receptor |
| | | | | | | | | (SPRY) domain-containing |
| | | | | | | | | protein |
| 140 | Zm00001d0 | 0.7 | | Phosphatase | AT1G17710 | 0.6 | PEPC1 | AtPEPC1, Arabidopsis thaliana |
| | 43621 | | | phospho1 | | | | phosphoethanolamine/phos |
| | | | | | | | | phocholine phosphatase 1 |
| 141 | Zm00001d0 | 0.7 | | UDP-glucuronic acid | AT5G59290 | 0.5 | UXS3 | ATUXS3 |
| | 47797 | | | decarboxylase 5 | | | | |
| 142 | Zm00001d0 | 0.7 | | Probable | AT1G79110 | 3.1 | BRG2 | zinc ion binding protein |
| | 33419 | | | BOI-related | | | | |
| | | | | E3 ubiquitin-protein | | | | |
| | | | | ligase 2 | | | | |
| 143 | Zm00001d0 | 0.7 | IDP518 | Chlorophyll | AT2G34430 | 0.4 | LHB1B1 | LHCB1.4, |
| | 44396 | | | a-b binding | | | | LIGHT-HARVESTING |
| | | | | protein 48% 2C | | | | CHLOROPHYLL-PROTEIN |
| | | | | chloroplastic | | | | COMPLEX II SUBUNIT B1 |
| | | | | | AT2G34420 | 0.6 | LHB1B2 | LHCB1.5, PHOTOSYSTEM II |
| | | | | | | | | LIGHT HARVESTING |
| | | | | | | | | COMPLEX GENE 1.5 |
| | | | | | AT1G2991O | 0.4 | CAB3 | AB180, LHCB1.2, LIGHT |
| | | | | | | | | HARVESTING CHLOROPHYLL |
| | | | | | | | | A/B BINDING PROTEIN 1.2 |
| 144 | Zm00001d0 | 0.7 | gst31 | glutathione | AT1G59700 | 0.4 | GSTU16 | ATGSTU16, glutathione S- |
| | 27557 | | | transferase31 | | | | transferase TAU 16 |
| | | | | | AT1G59670 | 0.6 | GSTU15 | ATGSTU15, glutathione S- |
| | | | | | | | | transferase TAU 15 |
| 145 | Zm00001d0 | 0.7 | pco123453 | S-adenosyl-L- | AT4G28830 | 1.4 | | S-adenosyl-L-methionine- |
| | 36274 | | | methionine- | | | | dependent |
| | | | | dependent | | | | methyltransferases |
| | | | | methyltransferases | | | | superfamily protein |
| | | | | superfamily protein | | | | |
| 146 | Zm00001d0 | 0.6 | idh1 | isocitrate | AT1G65930 | 0.8 | cICDH | cytosolic NADP+-dependent |
| | 11487 | | | dehydrogenase1 | | | | isocitrate dehydrogenase |
| 147 | Zm00001d0 | 0.6 | pip1e | plasma membrane | AT4G23400 | 0.6 | PIP1; 5 | PIP1D |
| | 51872 | | | intrinsic proteinl | AT1G01620 | 0.9 | PIP1C | PIP1; 3, PLASMA MEMBRANE |
| | | | | | | | | INTRINSIC PROTEIN 1; 3, |
| | | | | | | | | TMP-B |
| 148 | Zm00001d0 | 0.6 | | Putative leucine-rich | AT1G28440 | 0.5 | HSL1 | HAESA-like 1 |
| | 09029 | | | repeat receptor-like | | | | |
| | | | | protein kinase family | | | | |
| | | | | protein | | | | |
| 149 | Zm00001d0 | 0.6 | | Metallothionein-like | AT5G02380 | 1.9 | MT2B | metallothionein 2B |
| | 39914 | | | protein type 2 | | | | |
| 150 | Zm00001d0 | 0.6 | | Snf1-related kinase | AT1G80940 | 2.6 | | Snf1 kinase interactor-like |
| | 18364 | | | interacting protein SKI1 | | | | protein |
| 151 | Zm00001d0 | 0.6 | | ROTUNDIFOLIA | AT2G39705 | 0.6 | RTFL8 | DVL11, DEVIL 11 |
| | 28598 | | | like 8 | | | | |
| 152 | Zm00001d0 | 0.6 | | ATPase | AT4G28070 | 0.6 | | AFG1-like ATPase family |
| | 25892 | | | | | | | protein |
| 153 | Zm00001d0 | 0.6 | | Ultraviolet-B- | AT2G06520 | 0.7 | PSBX | photosystem II subunit X |
| | 39715 | | | repressible | | | | |
| | | | | protein | | | | |
| 154 | Zm00001d0 | 0.6 | | Protein LRP16 | AT2G40600 | 0.3 | | appr-1-p processing enzyme |
| | 29065 | | | | | | | family protein |
| 155 | Zm00001d0 | 0.6 | | Proline oxidase | AT3G30775 | 4.5 | ERD5 | AT- |
| | 47124 | | | | | | | POX, ATPDH, ATPOX, |
| | | | | | | | | ARABIDOPSIS |
| | | | | | | | | THALIANA PROLINE |
| | | | | | | | | OXIDASE, PDH1, proline |
| | | | | | | | | dehydrogenase |
| | | | | | | | | 1, PRO1, PRODH, PROLINE |
| | | | | | | | | DEHYDROGENASE |
| 156 | Zm00001d0 | 0.6 | | NAD(P)-linked | AT1G59950 | 1.1 | | NAD(P)-linked |
| | 28360 | | | oxidoreductase | | | | oxidoreductase superfamily |
| | | | | superfamily protein | | | | protein |
| 157 | Zm00001d0 | 0.6 | gdh1 | glutamic | AT3G03910 | 4.1 | GDH3 | glutamate dehydrogenase 3 |
| | 34420 | | | dehydrogenase1 | | | | |
| 158 | Zm00001d0 | 0.6 | | Putative calcium- | AT4G09570 | 0.7 | CPK4 | ATCPK4 |
| | 23560 | | | dependent protein | | | | |
| | | | | kinase family protein | | | | |
| 159 | Zm00001d0 | 0.5 | gpm345 | NAD(P)H | AT4G27270 | 1.1 | | Quinone reductase family |
| | 12607 | | | dehydrogenase | | | | protein |
| | | | | (quinone) FQR1 | | | | |
| 160 | Zm00001d0 | 0.5 | oec33b | oxygen-evolving | AT5G66570 | 0.4 | PSBO1 | MSP-1, MANGANESE- |
| | 14564 | | | complex 33 kda | | | | STABILIZING PROTEIN |
| | | | | protein b | | | | 1, OE33, OXYGEN EVOLVING |
| | | | | | | | | COMPLEX 33 KILODALTON |
| | | | | | | | | PROTEIN, OEE1, 33 KDA |
| | | | | | | | | OXYGEN EVOLVING |
| | | | | | | | | POLYPEPTIDE |
| | | | | | | | | 1, OEE33, OXYGEN EVOLVING |
| | | | | | | | | ENHANCER PROTEIN |
| | | | | | | | | 33, PSBO-1, PS II OXYGEN- |
| | | | | | | | | EVOLVING COMPLEX 1 |
| | | | | | AT3G50820 | 1.7 | PSBO2 | OEC33, OXYGEN EVOLVING |
| | | | | | | | | COMPLEX SUBUNIT 33 |
| | | | | | | | | KDA, PSBO-2, PHOTOSYSTEM |
| | | | | | | | | II SUBUNIT O-2 |
| 161 | Zm00001d0 | 0.5 | kch1 | potassium | AT2G26650 | 1.8 | KT1 | AKT1, K+ transporter |
| | 44056 | | | channel 1 | | | | 1, ATAKT1 |
| 162 | Zm00001d0 | 0.5 | | 3′-5′ exonuclease | AT2G25910 | 2.9 | | 3′-5′ exonuclease domain- |
| | 44243 | | | domain-containing | | | | containing protein/K |
| | | | | protein/K homology | | | | homology domain-containing |
| | | | | domain-containing | | | | protein/KH domain- |
| | | | | protein/KH domain- | | | | containing protein |
| | | | | containing protein | | | | |
| 163 | Zm00001d0 | 0.5 | abh3 | abscisic acid 8′- | AT3G19270 | 0.5 | CYP707A4 | cytochrome P450, family |
| | 50021 | | | hydroxylase3 | | | | 707, subfamily A, |
| | | | | | | | | polypeptide 4 |
| 164 | Zm00001d0 | 0.5 | | protein; Expressed | AT5G16110 | 6.5 | | hypothetical protein |
| | 41410 | | | protein | | | | |
| 165 | Zm00001d0 | 0.5 | see2b | senescence | AT4G32940 | 1.7 | | GAMMAVPE |
| | 44495 | | | enhanced2b | | | | |
| 166 | Zm00001d0 | 0.5 | | Chaperone | AT1G16680 | 5.6 | | Chaperone DnaJ-domain |
| | 41488 | | | DnaJ-domain | | | | superfamily protein |
| | | | | superfamily protein | | | | |
| 167 | Zm00001d0 | 0.4 | | Serine | AT4G12910 | 6.9 | scpl20 | serine carboxypeptidase-like |
| | 41769 | | | carboxypeptidase- | | | | 20 |
| | | | | like20 | | | | |
| 168 | Zm00001d0 | 0.4 | | FAD/NAD(P)- | AT4G38540 | 0.4 | | FAD/NAD(P)-binding |
| | 48416 | | | binding | | | | oxidoreductase family |
| | | | | oxidoreductase family | | | | protein |
| | | | | protein | | | | |
| 169 | Zm00001d0 | 0.4 | | chaperone protein | AT2G24395 | 0.7 | | chaperone protein dnaJ-like |
| | 31514 | | | dnaJ-related | | | | protein |
| 170 | Zm00001d0 | 0.4 | | Ultraviolet-B- | AT2G06520 | 0.7 | PSBX | photosystem II subunit X |
| | 22464 | | | repressible | | | | |
| | | | | protein | | | | |
| 171 | Zm00001d0 | 0.4 | | Photosystem II repair | AT1G03600 | 2.6 | PSB27 | photosystem II family protein |
| | 29049 | | | protein PSB27-H1 | | | | |
| | | | | chloroplastic | | | | |
| 172 | Zm00001d0 | 0.4 | | | AT4G11910 | 3.6 | NYE2, | STAY-GREEN-like protein |
| | 21288 | | | | | | NONY | |
| | | | | | | | ELLOWING | |
| | | | | Senescence-inducible | | | 2, SGR2, | |
| | | | | chloroplast | | | STAY- | |
| | | | | stay-green | | | GREEN 2 | |
| | | | | protein 1 | AT4G22920 | 0.6 | NYE1 | ATNYE1, NON-YELLOWING |
| | | | | | | | | 1, SGR1, STAY-GREEN |
| | | | | | | | | 1, SGR, STAY-GREEN |
| 173 | Zm00001d0 | 0.4 | | RNase L inhibitor | AT5G10070 | 30.6 | | RNase L inhibitor protein-like |
| | 48190 | | | protein-related | | | | protein |
| 174 | Zm00001d0 | 0.4 | | GDSL | AT1G28580 | 0.3 | | GDSL-like |
| | 44465 | | | esterase/lipase | | | | Lipase/Acylhydrolase |
| | | | | | | | | superfamily protein |
| | | | | | AT1G28570 | 13.3 | | SGNH hydrolase-type |
| | | | | | | | | esterase superfamily protein |
| 175 | Zm00001d0 | 0.4 | rte2 | rotten ear2 | AT3G62270 | 2.1 | BOR2, | HCO3-transporter family |
| | 41590 | | | | | | REQUIRES | |
| | | | | | | | HIGH | |
| | | | | | | | BORON 2 | |
| 176 | Zm00001d0 | 0.4 | gst19 | glutathione | AT1G17170 | 0.8 | GSTU24 | ATGSTU24, glutathione S- |
| | 36951 | | | transferase19 | | | | transferase TAU |
| | | | | | | | | 24, GST, Arabidopsis thaliana |
| | | | | | | | | Glutathione S-transferase |
| | | | | | | | | (class tau) 24 |
| 177 | Zm00001d0 | 0.3 | amt1 | ammonium | AT4G13510 | 0.4 | AMT1; 1 | ATAMT1; 1, ATAMT1, |
| | 25831 | | | transporter1 | | | | ARABIDOPSIS THALIANA |
| | | | | | | | | AMMONIUM TRANSPORT 1 |
| 178 | Zm00001d0 | 0.3 | mdh4 | malate | AT1G04410 | 6.9 | c-NAD- | Lactate/malate |
| | 32695 | | | dehydrogenase4 | | | MDH1 | dehydrogenase family |
| | | | | | | | | protein |
| 179 | Zm00001d0 | 0.3 | | Cytochrome c | AT1G53030 | 11.8 | COX17 | Cytochrome C oxidase |
| | 52040 | | | oxidase copper | | | | copper chaperone (COX17) |
| | | | | chaperone%3B | | | | |
| | | | | Cytochrome c oxidase | | | | |
| | | | | copper chaperone | | | | |
| | | | | isoform 1% 3B | | | | |
| | | | | Cytochrome c oxidase | | | | |
| | | | | copper chaperone | | | | |
| | | | | isoform 2 | | | | |
| 180 | Zm00001d0 | 0.3 | | ATPase%2C | AT1G71960 | 3.6 | ABCG25 | ATABCG25, Arabidopsis |
| | 53049 | | | coupled | | | | thaliana ATP-binding |
| | | | | to transmembrane | | | | cassette G25 |
| | | | | movement of | | | | |
| | | | | substance%3B | | | | |
| | | | | ATPase%2C coupled | | | | |
| | | | | to transmembrane | | | | |
| | | | | movement of | | | | |
| | | | | substances | | | | |
| 181 | Zm00001d0 | 0.3 | | SGF29 tudor-like | AT3G27460 | 16.0 | SGF29a | AtSGF29a |
| | 23689 | | | domain | | | | |
| 182 | Zm00001d0 | 0.3 | | Transmembrane 9 | AT5G25100 | 0.1 | | Endomembrane protein 70 |
| | 24141 | | | superfamily | | | | protein family |
| | | | | member 9 | AT5G10840 | 1.3 | EMP1 | Endomembrane protein 70 |
| | | | | | | | | protein family |
| | | | | | AT2G24170 | 0.5 | | Endomembrane protein 70 |
| | | | | | | | | protein family |
| 183 | Zm00001d0 | 0.3 | | Serine | AT3G17180 | 0.5 | scpl33 | serine carboxypeptidase-like |
| | 40741 | | | carboxypeptidase- like 33 | | | | 33 |
| 184 | Zm00001d0 | 0.3 | | Protein FREE1 | AT1G20110 | 0.5 | FREE1 | RING/FYVE/PHD zinc finger |
| | 21878 | | | | | | | superfamily protein |
| 185 | Zm00001d0 | 0.3 | cys2 | cysteine synthase2 | AT4G14880 | 1.2 | OASA1 | ATCYS- |
| | 31136 | | | | | | | 3A, CYTACS1, OLD3, ONSET |
| | | | | | | | | OF LEAF DEATH 3 |
| 186 | Zm00001d0 | 0.3 | mate6 | multidrug and toxic | AT4G39030 | 0.3 | EDS5 | SCORD3, susceptible to |
| | 15060 | | | compound | | | | coronatine-deficient Pst |
| | | | | extrusion6 | | | | DC3000 3, SID1, SALICYLIC |
| | | | | | | | | ACID INDUCTION |
| | | | | | | | | DEFICIENT 1 |
| 187 | Zm00001d0 | 0.3 | | evolutionarily | AT1G79270 | 0.4 | ECT8 | evolutionarily conserved C- |
| | 43860 | | | conserved | | | | terminal region 8 |
| | | | | C-terminal region 8 | | | | |
| 188 | Zm00001d0 | 0.3 | | Photosystem II repair | AT1G03600 | 2.6 | PSB27 | photosystem II family protein |
| | 47532 | | | protein PSB27-H1 | | | | |
| | | | | chloroplastic | | | | |
| 189 | Zm00001d0 | 0.3 | | NAD(P)H | AT4G27270 | 1.1 | | Quinone reductase family |
| | 43249 | | | dehydrogenase | | | | protein |
| | | | | (quinone) FQR1 | | | | |
| 190 | Zm00001d0 | 0.3 | | Cytochrome | AT2G40890 | 3.4 | CYP98A3 | REF8, REDUCED EPIDERMAL |
| | 43174 | | | P450 98A3 | | | | FLUORESCENCE 8 |
| 191 | Zm00001d0 | 0.2 | | Tryptophan | AT1G34060 | 19.5 | | Pyridoxal phosphate (PLP)- |
| | 43651 | | | aminotransferase- | | | | dependent transferases |
| | | | | related protein 4 | | | | superfamily protein |
| 192 | Zm00001d0 | 0.2 | | ROTUNDIFOLIA | AT2G39705 | 0.6 | RTFL8 | DVL11, DEVIL 11 |
| | 47820 | | | like 8 | | | | |
| 193 | Zm00001d0 | 0.2 | | Phosphatidylinositol | AT4G00440 | 4.5 | TRM15 | GPI-anchored adhesin-like |
| | 48540 | | | N-acety- | | | | protein, putative (DUF3741) |
| | | | | glucosaminly- | | | | |
| | | | | transferase subunit P-related | | | | |
| 194 | Zm00001d0 | 0.2 | cyp11 | cytochrome P450 11 | AT3G14690 | 0.7 | CYP72A15 | cytochrome P450, family 72, |
| | 44159 | | | | | | | subfamily A, polypeptide 15 |
| 195 | Zm00001d0 | 0.2 | | F11F12.5 protein | AT3G20300 | 4.4 | | extracellular ligand-gated ion |
| | 46652 | | | | | | | channel protein (DUF3537) |
| 196 | Zm00001d0 | 0.2 | | Photosystem II | AT2G30570 | 0.4 | PSBW | photosystem II reaction |
| | 43299 | | | reaction | | | | center W |
| | | | | center W protein | | | | |
| | | | | chloroplastic | | | | |
| 197 | Zm00001d0 | 0.2 | | Ribosomal protein | AT4G22380 | 0.3 | | Ribosomal protein |
| | 47958 | | | L7Ae/L30e/S12e/ | | | | L7Ae/L30e/S12e/Gadd45 |
| | | | | Gadd4 | | | | family protein |
| | | | | 5 family protein | | | | |
| 198 | Zm00001d0 | 0.2 | | Grx_A2-gluta | AT4G33040 | 0.8 | | Thioredoxin superfamily |
| | 39468 | | | redoxin | | | | protein |
| | | | | subgroup III | | | | |
| 199 | Zm00001d0 | 0.2 | | Putative membrane | AT5G59350 | 8.9 | | transmembrane protein |
| | 37644 | | | lipoprotein | | | | |
| 200 | Zm00001d0 | 0.2 | | HIT-type Zinc finger | AT4G28820 | 14.1 | | HIT-type Zinc finger family |
| | 42997 | | | family protein | | | | protein |
| 201 | Zm00001d0 | 0.2 | | PIF/Ping-Pong | AT5G12010 | 0.5 | | nuclease |
| | 44300 | | | family | | | | |
| | | | | of plant transposases | | | | |
| 202 | Zm00001d0 | 0.2 | | Glutathione S- | AT1G59700 | 0.4 | GSTU16 | ATGSTU16, glutathione S- |
| | 43795 | | | transferase GSTU6 | | | | transferase TAU 16 |
| | | | | | AT1G59670 | 0.6 | GSTU15 | ATGSTU15, glutathione S- |
| | | | | | | | | transferase TAU 15 |
| 203 | Zm00001d0 | 0.1 | | GINS complex | AT1G19080 | 10.2 | TTN10 | PSF3, Partner of SLD5 3 |
| | 52742 | | | protein | | | | |
| 204 | Zm00001d0 | 0.1 | | Photosystem II core | AT1G67740 | 4.9 | PSBY | YCF32 |
| | 49650 | | | complex protein psbY | | | | |
| 205 | Zm00001d0 | 0.1 | | 5-hydroxyisourate | AT5G58220 | 0.3 | TTL | ALNS, allantoin synthase |
| | 47217 | | | hydrolase | | | | |
| 206 | Zm00001d0 | 0.1 | | Tryptophan | AT1G34060 | 19.5 | | Pyridoxal phosphate (PLP)- |
| | 43650 | | | aminotransferase- | | | | dependent transferases |
| | | | | related protein 4 | | | | superfamily protein |
| 207 | Zm00001d0 | 0.1 | | F-box/kelch-repeat | AT2G44130 | 1.2 | KFB39, | Galactose oxidase/kelch |
| | 49016 | | | protein SKIP20 | | | KMD3, | repeat superfamily protein, |
| | | | | | | | KISS | Kelch-domain-containing F- |
| | | | | | | | ME | box protein 39 |
| | | | | | | | DEADLY | |
| | | | | | | | 3 | |
| 208 | Zm00001d0 | 0.1 | gid1 | gibberellin- | AT3G05120 | 1.4 | GID1A | ATGIDIA, GA INSENSITIVE |
| | 38165 | | | insensitive | | | | DWARF1A |
| | | | | dwarf protein | | | | |
| | | | | homolog1 | AT3G63010 | 0.5 | GID1B | ATGID1B |
| 209 | Zm00001d0 | 0.0 | | cytoplasmic | AT1G33490 | 0.7 | | E3 ubiquitin-protein ligase |
| | 53786 | | | membrane | | | | |
| | | | | protein | | | | |

TABLE 7
224 MAIZE NON-TRANSCRIPTION FACTORS

| | | | | Machine |
|---|---|---|---|---|
| | | | | learning Gene |
| | | | | Importance to |
| | | | | NUE |
| | | | | (Cheng & |
| | | | | Coruzzi 2021, |
| Row | Gene | Symbol | Description | Table S3) |
| 1 | Zm00001d002530 | | | 169.7 |
| 2 | Zm00001d002426 | morf2 | multiple organellar RNA editing | 128.9 |
| | | | factor2 | |
| 3 | Zm00001d001857 | | | 96.5 |
| 4 | Zm00001d002854 | | | 90.8 |
| 5 | Zm00001d001804 | mlo9 | barley mio defense gene | 74.4 |
| | | | homolog9 | |
| 6 | Zm00001d002880 | imd2 | isopropylmalate dehydrogenase2 | 71.4 |
| 7 | Zm00001d003767 | pco139896 | Photosystem I subunit O | 59.7 |
| 8 | Zm00001d003059 | | Probable low-specificity | 51.0 |
| | | | L-threonine aldolase 1 | |
| 9 | Zm00001d002261 | | Peroxisomal (S)-2-hydroxy-acid | 40.8 |
| | | | oxidase GLO1 | |
| 10 | Zm00001d002798 | | OJ000126_13.10 protein; protein | 39.3 |
| 11 | Zm00001d002503 | | ABC transporter C family member 9 | 38.5 |
| 12 | Zm00001d005446 | | Photosystem I reaction center | 36.7 |
| | | | subunit IV A | |
| 13 | Zm00001d002826 | | SAUR11-auxin-responsive SAUR | 30.3 |
| | | | family member | |
| 14 | Zm00001d006098 | | Cyclopropane fatty acid synthase | 26.9 |
| 15 | Zm00001d008379 | cys1 | cysteine synthasel | 26.1 |
| 16 | Zm00001d003941 | | Probable metal-nicotianamine | 20.8 |
| | | | transporter YSL6 | |
| 17 | Zm00001d003129 | hct5 | hydroxycinnamoyltransferase5 | 18.6 |
| 18 | Zm00001d003457 | | PLAT domain-containing protein 3 | 17.5 |
| 19 | Zm00001d005317 | pco080190 | Amino acid binding protein | 17.1 |
| 20 | Zm00001d006793 | | Cysteine-rich receptor-like | 16.1 |
| | | | protein kinase 10 | |
| 21 | Zm00001d002999 | ga2ox2 | gibberellin 2-oxidase2 | 15.3 |
| 22 | Zm00001d007827 | elip1 | early light inducible proteinl | 15.0 |
| 23 | Zm00001d005996 | | Photosystem I reaction center | 14.6 |
| | | | subunit V | |
| 24 | Zm00001d007857 | pspb1 | photosystem II oxygen evolving | 13.4 |
| | | | polypeptide1 | |
| 25 | Zm00001d006267 | | Serine/threonine-protein kinase | 12.6 |
| | | | STY46 | |
| 26 | Zm00001d006193 | | cytochrome P450 family 78 | 12.5 |
| | | | subfamily A polypeptide 8 | |
| 27 | Zm00001d005193 | | Protein LURP1 | 12.3 |
| 28 | Zm00001d003446 | IDP2449 | Gamma-glutamyltranspeptidase 1 | 12.0 |
| 29 | Zm00001d007383 | | Probable alpha-mannosidase | 11.7 |
| 30 | Zm00001d003459 | cl11315_1a | Protein disulfide-isomerase LQY1 | 8.6 |
| | | | chloroplastic | |
| 31 | Zm00001d007274 | | Protein kinase Kelch repeat: Kelch | 8.1 |
| 32 | Zm00001d006140 | | UDP-glycosyltransferase 74B1 | 7.5 |
| 33 | Zm00001d029853 | pza03240 | Proline oxidase | 7.1 |
| 34 | Zm00001d010867 | pco112665 | Bifunctional protein FolD 2 | 5.9 |
| 35 | Zm00001d005657 | | | 5.9 |
| 36 | Zm00001d020348 | | | 5.7 |
| 37 | Zm00001d006540 | | Oxygen-evolving enhancer | 5.7 |
| | | | protein 3-1 | |
| 38 | Zm00001d011188 | | 3′-5′ exonuclease domain- | 5.7 |
| | | | containing protein/K homology | |
| | | | domain-containing protein/KH | |
| | | | domain-containing protein | |
| 39 | Zm00001d008974 | | Mitochondrial arginine | 5.4 |
| | | | transporter BAC2 | |
| 40 | Zm00001d006663 | lhca1 | light harvesting complex A1 | 5.4 |
| 41 | Zm00001d008772 | | Serinc-domain containing serine | 4.7 |
| | | | and sphingolipid biosynthesis | |
| | | | protein | |
| 42 | Zm00001d017768 | | | 4.2 |
| 43 | Zm00001d024637 | | L-type lectin-domain containing | 4.2 |
| | | | receptor kinase V.9 | |
| 44 | Zm00001d025665 | umc1272 | Probable amino acid permease 7 | 4.1 |
| 45 | Zm00001d017186 | hct6 | hydroxycinnamoyltransferase6 | 4.0 |
| 46 | Zm00001d014961 | | Tetratricopeptide repeat (TPR)- | 3.9 |
| | | | like superfamily protein | |
| 47 | Zm00001d013086 | AY109733 | 40S ribosomal protein S18 | 3.7 |
| 48 | Zm00001d006137 | | UDP-glycosyltransferase 74B1 | 3.7 |
| 49 | Zm00001d005997 | mlkp3 | Maize LINC KASH AtWIP-like3 | 3.6 |
| 50 | Zm00001d026599 | lhcb6 | light harvesting chlorophyll a/b | 3.5 |
| | | | binding protein6 | |
| 51 | Zm00001d006274 | TIDP2961 | Auxin-responsive protein SAUR61 | 3.4 |
| 52 | Zm00001d021334 | | Thioredoxin-like protein CDSP32 | 3.4 |
| | | | chloroplastic | |
| 53 | Zm00001d006160 | | DEAD-box ATP-dependent RNA | 3.3 |
| | | | helicase 7 | |
| 54 | Zm00001d038088 | | Sm-like protein LSM5 | 3.0 |
| 55 | Zm00001d008681 | GRMZM2G380414 | Ultraviolet-B-repressible protein | 3.0 |
| 56 | Zm00001d018468 | fdh1 | formaldehyde dehydrogenase | 2.9 |
| | | | homolog1 | |
| 57 | Zm00001d006644 | mkkk27 | MAP kinase kinase kinase27 | 2.9 |
| 58 | Zm00001d011285 | lhcb10 | light harvesting chlorophyll a/b | 2.9 |
| | | | binding protein10 | |
| 59 | Zm00001d009178 | | Serine carboxypeptidase-like 33 | 2.9 |
| 60 | Zm00001d031677 | pco070301 | MtN19-like protein | 2.8 |
| 61 | Zm00001d016100 | | Rhodanese-like domain- | 2.8 |
| | | | containing protein 4 chloroplastic | |
| 62 | Zm00001d006521 | | | 2.7 |
| 63 | Zm00001d010463 | | Phospholipase A1-lgamma1 | 2.6 |
| | | | chloroplastic | |
| 64 | Zm00001d045054 | stc1 | sesquiterpene cyclase1 | 2.5 |
| 65 | Zm00001d011679 | nrt5 | nitrate transports | 2.5 |
| 66 | Zm00001d032605 | npi447a | agal1; alpha-galactosidase1: | 2.5 |
| | | | Entrez Gene relates to alpha- | |
| | | | galactosidase 1 (AGAL) of | |
| | | | Arabidopsis | |
| 67 | Zm00001d036535 | oec33 | oxygen evolving complex, 33 kDa | 2.5 |
| | | | subunit | |
| 68 | Zm00001d011642 | pco129777b | Phosphoethanolamine N- | 2.4 |
| | | | methyltransferase 3 | |
| 69 | Zm00001d011418 | | cytochrome P450 family 72 | 2.4 |
| | | | subfamily A polypeptide 8 | |
| 70 | Zm00001d025644 | | Agmatine deiminase | 2.4 |
| 71 | Zm00001d025915 | | Syntaxin-81 | 2.4 |
| 72 | Zm00001d019899 | | Rhodanese-like domain- | 2.4 |
| | | | containing protein 9 chloroplastic | |
| 73 | Zm00001d014303 | | Vacuolar-sorting receptor 4 | 2.3 |
| 74 | Zm00001d025103 | amo1 | amine oxidase1 | 2.3 |
| 75 | Zm00001d013146 | | Photosystem I reaction center | 2.3 |
| | | | subunit III chloroplastic | |
| 76 | Zm00001d038984 | psah1 | photosystem I H subunit1 | 2.3 |
| 77 | Zm00001d008691 | atgl8d | autophagy18d | 2.3 |
| 78 | Zm00001d026026 | | | 2.3 |
| 79 | Zm00001d013493 | lox5 | lipoxygenases | 2.1 |
| 80 | Zm00001d027373 | cdc2 | cell division control protein | 2.1 |
| | | | homolog2 | |
| 81 | Zm00001d042541 | lox1 | lipoxygenase1 | 2.1 |
| 82 | Zm00001d021763 | psb29 | photosystem II subunit29 | 2.1 |
| 83 | Zm00001d010796 | hex3 | hexokinase3 | 2.1 |
| 84 | Zm00001d034796 | | Vacuolar protein sorting- | 2.1 |
| | | | associated protein 9A | |
| 85 | Zm00001d021021 | | Peptidyl-prolyl cis-trans | 2.0 |
| | | | isomerase | |
| 86 | Zm00001d009084 | | S-adenosyl-L-methionine- | 2.0 |
| | | | dependent methyltransferases | |
| | | | superfamily protein | |
| 87 | Zm00001d011624 | | alpha/beta-Hydrolases | 1.9 |
| | | | superfamily protein | |
| 88 | Zm00001d038891 | peamt2 | Phosphoethanolamine | 1.9 |
| | | | N-methyltransferase 3 | |
| 89 | Zm00001d024306 | | Methyl-CpG-binding domain- | 1.8 |
| | | | containing protein 13 | |
| 90 | Zm00001d016561 | | chaperone protein dnaJ-related | 1.8 |
| 91 | Zm00001d009967 | sqd1 | sulfolipid biosynthesis1 | 1.8 |
| 92 | Zm00001d030766 | | | 1.8 |
| 93 | Zm00001d024568 | mpk4 | MAP kinase4 | 1.8 |
| 94 | Zm00001d006900 | | Phospho-2-dehydro-3- | 1.7 |
| | | | deoxyheptonate aldolase 2 | |
| | | | chloroplastic | |
| 95 | Zm00001d037695 | | actin binding protein family | 1.7 |
| 96 | Zm00001d032306 | | | 1.7 |
| 97 | Zm00001d039118 | rl1 | radialis homolog1 | 1.7 |
| 98 | Zm00001d010791 | | Histidine-containing | 1.5 |
| | | | phosphotransfer protein 4 | |
| 99 | Zm00001d006211 | | Protein STAY-GREEN 1 | 1.5 |
| | | | chloroplastic | |
| 100 | Zm00001d048497 | | | 1.5 |
| 101 | Zm00001d015058 | | Histone deacetylase complex | 1.5 |
| | | | subunit SAP18 | |
| 102 | Zm00001d034832 | | Probable acyl-activating enzyme | 1.5 |
| | | | 16 chloroplastic | |
| 103 | Zm00001d050403 | | Chlorophyll a-b binding protein 4 | 1.5 |
| 104 | Zm00001d031447 | mrpa10 | multidrug resistance protein | 1.4 |
| | | | associated10 | |
| 105 | Zm00001d016800 | | | 1.4 |
| 106 | Zm00001d020877 | | Photosystem I reaction center | 1.4 |
| | | | subunit V chloroplastic | |
| 107 | Zm00001d043757 | | Nuclear pore complex protein | 1.3 |
| | | | NUP50A | |
| 108 | Zm00001d019518 | gpm930 | Photosystem I reaction center | 1.3 |
| | | | subunit IV A | |
| 109 | Zm00001d027444 | IDP755 | D111/G-patch domain-containing | 1.3 |
| | | | protein | |
| 110 | Zm00001d048944 | | GTP-binding protein hfIX | 1.3 |
| 111 | Zm00001d053696 | | Glucomannan 4-beta- | 1.3 |
| | | | mannosyltransferase 2 | |
| 112 | Zm00001d033132 | umc1383 | lhcb9; light harvesting chlorophyll | 1.3 |
| | | | binding protein9: cDNA sequence | |
| | | | is a classII lhcb, unlike previously | |
| | | | characterized lhcb genes which | |
| | | | are class1 (Viret et al 1993) | |
| 113 | Zm00001d021435 | lhcb2 | light harvesting chlorophyll a/b | 1.2 |
| | | | binding protein2 | |
| 114 | Zm00001d011799 | | Nuclear transport factor 2 (NTF2) | 1.2 |
| | | | family protein | |
| 115 | Zm00001d052518 | | Pollen Ole e 1 allergen and | 1.2 |
| | | | extensin family protein | |
| 116 | Zm00001d021906 | | Chlorophyll a-b binding protein | 1.1 |
| 117 | Zm00001d020264 | | | 1.1 |
| 118 | Zm00001d017526 | pip1b | plasma membrane intrinsic | 1.1 |
| | | | proteinl | |
| 119 | Zm00001d016991 | | | 1.1 |
| 120 | Zm00001d014435 | | D-xylose-proton symporter-like l | 1.1 |
| 121 | Zm00001d013039 | psad1 | photosystem I subunit d1 | 1.1 |
| 122 | Zm00001d044401 | | photosystem II light harvesting | 1.1 |
| | | | complex gene B1B2 | |
| 123 | Zm00001d022181 | | Phospho-2-dehydro-3- | 1.1 |
| | | | deoxyheptonate aldolase 1 | |
| 124 | Zm00001d032042 | AY111834 | Cytochrome P450 CYP78A53 | 1.1 |
| 125 | Zm00001d018940 | elip2 | early light inducible protein2 | 1.1 |
| 126 | Zm00001d032197 | | Chlorophyll a-b binding protein 4 | 1.1 |
| | | | chloroplastic | |
| 127 | Zm00001d013465 | d9 | dwarf plant9 | 1.1 |
| 128 | Zm00001d040190 | | hydroxyproline-rich glycoprotein | 1.1 |
| | | | family protein | |
| 129 | Zm00001d034422 | | 40S ribosomal protein S18 | 1.1 |
| 130 | Zm00001d043248 | | Transcription factor bHLH112 | 1.0 |
| 131 | Zm00001d026635 | alla1 | allantoinase1 | 1.0 |
| 132 | Zm00001d032152 | cncr1 | cinnamoyl CoA reductasel | 1.0 |
| 133 | Zm00001d052837 | | BTB/POZ domain-containing | 1.0 |
| | | | protein | |
| 134 | Zm00001d018779 | pspb2 | photosystem II oxygen evolving | 1.0 |
| | | | polypeptide2 | |
| 135 | Zm00001d027861 | | Alanine--glyoxylate | 1.0 |
| | | | aminotransferase 2 homolog l | |
| | | | mitochondrial | |
| 136 | Zm00001d012609 | | Serine/threonine-protein kinase | 0.9 |
| 137 | Zm00001d032233 | | Encodes a protein whose | 0.9 |
| | | | expression is responsive to | |
| | | | nematode infection. | |
| 138 | Zm00001d025035 | | Putative D-mannose binding | 0.9 |
| | | | lectin family receptor-like protein | |
| | | | kinase | |
| 139 | Zm00001d050551 | | ubiquitin-associated (UBA)/TS-N | 0.9 |
| | | | domain-containing protein | |
| 140 | Zm00001d038346 | | | 0.9 |
| 141 | Zm00001d019117 | | | 0.9 |
| 142 | Zm00001d043374 | | Peptide transporter PTR2 | 0.8 |
| 143 | Zm00001d041819 | psan1 | photosystem I N subunitl | 0.8 |
| 144 | Zm00001d027456 | pba1 | PBA1 homolog1 | 0.8 |
| 145 | Zm00001d023713 | psan2 | photosystem I N subunit2 | 0.8 |
| 146 | Zm00001d027601 | TIDP3460 | cytochrome P450 family 96 | 0.8 |
| | | | subfamily A polypeptide 1 | |
| 147 | Zm00001d051842 | | Zn-dependent hydrolase % 2C | 0.8 |
| | | | including glyoxylase | |
| 148 | Zm00001d046682 | | Peroxiredoxin-5 | 0.8 |
| 149 | Zm00001d046438 | ago101 | argonaute101 | 0.8 |
| 150 | Zm00001d022182 | | alpha/beta-Hydrolases | 0.8 |
| | | | superfamily protein | |
| 151 | Zm00001d017379 | | Thioredoxin M1 chloroplastic | 0.8 |
| 152 | Zm00001d016825 | | SPIa/RYanodine receptor (SPRY) | 0.8 |
| | | | domain-containing protein | |
| 153 | Zm00001d043621 | | Phosphatase phospho1 | 0.7 |
| 154 | Zm00001d047797 | | UDP-glucuronic acid | 0.7 |
| | | | decarboxylase 5 | |
| 155 | Zm00001d033419 | | Probable BOI-related E3 | 0.7 |
| | | | ubiquitin-protein ligase 2 | |
| 156 | Zm00001d044396 | IDP518 | Chlorophyll a-b binding protein | 0.7 |
| | | | 48% 2C chloroplastic | |
| 157 | Zm00001d027557 | gst31 | glutathione transferase31 | 0.7 |
| 158 | Zm00001d036274 | pco123453 | S-adenosyl-L-methionine- | 0.7 |
| | | | dependent methyltransferases | |
| | | | superfamily protein | |
| 159 | Zm00001d011487 | idh1 | isocitrate dehydrogenasel | 0.6 |
| 160 | Zm00001d051872 | pip1e | plasma membrane intrinsic | 0.6 |
| | | | protein1 | |
| 161 | Zm00001d009029 | | Putative leucine-rich repeat | 0.6 |
| | | | receptor-like protein kinase | |
| | | | family protein | |
| 162 | Zm00001d039914 | | Metallothionein-like protein type 2 | 0.6 |
| 163 | Zm00001d018364 | | Snfl-related kinase interacting | 0.6 |
| | | | protein SKI1 | |
| 164 | Zm00001d028598 | | ROTUNDIFOLIA like 8 | 0.6 |
| 165 | Zm00001d025892 | | ATPase | 0.6 |
| 166 | Zm00001d039715 | | Ultraviolet-B-repressible protein | 0.6 |
| 167 | Zm00001d029065 | | Protein LRP16 | 0.6 |
| 168 | Zm00001d047124 | | Proline oxidase | 0.6 |
| 169 | Zm00001d028360 | | NAD(P)-linked oxidoreductase | 0.6 |
| | | | superfamily protein | |
| 170 | Zm00001d034420 | gdh1 | glutamic dehydrogenasel | 0.6 |
| 171 | Zm00001d023560 | | Putative calcium-dependent | 0.6 |
| | | | protein kinase family protein | |
| 172 | Zm00001d012607 | gpm345 | NAD(P)H dehydrogenase | 0.5 |
| | | | (quinone) FQR1 | |
| 173 | Zm00001d014564 | oec33b | oxygen-evolving complex 33 kda | 0.5 |
| | | | protein b | |
| 174 | Zm00001d044056 | kch1 | potassium channel 1 | 0.5 |
| 175 | Zm00001d044243 | | 3′-5′ exonuclease domain- | 0.5 |
| | | | containing protein/K homology | |
| | | | domain-containing protein/KH | |
| | | | domain-containing protein | |
| 176 | Zm00001d050021 | abh3 | abscisic acid 8′-hydroxylase3 | 0.5 |
| 177 | Zm00001d041410 | | protein; Expressed protein | 0.5 |
| 178 | Zm00001d044495 | see2b | senescence enhanced2b | 0.5 |
| 179 | Zm00001d041488 | | Chaperone DnaJ-domain | 0.5 |
| | | | superfamily protein | |
| 180 | Zm00001d041769 | | Serine carboxypeptidase-like 20 | 0.4 |
| 181 | Zm00001d048416 | | FAD/NAD(P)-binding | 0.4 |
| | | | oxidoreductase family protein | |
| 182 | Zm00001d031514 | | chaperone protein dnaJ-related | 0.4 |
| 183 | Zm00001d022464 | | Ultraviolet-B-repressible protein | 0.4 |
| 184 | Zm00001d029049 | | Photosystem II repair protein | 0.4 |
| | | | PSB27-H1 chloroplastic | |
| 185 | Zm00001d021288 | | Senescence-inducible chloroplast | 0.4 |
| | | | stay-green protein 1 | |
| 186 | Zm00001d048190 | | RNase L inhibitor protein-related | 0.4 |
| 187 | Zm00001d044465 | | GDSL esterase/lipase | 0.4 |
| 188 | Zm00001d041590 | rte2 | rotten ear2 | 0.4 |
| 189 | Zm00001d036951 | gst19 | glutathione transferase19 | 0.4 |
| 190 | Zm00001d025831 | amt1 | ammonium transporter1 | 0.3 |
| 191 | Zm00001d032695 | mdh4 | malate dehydrogenase4 | 0.3 |
| 192 | Zm00001d052040 | | Cytochrome c oxidase copper | 0.3 |
| | | | chaperone % 3B Cytochrome c | |
| | | | oxidase copper chaperone | |
| | | | isoform 1% 3B Cytochrome c | |
| | | | oxidase copper chaperone | |
| | | | isoform 2 | |
| 193 | Zm00001d053049 | | ATPase % 2C coupled to | 0.3 |
| | | | transmembrane movement of | |
| | | | substance % 3B ATPase % 2C | |
| | | | coupled to transmembrane | |
| | | | movement of substances | |
| 194 | Zm00001d023689 | | SGF29 tudor-like domain | 0.3 |
| 195 | Zm00001d024141 | | Transmembrane 9 superfamily | 0.3 |
| | | | member 9 | |
| 196 | Zm00001d040741 | | Serine carboxypeptidase-like 33 | 0.3 |
| 197 | Zm00001d021878 | | Protein FREE1 | 0.3 |
| 198 | Zm00001d031136 | cys2 | cysteine synthase2 | 0.3 |
| 199 | Zm00001d015060 | mate6 | multidrug and toxic compound | 0.3 |
| | | | extrusion6 | |
| 200 | Zm00001d043860 | | evolutionarily conserved | 0.3 |
| | | | C-terminal region 8 | |
| 201 | Zm00001d047532 | | Photosystem II repair protein | 0.3 |
| | | | PSB27-H1 chloroplastic | |
| 202 | Zm00001d043249 | | NAD(P)H dehydrogenase | 0.3 |
| | | | (quinone) FQR1 | |
| 203 | Zm00001d043174 | GRMZM2G138074 | Cytochrome P450 98A3 | 0.3 |
| 204 | Zm00001d043651 | | Tryptophan aminotransferase- | 0.2 |
| | | | related protein 4 | |
| 205 | Zm00001d047820 | | ROTUNDIFOLIA like 8 | 0.2 |
| 206 | Zm00001d048540 | | Phosphatidylinositol N- | 0.2 |
| | | | acetyglucosaminlytransferase | |
| | | | subunit P-related | |
| 207 | Zm00001d048135 | | | 0.2 |
| 208 | Zm00001d044159 | cyp11 | cytochrome P450 11 | 0.2 |
| 209 | Zm00001d046652 | | F11F12.5 protein | 0.2 |
| 210 | Zm00001d043299 | | Photosystem II reaction center W | 0.2 |
| | | | protein chloroplastic | |
| 211 | Zm00001d047958 | | Ribosomal protein | 0.2 |
| | | | L7Ae/L30e/S12e/Gadd45 family | |
| | | | protein | |
| 212 | Zm00001d039468 | | Grx_A2-glutaredoxin subgroup III | 0.2 |
| 213 | Zm00001d037644 | | Putative membrane lipoprotein | 0.2 |
| 214 | Zm00001d042997 | | HIT-type Zinc finger family | 0.2 |
| | | | protein | |
| 215 | Zm00001d044300 | | PIF/Ping-Pong family of plant | 0.2 |
| | | | transposases | |
| 216 | Zm00001d043795 | | Glutathione S-transferase GSTU6 | 0.2 |
| 217 | Zm00001d038964 | | | 0.2 |
| 218 | Zm00001d052742 | | GINS complex protein | 0.1 |
| 219 | Zm00001d049650 | | Photosystem II core complex | 0.1 |
| | | | protein psbY | |
| 220 | Zm00001d047217 | | 5-hydroxyisourate hydrolase | 0.1 |
| 221 | Zm00001d043650 | | Tryptophan aminotransferase- | 0.1 |
| | | | related protein 4 | |
| 222 | Zm00001d049016 | | F-box/kelch-repeat protein | 0.1 |
| | | | SKIP20 | |
| 223 | Zm00001d038165 | gid1 | gibberellin-insensitive dwarf | 0.1 |
| | | | protein homolog1 | |
| 224 | Zm00001d053786 | | cytoplasmic membrane protein | 0.0 |

TABLE 8
547 ARABIDOPSIS NON-TRANSCRIPTION FACTORS

| | | | | Machine |
|---|---|---|---|---|
| | | | | learning |
| | | | | Gene |
| | | | | Importance |
| | | | | to NUE |
| | | | | (Cheng & |
| | | | | Coruzzi |
| | | | | 2021, |
| Row | Gene | Symbol | Description | Table S3) |
| 1 | AT5G10070 | | RNase L inhibitor protein-like protein | 30.6 |
| 2 | AT1G20550 | | O-fucosyltransferase family protein | 21.0 |
| 3 | AT3G59800 | | stress response protein | 20.4 |
| 4 | AT4G03240 | FH | ATFH | 20.3 |
| 5 | AT1G34060 | | Pyridoxal phosphate (PLP)-dependent transferases superfamily | 19.5 |
| | | | protein | |
| 6 | AT5G04940 | SUVH1 | SU(VAR)3-9 homolog 1 | 19.0 |
| 7 | AT1G08630 | THA1 | threonine aldolase 1 | 18.0 |
| 8 | AT4G22340 | CDS2 | cytidinediphosphate diacylglycerol synthase 2 | 17.6 |
| 9 | AT2G34200 | | RING/FYVE/PHD zinc finger superfamily protein | 17.6 |
| 10 | AT1G79390 | | centrosomal protein | 17.4 |
| 11 | AT3G10220 | EMB2804 | tubulin folding cofactor B | 16.6 |
| 12 | AT2G29960 | CYP5 | ATCYP5, ARABIDOPSIS THALIANA CYCLOPHILIN 5, CYP19-4, | 16.2 |
| | | | CYCLOPHILIN 19-4 | |
| 13 | AT3G27460 | SGF29a | AtSGF29a | 16.0 |
| 14 | AT4G04955 | ALN | ATALN,allantoinase | 15.7 |
| 15 | AT5G53920 | | ribosomal protein L11 methyltransferase-like protein | 15.3 |
| 16 | AT5G59210 | | myosin heavy chain-like protein | 15.3 |
| 17 | AT1G35470 | RanBPM | SPIa/RYanodine receptor (SPRY) domain-containing protein | 15.3 |
| 18 | AT4G39660 | AGT2 | alanine: glyoxylate aminotransferase 2 | 14.4 |
| 19 | AT4G28820 | | HIT-type Zinc finger family protein | 14.1 |
| 20 | AT5G56460 | | Protein kinase superfamily protein | 13.9 |
| 21 | AT1G28570 | | SGNH hydrolase-type esterase superfamily protein | 13.3 |
| 22 | AT5G46620 | | hypothetical protein | 13.3 |
| 23 | AT5G57000 | | DEAD-box ATP-dependent RNA helicase | 12.5 |
| 24 | AT1G63980 | | D111/G-patch domain-containing protein | 12.5 |
| 25 | AT1G53030 | | Cytochrome C oxidase copper chaperone (COX17) | 11.8 |
| 26 | AT5G65480 | | CCI1, Clavata complex interactor 1 | 11.7 |
| 27 | AT4G32660 | AME3 | Protein kinase superfamily protein | 11.6 |
| 28 | AT1G66480 | | plastid movement impaired 2 | 11.5 |
| 29 | AT3G13224 | | RNA-binding (RRM/RBD/RNP motifs) family protein | 11.0 |
| 30 | AT3G20650 | | mRNA capping enzyme family protein | 10.6 |
| 31 | AT1G55760 | SIBP1 | BTB/POZ domain-containing protein | 10.5 |
| 32 | AT1G22160 | | senescence-associated family protein (DUF581) | 10.4 |
| 33 | AT1G19080 | TTN10 | PSF3, Partner of SLD5 3 | 10.2 |
| 34 | AT4G33030 | SQD1 | sulfoquinovosyldiacylglycerol l | 9.8 |
| 35 | AT1G52080 | AR791 | actin binding protein family | 9.7 |
| 36 | AT4G01320 | ATSTE24 | Peptidase family M48 family protein | 9.6 |
| 37 | AT5G10100 | TPPI | Haloacid dehalogenase-like hydrolase (HAD) superfamily protein | 9.6 |
| 38 | AT3G01560 | | proline-rich receptor-like kinase, putative (DUF1421) | 9.3 |
| 39 | AT4G39955 | | alpha/beta-Hydrolases superfamily protein | 9.1 |
| 40 | AT4G21200 | GA20X8 | ATGA2OX8, ARABIDOPSIS THALIANA GIBBERELLIN 2-OXIDASE 8 | 9.1 |
| 41 | AT5G38220 | | alpha/beta-Hydrolases superfamily protein | 9.1 |
| 42 | AT5G64740 | CESA6 | E112, IXR2, ISOXABEN RESISTANT 2, PRC1, PROCUSTE 1 | 9.1 |
| 43 | AT5G08370 | AGAL2 | AtAGAL2, alpha-galactosidase 2 | 9.0 |
| 44 | AT1G14560 | CoAc1, CoA Carrier 1 | Mitochondrial substrate carrier family protein | 9.0 |
| 45 | AT5G59350 | | transmembrane protein | 8.9 |
| 46 | AT5G18130 | | transmembrane protein | 8.6 |
| 47 | AT5G64160 | | plant/protein | 8.4 |
| 48 | AT4G09800 | RPS18C | S18 ribosomal protein | 8.2 |
| 49 | AT4G11560 | | bromo-adjacent homology (BAH) domain-containing protein | 8.0 |
| 50 | AT1G12940 | NRT2.5 | ATNRT2.5, nitrate transporter2.5 | 7.9 |
| 51 | AT3G23790 | AAE16 | AMP-dependent synthetase and ligase family protein | 7.7 |
| 52 | AT4G25850 | ORP4B | OSBP(oxysterol binding protein)-related protein 4B | 7.5 |
| 53 | AT1G58080 | ATP-PRT1 | ATATP-PRT1, ATP phosphoribosyl transferase 1, HISN1A | 7.2 |
| 54 | AT1G15110 | PSS1 | AtPSS1 | 7.0 |
| 55 | AT1G04410 | c-NAD-MDHl | Lactate/malate dehydrogenase family protein | 6.9 |
| 56 | AT4G12910 | scpl20 | serine carboxypeptidase-like 20 | 6.9 |
| 57 | AT5G04090 | | histidine-tRNA ligase | 6.8 |
| 58 | AT1G03340 | | hypothetical protein | 6.8 |
| 59 | AT1G05560 | UGT75B1 | UGT1, UDP-GLUCOSE TRANSFERASE 1 | 6.8 |
| 60 | AT5G09300 | | Thiamin diphosphate-binding fold (THDP-binding) superfamily | 6.7 |
| | | | protein | |
| 61 | AT2G44950 | HUB1 | RDO4, REDUCED DORMANCY 4 | 6.5 |
| 62 | AT5G1611O | | hypothetical protein | 6.5 |
| 63 | AT3G13730 | CYP90D1 | cytochrome P450, family 90, subfamily D, polypeptide 1 | 6.4 |
| 64 | AT5G59050 | | G patch domain protein | 6.4 |
| 65 | AT4G38250 | | Transmembrane amino acid transporter family protein | 6.3 |
| 66 | AT2G45640 | SAP18 | ATSAP18, SIN3 ASSOCIATED POLYPEPTIDE 18 | 6.3 |
| 67 | AT1G73720 | SMU1 | transducin family protein/WD-40 repeat family protein | 6.3 |
| 68 | AT3G25220 | FKBP15-1 | FK506-binding protein 15 kD-1 | 6.2 |
| 69 | AT4G00330 | CRCK2 | calmodulin-binding receptor-like cytoplasmic kinase 2 | 6.2 |
| 70 | AT1G51560 | | Pyridoxamine 5′-phosphate oxidase family protein | 6.1 |
| 71 | AT5G19420 | | Regulator of chromosome condensation (RCC1) family with FYVE zinc | 6.1 |
| | | | finger domain-containing protein | |
| 72 | AT3G47010 | | Glycosyl hydrolase family protein | 6.0 |
| 73 | AT1G42430 | | inactive purple acid phosphatase-like protein | 5.8 |
| 74 | AT1G15710 | | prephenate dehydrogenase family protein | 5.6 |
| 75 | AT1G16680 | | Chaperone DnaJ-domain superfamily protein | 5.6 |
| 76 | AT4G33180 | | alpha/beta-Hydrolases superfamily protein | 5.6 |
| 77 | AT1G08730 | XIC | Myosin family protein with Dil domain-containing protein | 5.5 |
| 78 | AT5G54160 | OMT1 | ATOMT1, O-methyltransferase 1, AtCOMT, COMT1, caffeate | 5.5 |
| | | | O-methyltransferase 1, OMT3, O-methyltransferase 3 | |
| 79 | AT5G09830 | BolA2 | BolA-like family protein, homolog of E. coli BolA 2 | 5.2 |
| 80 | AT1G63970 | ISPF | MECPS, 2C-METHYL-D-ERYTHRITOL 2,4-CYCLODIPHOSPHATE | 5.1 |
| | | | SYNTHASE | |
| 81 | AT5G48930 | HCT | hydroxycinnamoyl-CoA shikimate/quinate hydroxycinnamoyl | 5.1 |
| | | | transferase | |
| 82 | AT1G20696 | HMGB3 | NFD03, NFD3 | 5.0 |
| 83 | AT3G57680 | | Peptidase S41 family protein | 5.0 |
| 84 | AT1G04850 | | ubiquitin-associated (UBA)/TS-N domain-containing protein | 5.0 |
| 85 | AT1G67740 | PSBY | YCF32 | 4.9 |
| 86 | AT1G13440 | GAPC2 | GAPC-2, GLYCERALDEHYDE-3-PHOSPHATE DEHYDROGENASE C-2 | 4.9 |
| 87 | AT5G24310 | ABIL3 | ABL interactor-like protein 3 | 4.9 |
| 88 | AT4G38470 | STY46 | ACT-like protein tyrosine kinase family protein | 4.9 |
| 89 | AT3G20550 | DDL | SMAD/FHA domain-containing protein | 4.9 |
| 90 | AT2G33770 | PHO2 | ATUBC24, UBIQUITIN-CONJUGATING ENZYME 24, UBC24, UBIQUITIN- | 4.8 |
| | | | CONJUGATING ENZYME 24 | |
| 91 | AT4G11110 | SPA2 | SPA1-related 2 | 4.8 |
| 92 | AT1G73700 | | MATE efflux family protein | 4.7 |
| 93 | AT3G22400 | LOX5 | PLAT/LH2 domain-containing lipoxygenase family protein | 4.6 |
| 94 | AT1G80820 | CCR2 | ATCCR2 | 4.6 |
| 95 | AT3G30775 | ERD5 | AT-POX, ATPDH, ATPOX, ARABIDOPSIS THALIANA PROLINE | 4.5 |
| | | | OXIDASE, PDH1, proline dehydrogenase 1, PRO1, PRODH, PROLINE | |
| | | | DEHYDROGENASE | |
| 96 | AT4G00440 | TRM15 | GPI-anchored adhesin-like protein, putative (DUF3741) | 4.5 |
| 97 | ATIG10600 | AMSH2 | associated molecule with the SH3 domain of STAM 2 | 4.5 |
| 98 | AT3G08620 | | RNA-binding KH domain-containing protein | 4.4 |
| 99 | AT5G09920 | NRPB4 | RNA polymerase II, Rpb4, core protein | 4.4 |
| 100 | AT1G76940 | | RNA-binding (RRM/RBD/RNP motifs) family protein | 4.4 |
| 101 | AT3G20300 | | extracellular ligand-gated ion channel protein (DUF3537) | 4.4 |
| 102 | AT1G22410 | | Class-II DAHP synthetase family protein | 4.3 |
| 103 | AT3G23530 | | Cyclopropane-fatty-acyl-phospholipid synthase | 4.3 |
| 104 | AT3G54460 | | SNF2 domain-containing protein/helicase domain-containing | 4.3 |
| | | | protein/F-box family protein | |
| 105 | AT4G21110 | | G10 family protein | 4.3 |
| 106 | AT3G24315 | AtSec20 | Sec20 family protein | 4.2 |
| 107 | AT2G22190 | TPPE | Haloacid dehalogenase-like hydrolase (HAD) superfamily protein | 4.2 |
| 108 | AT2G05070 | LHCB2.2 | LHCB2, LIGHT-HARVESTING CHLOROPHYLL B-BINDING 2 | 4.2 |
| 109 | AT3G03910 | GDH3 | glutamate dehydrogenase 3 | 4.1 |
| 110 | AT5G51940 | NRPB6A | RNA polymerase Rpb6 | 4.1 |
| 111 | AT3G59140 | ABCC10 | ATMRP14, multidrug resistance-associated protein 14, | 4.1 |
| | | | MRP14, multidrug resistance-associated protein 14 | |
| 112 | AT3G16360 | AHP4 | HPT phosphotransmitter 4 | 4.1 |
| 113 | AT4G11920 | CCS52A2 | FZRI, FIZZY-RELATED 1 | 4.0 |
| 114 | AT5G15260 | | ribosomal protein L34e superfamily protein | 3.9 |
| 115 | AT5G10350 | | RNA-binding (RRM/RBD/RNP motifs) family protein | 3.9 |
| 116 | AT5G67320 | HOS15 | WD-40 repeat family protein | 3.9 |
| 117 | AT3G08940 | LHCB4.2 | light harvesting complex photosystem II | 3.8 |
| 118 | AT3G15095 | HCF243 | Serine/Threonine-kinase pakA-like protein | 3.8 |
| 119 | AT5G17920 | ATMS1 | ATCIMS, COBALAMIN-INDEPENDENT METHIONINE SYNTHASE, ATMETS | 3.8 |
| 120 | AT4G12800 | PSAL | photosystem I subunit 1 | 3.7 |
| 121 | AT3G13360 | WIP3 | WPP domain interacting protein 3 | 3.7 |
| 122 | AT1G47330 | | methyltransferase, putative (DUF21) | 3.6 |
| 123 | AT4G11910 | NYE2, | STAY-GREEN-like protein | 3.6 |
| | | NONYELLOWING 2, | | |
| | | SGR2, STAY-GREEN 2 | | |
| 124 | AT2G26810 | | Putative methyltransferase family protein | 3.6 |
| 125 | AT1G52570 | PLDALPHA2 | phospholipase D alpha 2 | 3.6 |
| 126 | AT1G71960 | ABCG25 | ATABCG25, Arabidopsis thaliana ATP-binding cassette G25 | 3.6 |
| 127 | AT1G36380 | | transmembrane protein | 3.5 |
| 128 | AT4G32750 | | transmembrane protein | 3.5 |
| 129 | AT2G36750 | UGT73C1 | UDP-glucosyl transferase 73C1 | 3.4 |
| 130 | AT2G40890 | CYP98A3 | REF8, REDUCED EPIDERMAL FLUORESCENCE 8 | 3.4 |
| 131 | AT2G18196 | | Heavy metal transport/detoxification superfamily protein | 3.4 |
| 132 | AT2G28060 | KIN&#946;3 | 5′-AMP-activated protein kinase beta-2 subunit protein | 3.3 |
| 133 | AT2G19860 | HXK2 | ATHXK2, ARABIDOPSIS THALIANA HEXOKINASE 2 | 3.3 |
| 134 | AT5G51040 | SDHAF2 | succinate dehydrogenase assembly factor | 3.2 |
| 135 | AT5G53400 | BOB1 | HSP20-like chaperones superfamily protein | 3.2 |
| 136 | AT1G49350 | | pfkB-like carbohydrate kinase family protein | 3.2 |
| 137 | AT2G30950 | VAR2 | FTSH2 | 3.2 |
| 138 | AT3G15970 | NUP50 protein | Nucleoporin 50 kDa | 3.1 |
| 139 | AT1G15820 | LHCB6 | CP24 | 3.1 |
| 140 | AT2G45695 | URM11 | Ubiquitin related modifier 1 | 3.1 |
| 141 | AT1G79110 | BRG2 | zinc ion binding protein | 3.1 |
| 142 | AT2G36380 | ABCG34 | ATPDR6, PLEIOTROPIC DRUG RESISTANCE 6, PDR6, pleiotropic drug | 3.0 |
| | | | resistance 6 | |
| 143 | AT3G05170 | | Phosphoglycerate mutase family protein | 2.9 |
| 144 | AT5G16570 | GLN1; 4 | glutamine synthetase 1; 4 | 2.9 |
| 145 | AT1G79630 | | Protein phosphatase 2C family protein | 2.9 |
| 146 | AT3G46450 | SEC14 cytosolic factor | | 2.9 |
| | | family protein/ | | |
| | | phosphoglyceride | | |
| | | transfer family protein | | |
| 147 | AT5G48870 | SAD1 | AtLSM5, AtSAD1, LSM5, SM-like 5 | 2.9 |
| 148 | AT2G25910 | | 3′-5′ exonuclease domain-containing protein/K homology domain- | 2.9 |
| | | | containing protein/KH domain-containing protein | |
| 149 | AT5G11760 | | stress response protein | 2.8 |
| 150 | AT4G23840 | | Leucine-rich repeat (LRR) family protein | 2.8 |
| 151 | AT5G17010 | | Major facilitator superfamily protein | 2.8 |
| 152 | AT3G09560 | PAH1 | ATPAH1, PHOSPHATIDIC ACID PHOSPHOHYDROLASE 1 | 2.8 |
| 153 | AT1G79900 | BAC2 | Mitochondrial substrate carrier family protein | 2.8 |
| 154 | AT1G67250 | | Proteasome maturation factor UMP1 | 2.7 |
| 155 | AT3G50360 | CEN2 | ATCEN2, centrin2, CEN1, CENTRIN 1 | 2.7 |
| 156 | AT5G53760 | MLO11 | ATMLO11, MILDEW RESISTANCE LOCUS O 11 | 2.6 |
| 157 | AT5G40640 | | transmembrane protein | 2.6 |
| 158 | AT1G06680 | PSBP-1 | OE23, OXYGEN EVOLVING COMPLEX SUBUNIT 23 KDA, OEE2, OXYGEN- | 2.6 |
| | | | EVOLVING ENHANCER PROTEIN 2, PSII-P, PHOTOSYSTEM II SUBUNIT P | |
| 159 | AT1G03600 | PSB27 | photosystem II family protein | 2.6 |
| 160 | AT1G29450 | SAUR64 | SMALL AUXIN UPREGULATED RNA64 | 2.6 |
| 161 | AT3G20760 | Nse4 | component of Smc5/6 DNA repair complex | 2.6 |
| 162 | AT1G80940 | | Snfl kinase interactor-like protein | 2.6 |
| 163 | AT2G47980 | SCC3 | ATSCC3, SISTER-CHROMATID COHESION PROTEIN 3 | 2.6 |
| 164 | AT2G43840 | UGT74F1 | UDP-glycosyltransferase 74 F1 | 2.5 |
| 165 | AT5G08170 | EMB1873 | ATAIH, AGMATINE IMINOHYDROLASE | 2.5 |
| 166 | AT1G57750 | CYP96A15 | MAH1, MID-CHAIN ALKANE HYDROXYLASE 1 | 2.5 |
| 167 | AT1G77590 | LACS9 | long chain acyl-CoA synthetase 9 | 2.5 |
| 168 | AT1G55610 | BRL1 | BRI1 like | 2.5 |
| 169 | AT1G51740 | SYP81 | ATSYP81, ATUFE1, ARABIDOPSIS THALIANA ORTHOLOG OF YEAST | 2.4 |
| | | | UFEI (UNKNOWN FUNCTION-ESSENTIAL 1), UFE1, ORTHOLOG OF | |
| | | | YEAST UFE1 (UNKNOWN FUNCTION-ESSENTIAL 1) | |
| 170 | AT5G65010 | ASN2 | asparagine synthetase 2 | 2.4 |
| 171 | AT2G25220 | | Protein kinase superfamily protein | 2.4 |
| 172 | AT5G62190 | PRH75 | DEAD box RNA helicase (PRH75) | 2.4 |
| 173 | AT4G23180 | CRK10 | RLK4 | 2.4 |
| 174 | AT4G01050 | TROL | thylakoid rhodanese-like protein | 2.3 |
| 175 | AT5G57960 | Hflx | GTP-binding protein, HflX | 2.3 |
| 176 | AT5G61040 | | hypothetical protein | 2.3 |
| 177 | AT2G32320 | | tRNAHis guanylyltransferase | 2.3 |
| 178 | AT3G06270 | | Protein phosphatase 2C family protein | 2.2 |
| 179 | AT4G21215 | | transmembrane protein | 2.2 |
| 180 | AT3G14840 | LIK1, LysM RLK1- | Leucine-rich repeat transmembrane protein kinase | 2.2 |
| | | interacting kinase 1 | | |
| 181 | AT5G46840 | | RNA-binding (RRM/RBD/RNP motifs) family protein | 2.2 |
| 182 | AT5G50580 | SAE1B | AT-SAE1-2 | 2.2 |
| 183 | AT4G17670 | | senescence-associated family protein (DUF581) | 2.2 |
| 184 | AT2G07050 | CAS1 | cycloartenol synthase 1 | 2.2 |
| 185 | AT4G14960 | TUA6 | Tubulin/FtsZ family protein | 2.2 |
| 186 | AT1G01420 | UGT72B3 | UDP-glucosyl transferase 72B3 | 2.2 |
| 187 | AT1G10140 | | Uncharacterized conserved protein UCP031279 | 2.2 |
| 188 | AT2G18040 | PIN1AT | peptidylprolyl cis/trans isomerase, NIMA-interacting 1 | 2.1 |
| 189 | AT3G62270 | BOR2 | HCO3-transporter family, REQUIRES HIGH BORON 2 | 2.1 |
| 190 | AT5G44070 | CAD1 | ARA8, ATPCS1, ARABIDOPSISTHALIANA PHYTOCHELATIN SYNTHASE | 2.1 |
| | | | 1, PCS1, PHYTOCHELATIN SYNTHASE 1 | |
| 191 | AT1G29500 | SAUR66 | SAUR-like auxin-responsive protein family, , SMALL AUXIN | 2.1 |
| | | | UPREGULATED RNA66 | |
| 192 | AT1G11170 | | lysine ketoglutarate reductase trans-splicing-like protein (DUF707) | 2.1 |
| 193 | AT1G02850 | BGLU11 | beta glucosidase 11 | 2.1 |
| 194 | AT5G47060 | | hypothetical protein (DUF581) | 2.1 |
| 195 | AT3G63140 | CSP41A | chloroplast stem-loop binding protein of 41 kDa | 2.0 |
| 196 | AT5G50110 | | S-adenosyl-L-methionine-dependent methyltransferases superfamily protein | 2.0 |
| 197 | AT5G57700 | | BNR/Asp-box repeat family protein | 2.0 |
| 198 | AT5G57230 | | | 2.0 |
| 199 | AT2G45210 | SAUR36 | SAG201, senescence-associated gene 201 | 2.0 |
| 200 | AT5G01530 | LHCB4.1 | light harvesting complex photosystem II | 2.0 |
| 201 | AT4G39720 | | VQ motif-containing protein | 2.0 |
| 202 | AT4G26950 | | senescence regulator (Protein of unknown function, DUF584) | 2.0 |
| 203 | AT2G36840 | ACR10 | ACT-like superfamily protein | 2.0 |
| 204 | AT5G13290 | CRN | SOL2, SUPPRESSOR OF LLP1 2 | 2.0 |
| 205 | AT2G35170 | | Histone H3 K4-specific methyltransferase SET7/9 family protein | 2.0 |
| 206 | AT5G02380 | MT2B | metallothionein 2B | 1.9 |
| 207 | AT5G63135 | | transcription termination factor | 1.9 |
| 208 | AT3G03780 | MS2 | ATMS2, methionine synthase 2 | 1.9 |
| 209 | AT3G48750 | CDC2 | CDC2A, CDC2AAT, CDK2, CDKA1, CDKA; 1 | 1.9 |
| 210 | AT1G11220 | | cotton fiber, putative (DUF761) | 1.9 |
| 211 | AT1G08380 | PSAO | photosystem I subunit O | 1.9 |
| 212 | AT2G40980 | | Protein kinase superfamily protein | 1.9 |
| 213 | AT4G16146 | | cAMP-regulated phosphoprotein 19-related protein | 1.9 |
| 214 | AT1G16860 | | Ubiquitin-specific protease family C19-related protein | 1.8 |
| 215 | AT1G56190 | | Phosphoglycerate kinase family protein | 1.8 |
| 216 | AT2G44280 | | Major facilitator superfamily protein | 1.8 |
| 217 | AT2G18740 | | Small nuclear ribonucleoprotein family protein | 1.8 |
| 218 | AT2G39050 | EULS3 | ArathEULS3 | 1.8 |
| 219 | AT2G46660 | CYP78A6 | EOD3, enhancer of da1-1 | 1.8 |
| 220 | AT1G76520 | PILS3, PIN-LIKES 3 | Auxin efflux carrier family protein | 1.8 |
| 221 | AT1G76270 | | O-fucosyltransferase family protein | 1.8 |
| 222 | AT2G26650 | KT1 | AKT1, K+ transporter 1, ATAKT1 | 1.8 |
| 223 | AT5G43810 | AGO10 | PNH, PINHEAD, ZLL, ZWILLE | 1.8 |
| 224 | AT2G41380 | | S-adenosyl-L-methionine-dependent methyltransferases superfamily | 1.7 |
| | | | protein | |
| 225 | AT2G46200 | | U11/U12 small nuclear ribonucleoprotein | 1.7 |
| 226 | AT3G50820 | PSBO2 | OEC33, OXYGEN EVOLVING COMPLEX SUBUNIT 33 KDA, PSBO-2, | 1.7 |
| | | | PHOTOSYSTEM II SUBUNIT O-2 | |
| 227 | AT4G05180 | PSBQ-2 | PSBQ, PHOTOSYSTEM II SUBUNIT Q, PSII-Q | 1.7 |
| 228 | AT4G32940 | GAMMA-VPE | GAMMAVPE | 1.7 |
| 229 | AT1G02640 | BXL2 | ATBXL2, BETA-XYLOSIDASE 2 | 1.6 |
| 230 | AT5G52450 | | MATE efflux family protein | 1.6 |
| 231 | AT3G25860 | LTA2 | 2-oxoacid dehydrogenases acyltransferase family protein | 1.6 |
| 232 | AT2G32415 | | Polynucleotidyl transferase, ribonuclease H fold protein with HRDC | 1.6 |
| | | | domain-containing protein | |
| 233 | AT1G73350 | | ankyrin repeat protein | 1.6 |
| 234 | AT1G03475 | LIN2 | ATCPO-I, HEMF1 | 1.6 |
| 235 | AT4G27820 | BGLU9 | beta glucosidase 9 | 1.6 |
| 236 | AT1G12840 | DET3 | ATVH A-C, ARABIDOPSIS THALIANA VACUOLAR ATP SYNTHASE | 1.6 |
| | | | SUBUNIT C | |
| 237 | AT4G01670 | | hypothetical protein | 1.6 |
| 238 | AT4G36940 | NAPRT1 | nicotinate phosphoribosyltransferase 1 | 1.6 |
| 239 | AT5G13980 | | Glycosyl hydrolase family 38 protein | 1.6 |
| 240 | AT4G18360 | GOX3 | Aldolase-type TIM barrel family protein | 1.5 |
| 241 | AT4G21280 | PSBQA | PSBQ-1, PHOTOSYSTEM II SUBUNIT Q-1, PSBQ, PHOTOSYSTEM II | 1.5 |
| | | | SUBUNIT Q | |
| 242 | AT4G12290 | | Copper amine oxidase family protein | 1.5 |
| 243 | AT4G38400 | EXLA2 | ATEXLA2, expansin-like A2, ATEXPL2, ATHEXP BETA | 1.5 |
| | | | 2.2, EXPL2, EXPANSIN L2 | |
| 244 | AT5G57330 | | Galactose mutarotase-like superfamily protein | 1.5 |
| 245 | AT2G43780 | | cytochrome oxidase assembly protein | 1.5 |
| 246 | AT4G09340 | | SPIa/RYanodine receptor (SPRY) domain-containing protein | 1.5 |
| 247 | AT4G36720 | HVA22K | HVA22-like protein K | 1.5 |
| 248 | AT4G21660 | | proline-rich spliceosome-associated (PSP) family protein | 1.5 |
| 249 | AT5G12250 | TUB6 | beta-6 tubulin | 1.5 |
| 250 | AT1G01790 | KEA1 | ATKEA1, K+ EFFLUX ANTI PORTER 1 | 1.5 |
| 251 | AT3G06350 | MEE32 | EMB3004, EMBRYO DEFECTIVE 3004 | 1.4 |
| 252 | AT5G61820 | | stress up-regulated Nod 19 protein | 1.4 |
| 253 | AT2G43750 | OASB | ACS1, ARABIDOPSIS CYSTEINE SYNTHASE 1, ATCS-B, ARABIDOPSIS | 1.4 |
| | | | THALIANA CYSTEIN SYNTHASE-B, CPACS1, CHLOROPLAST | |
| | | | O-ACETYLSERINE SULFHYDRYLASE 1 | |
| 254 | AT1G07470 | | Transcription factor IIA, alpha/beta subunit | 1.4 |
| 255 | AT2G37770 | ChlAKR | AKR4C9, Aldo-keto reductase family 4 member C9 | 1.4 |
| 256 | AT1G29510 | SAUR68 | SAUR67, SMALL AUXIN UPREGULATED RNA 67 | 1.4 |
| 257 | AT4G14600 | | Target SNARE coiled-coil domain protein | 1.4 |
| 258 | AT1G31330 | PSAF | photosystem I subunit F | 1.4 |
| 259 | AT5G43260 | | chaperone protein dnaJ-like protein | 1.4 |
| 260 | AT2G38360 | PRA1.B4 | prenylated RAB acceptor 1.B4 | 1.4 |
| 261 | AT3G05120 | GID1A | ATGID1A, GA INSENSITIVE DWARF1A | 1.4 |
| 262 | AT4G28830 | | S-adenosyl-L-methionine-dependent methyltransferases superfamily | 1.4 |
| | | | protein | |
| 263 | AT3G49645 | | FAD-binding protein | 1.4 |
| 264 | AT1G76080 | CDSP32 | ATCDSP32, ARABIDOPSIS THALIANA CHLOROPLASTIC DROUGHT- | 1.3 |
| | | | INDUCED STRESS PROTEIN OF 32 KD | |
| 265 | AT2G01490 | PAHX | phytanoyl-CoA dioxygenase (PhyH) family protein | 1.3 |
| 266 | AT2G07680 | ABCC13 | ATMRP11, multidrug resistance-associated protein 11, AtABCC13, | 1.3 |
| | | | MRP11, multidrug resistance-associated protein 11 | |
| 267 | AT4G23150 | CRK7 | cysteine-rich RLK (RECEPTOR-like protein kinase) 7 | 1.3 |
| 268 | AT2G47060 | PTI1-4 | Protein kinase superfamily protein | 1.3 |
| 269 | AT4G01370 | MPK4 | ATMPK4, MAP kinase 4, MAPK4 | 1.3 |
| 270 | AT1G5445O | | Calcium-binding EF-hand family protein | 1.3 |
| 271 | AT2G41830 | | Uncharacterized protein | 1.3 |
| 272 | AT2G18600 | | Ubiquitin-conjugating enzyme family protein | 1.3 |
| 273 | AT4G24400 | CIPK8 | ATCIPK8, PKS11, PROTEIN KINASE 11, SnRK3.13, SNF1-RELATED | 1.3 |
| | | | PROTEIN KINASE 3.13 | |
| 274 | AT4G28706 | | pfkB-like carbohydrate kinase family protein | 1.3 |
| 275 | AT1G21210 | WAK4 | wall associated kinase 4 | 1.3 |
| 276 | AT5G53800 | | nucleic acid-binding protein | 1.3 |
| 277 | AT5G10840 | EMP1 | Endomembrane protein 70 protein family | 1.3 |
| 278 | AT1G33840 | | LURP-one-like protein (DUF567) | 1.3 |
| 279 | AT5G63030 | GRXC1 | Thioredoxin superfamily protein | 1.3 |
| 280 | AT2G44130 | | KFB39, Kelch-domain-containing F-box protein 39, KMD3, KISS ME | 1.2 |
| | | | DEADLY 3 | |
| 281 | AT5G04830 | | Nuclear transport factor 2 (NTF2) family protein | 1.2 |
| 282 | AT3G15360 | TRX-M4 | ATHM4, ATM4, ARABIDOPSIS THIOREDOXIN M-TYPE 4 | 1.2 |
| 283 | AT4G14880 | OASA1 | ATCYS-3A, CYTACS1, OLD3, ONSET OF LEAF DEATH 3 | 1.2 |
| 284 | AT2G21390 | | Coatomer, alpha subunit | 1.2 |
| 285 | AT5G10780 | | ER membrane protein complex subunit-like protein | 1.2 |
| 286 | AT5G46910 | | Transcription factor jumonji (jmj) family protein/zinc finger (C5HC2 type) | 1.2 |
| | | | family protein | |
| 287 | AT1G65820 | | microsomal glutathione s-transferase | 1.2 |
| 288 | AT1G65840 | PAO4 | ATPAO4, polyamine oxidase 4 | 1.2 |
| 289 | AT1G64640 | ENODL8 | AtENODL8 | 1.2 |
| 290 | AT5G49830 | EXO84B | exocyst complex component 84B | 1.2 |
| 291 | AT4G13345 | MEE55 | Serinc-domain containing serine and sphingolipid biosynthesis | 1.1 |
| | | | protein | |
| 292 | AT5G63890 | HDH | ATHDH, histidinol dehydrogenase, HISN8, HISTIDINE BIOSYNTHESIS 8 | 1.1 |
| 293 | AT3G52960 | | Thioredoxin superfamily protein | 1.1 |
| 294 | AT1G59950 | | NAD(P)-linked oxidoreductase superfamily protein | 1.1 |
| 295 | AT3G57050 | CBL | cystathionine beta-lyase | 1.1 |
| 296 | AT5G52230 | MBD13 | methyl-CPG-binding domain protein 13 | 1.1 |
| 297 | AT2G27510 | FD3 | ATFD3, ferredoxin 3 | 1.1 |
| 298 | AT5G15780 | | Pollen Ole e l allergen and extensin family protein | 1.1 |
| 299 | AT1G67570 | | zinc finger CONSTANS-like protein (DUF3537) | 1.1 |
| 300 | AT5G06230 | TBL9 | TRICHOME BIREFRINGENCE-LIKE 9 | 1.1 |
| 301 | AT4G27270 | | Quinone reductase family protein | 1.1 |
| 302 | AT1G15410 | | aspartate-glutamate racemase family | 1.1 |
| 303 | AT3G47000 | | Glycosyl hydrolase family protein | 1.1 |
| 304 | AT5G22740 | CSLA02 | ATCSLA02, ARABIDOPSIS THALIANA CELLULOSE SYNTHASE-LIKE | 1.1 |
| | | | A02, ATCSLA2, ARABIDOPSIS THALIANA CELLULOSE SYNTHASE-LIKE | |
| | | | A2, CSLA2, CELLULOSE SYNTHASE-LIKE A 2 | |
| 305 | AT4G39400 | BRI1 | ATBRI1, BIN1, BR INSENSITIVE 1, CBB2, CABBAGE 2, DWF2, DWARF 2 | 1.1 |
| 306 | AT1G77460 | CSI3 | CELLULOSE SYNTHASE INTERACTIVE 3 | 1.0 |
| 307 | AT5G14880 | KUP8 | Potassium transporter family protein | 1.0 |
| 308 | AT3G47470 | LHCA4 | CAB4 | 1.0 |
| 309 | AT4G39640 | GGT1 | gamma-glutamyl transpeptidase 1 | 1.0 |
| 310 | AT2G06010 | ORG4 | OBP3-responsive protein 4 (ORG4) | 1.0 |
| 311 | AT5G61220 | | LYR family of Fe/S cluster biogenesis protein | 1.0 |
| 312 | AT3G28740 | CYP81D11 | Cytochrome P450 superfamily protein | 1.0 |
| 313 | AT4G33950 | OST1 | ATOST1, OPEN STOMATA l, P44, SNRK2-6, SUCROSE NONFERMENTING | 1.0 |
| | | | 1-RELATED PROTEIN KINASE 2-6, SNRK2.6, SNFl-RELATED PROTEIN | |
| | | | KINASE 2.6, SRK2E | |
| 314 | AT1G01560 | MPK11 | ATMPK11, MAP kinase 11 | 1.0 |
| 315 | AT3G23090 | WDL3 | TPX2 (targeting protein for Xklp2) protein family | 1.0 |
| 316 | AT4G09750 | | NAD(P)-binding Rossmann-fold superfamily protein | 1.0 |
| 317 | AT3G54890 | LHCA1 | chlorophyll a-b binding protein 6 | 1.0 |
| 318 | AT3G46780 | PTAC16 | plastid transcriptionally active 16 | 0.9 |
| 319 | AT5G40440 | MKK3 | ATMKK3, mitogen-activated protein kinase kinase 3 | 0.9 |
| 320 | AT4G23230 | CRK15 | cysteine-rich RECEPTOR-like kinase | 0.9 |
| 321 | AT4G10840 | KLCR1 | Tetratricopeptide repeat (TPR)-like superfamily protein | 0.9 |
| 322 | AT4G37400 | CYP81F3 | cytochrome P450, family 81, subfamily F, polypeptide 3 | 0.9 |
| 323 | AT5G67570 | DG1 | EMB1408, embryo defective 1408, EMB246, EMBRYO DEFECTIVE 246 | 0.9 |
| 324 | AT2G41050 | | PQ-loop repeat family protein/transmembrane family protein | 0.9 |
| 325 | AT1G01620 | PIP1C | PIP1; 3, PLASMA MEMBRANE INTRINSIC PROTEIN 1; 3, TMP-B | 0.9 |
| 326 | AT3G21055 | PSBTN | photosystem II subunit T | 0.9 |
| 327 | AT4G15910 | DI21 | ATDI21, drought-induced 21 | 0.9 |
| 328 | AT1G53470 | MSL4 | mechanosensitive channel of small conductance-like 4 | 0.9 |
| 329 | AT1G14290 | SBH2 | AtSBH2 | 0.9 |
| 330 | AT2G42960 | | Protein kinase superfamily protein | 0.9 |
| 331 | AT1G71080 | | RNA polymerase II transcription elongation factor | 0.9 |
| 332 | AT5G63970 | RGLG3 | Copine (Calcium-dependent phospholipid-binding protein) family | 0.9 |
| 333 | AT5G35100 | | Cyclophilin-like peptidyl-prolyl cis-trans isomerase family protein | 0.9 |
| 334 | AT1G14000 | VIK | VHl-interacting kinase | 0.9 |
| 335 | AT3G47050 | | Glycosyl hydrolase family protein | 0.8 |
| 336 | AT1G17600 | | Disease resistance protein (TIR-NBS-LRR class) family | 0.8 |
| 337 | AT3G12290 | | Amino acid dehydrogenase family protein | 0.8 |
| 338 | AT5G65620 | OOP | Zincin-like metalloproteases family protein, organellar | 0.8 |
| | | | oligopeptidase, TOP1, thimet metalloendopeptidase 1 | |
| 339 | AT4G28750 | PSAE-1 | Photosystem 1 reaction centre subunit IV/PsaE protein | 0.8 |
| 340 | AT1G65800 | RK2 | ARK2, receptor kinase 2, AtARK2 | 0.8 |
| 341 | AT1G75690 | LQY1 | DnaJ/Hsp40 cysteine-rich domain superfamily protein | 0.8 |
| 342 | AT4G01150 | CURT1A | CURVATURE THYLAKOID 1A-like protein | 0.8 |
| 343 | AT1G03680 | THM1 | ATHM1, thioredoxin M-type 1, ATM1, ARABIDOPSIS THIOREDOXIN | 0.8 |
| | | | M-TYPE 1, TRX-M1, THIOREDOXIN M-TYPE 1 | |
| 344 | AT4G33040 | | Thioredoxin superfamily protein | 0.8 |
| 345 | AT4G32260 | PDE334 | ATPase, F0 complex, subunit B/B′, bacterial/chloroplast | 0.8 |
| 346 | AT2G34730 | | myosin heavy chain-like protein | 0.8 |
| 347 | AT3G09830 | PCRK1 | Protein kinase superfamily protein | 0.8 |
| 348 | AT4G09510 | CINV2 | A/N-lnvl, alkaline/neutral invertase 1 | 0.8 |
| 349 | AT1G04820 | TUA4 | TOR2, TORTIFOLIA 2 | 0.8 |
| 350 | AT5G62200 | | Embryo-specific protein 3, (ATS3) | 0.8 |
| 351 | AT1G17170 | GSTU24 | ATGSTU24, glutathione S-transferase TAU 24, GST, Arabidopsis thaliana | 0.8 |
| | | | Glutathione S-transferase (class tau) 24 | |
| 352 | AT5G4647O | RPS6 | disease resistance protein (TIR-NBS-LRR class) family | 0.8 |
| 353 | AT5G6514O | TPPJ | Haloacid dehalogenase-like hydrolase (HAD) superfamily protein | 0.8 |
| 354 | AT5G64040 | PSAN | photosystem 1 reaction center subunit PSI-N, chloroplast, putative/ | 0.8 |
| | | | PSI-N, putative (PSAN) | |
| 355 | AT1G65930 | cICDH | cytosolic NADP+-dependent isocitrate dehydrogenase | 0.8 |
| 356 | AT5G2381O | AAP7 | amino acid permease 7 | 0.7 |
| 357 | AT2G24395 | | chaperone protein dnaJ-like protein | 0.7 |
| 358 | AT2G42220 | | Rhodanese/Cell cycle control phosphatase superfamily protein | 0.7 |
| 359 | AT1G14910 | | ENTH/ANTH/VHS superfamily protein | 0.7 |
| 360 | AT1G72230 | | Cupredoxin superfamily protein | 0.7 |
| 361 | AT4G11530 | CRK34 | cysteine-rich RLK (RECEPTOR-like protein kinase) 34 | 0.7 |
| 362 | AT3G14690 | CYP72A15 | cytochrome P450, family 72, subfamily A, polypeptide 15 | 0.7 |
| 363 | AT4G09570 | CPK4 | ATCPK4 | 0.7 |
| 364 | AT5G28080 | WNK9 | Protein kinase superfamily protein | 0.7 |
| 365 | AT1G01180 | | S-adenosyl-L-methionine-dependent methyltransferases superfamily protein | 0.7 |
| 366 | AT1G67500 | REV3 | ATREV3, recovery protein 3 | 0.7 |
| 367 | AT3G47090 | | Leucine-rich repeat protein kinase family protein | 0.7 |
| 368 | AT2G36800 | DOGT1 | UGT73C5, UDP-GLUCOSYL TRANSFERASE 73C5 | 0.7 |
| 369 | AT5G43940 | HOT5 | ADH2, ALCOHOL DEHYDROGENASE 2, ATGSNOR1, GSNOR, S- | 0.7 |
| | | | NITROSOGLUTATHIONE REDUCTASE, PAR2, PARAQUAT RESISTANT 2 | |
| 370 | AT1G22610 | | C2 calcium/lipid-binding plant phosphoribosyltransferase family protein | 0.7 |
| 371 | AT2G41740 | VLN2 | ATVLN2 | 0.7 |
| 372 | AT5G50100 | | Putative thiol-disulfide oxidoreductase DCC | 0.7 |
| 373 | AT4G03110 | RBP-DR1 | AtBRN1, AtRBP-DR1, RNA-binding protein-defense related 1, | 0.7 |
| | | | BRN1, Bruno-like 1 | |
| 374 | AT5G47770 | FPS1 | farnesyl diphosphate synthase 1 | 0.7 |
| 375 | AT1G78460 | | SOUL heme-binding family protein | 0.7 |
| 376 | AT5G41000 | YSL4 | AtYSL4 | 0.7 |
| 377 | AT1G33490 | | E3 ubiquitin-protein ligase | 0.7 |
| 378 | AT2G06520 | PSBX | photosystem II subunit X | 0.7 |
| 379 | AT1G76140 | | Prolyl oligopeptidase family protein | 0.7 |
| 380 | AT1G55670 | PSAG | photosystem I subunit G | 0.6 |
| 381 | AT5G01880 | DAFL2, | RING/U-box superfamily protein | 0.6 |
| | | DAF-Like gene 2 | | |
| 382 | AT4G22920 | NYE1 | ATNYE1, NON-YELLOWING 1, SGR1, STAY-GREEN 1, SGR, STAY-GREEN | 0.6 |
| 383 | AT2G26910 | ABCG32 | ATPDR4, PLEIOTROPIC DRUG RESISTANCE 4, AtABCG32, PDR4, | 0.6 |
| | | | pleiotropic drug resistance 4, PEC1, PERMEABLE CUTICLE 1 | |
| 384 | AT1G80380 | | P-loop containing nucleoside triphosphate hydrolases superfamily protein | 0.6 |
| 385 | AT3G06890 | | transmembrane protein | 0.6 |
| 386 | AT3G46460 | UBC13 | ubiquitin-conjugating enzyme 13 | 0.6 |
| 387 | AT1G13195 | | RING/U-box superfamily protein | 0.6 |
| 388 | AT1G17710 | PEPC1 | AtPEPCl, Arabidopsis thaliana phosphoethanolamine/phosphocholine | 0.6 |
| | | | phosphatase 1 | |
| 389 | AT4G28070 | | AFGl-like ATPase family protein | 0.6 |
| 390 | AT3G13620 | PUT4 | Amino acid permease family protein | 0.6 |
| 391 | AT4G33540 | | metallo-beta-lactamase family protein | 0.6 |
| 392 | AT2G39705 | RTFL8 | DVL11, DEVIL 11 | 0.6 |
| 393 | AT2G43820 | UGT74F2 | ATSAGTl1 Arabidopsis thaliana salicylic acid glucosyltransferase 1, GT, | 0.6 |
| | | | SAGT1, salicylic acid glucosyltransferase 1, SGT1, UDP-glucose: | |
| | | | salicylic acid glucosyltransferase l | |
| 394 | AT1G74410 | | RING/U-box superfamily protein | 0.6 |
| 395 | AT4G24670 | TAR2 | tryptophan aminotransferase related 2 | 0.6 |
| 396 | AT1G59670 | GSTU15 | ATGSTU15, glutathione S-transferase TAU 15 | 0.6 |
| 397 | AT2G22170 | PLAT2 | Lipase/lipooxygenase, PLAT/LH2 family protein | 0.6 |
| 398 | AT1G80860 | PLMT | ATPLMT, ARABIDOPSIS PHOSPHOLIPID N-METHYLTRANSFERASE | 0.6 |
| 399 | AT2G35130 | | Tetratricopeptide repeat (TPR)-like superfamily protein | 0.6 |
| 400 | AT1G16110 | WAKL6 | wall associated kinase-like 6 | 0.6 |
| 401 | AT4G38060 | CCI2, Clavata complex | hypothetical protein | 0.6 |
| | | interactor 2 | | |
| 402 | AT1G7653O | PILS4, PIN-LIKES 4 | Auxin efflux carrier family protein | 0.6 |
| 403 | AT2G26400 | ARD3 | ARD, ACIREDUCTONE DIOXYGENASE, ATARD3, acireductone | 0.6 |
| | | | dioxygenase 3 | |
| 404 | AT1G48600 | PMEAMT | AtPMEAMT | 0.6 |
| 405 | AT1G16260 | | Wall-associated kinase family protein | 0.6 |
| 406 | AT2G34420 | LHB1B2 | LHCB1.5, PHOTOSYSTEM II LIGHT HARVESTING COMPLEX GENE 1.5 | 0.6 |
| 407 | AT4G23400 | PIP1; 5 | PIPID | 0.6 |
| 408 | AT2G30550 | DALL3, DAD1-Like | alpha/beta-Hydrolases superfamily protein | 0.6 |
| | | Lipase 3 | | |
| 409 | AT1G24170 | LGT9 | Nucleotide-diphospho-sugar transferases superfamily protein | 0.6 |
| 410 | AT1G13750 | ATPAP1, | Purple acid phosphatases superfamily protein | 0.5 |
| | | ARABIDOPSIS | | |
| | | THALIANA | | |
| | | PURPLE ACID | | |
| | | PHOSPHATASE 1, | | |
| | | PAP1, PURPLE ACID | | |
| | | PHOSPHATASE 1 | | |
| 411 | AT4G04640 | ATPC1 | ATPase, F1 complex, gamma subunit protein | 0.5 |
| 412 | AT4G24460 | CLT2 | CRT (chloroquine-resistance transporter)-like transporter 2 | 0.5 |
| 413 | AT3G63010 | GID1B | ATGIDIB | 0.5 |
| 414 | AT5G59290 | UXS3 | ATUXS3 | 0.5 |
| 415 | AT5G66850 | MAPKKK5 | mitogen-activated protein kinase kinase kinase 5 | 0.5 |
| 416 | AT4G02770 | PSAD-1 | photosystem I subunit D-1 | 0.5 |
| 417 | AT1G30380 | PSAK | photosystem I subunit K | 0.5 |
| 418 | AT1G28440 | HSL1 | HAESA-like 1 | 0.5 |
| 419 | AT3G21600 | | Senescence/dehydration-associated protein-like protein | 0.5 |
| 420 | AT1G21270 | WAK2 | wall-associated kinase 2 | 0.5 |
| 421 | AT4G34150 | | Calcium-dependent lipid-binding (CaLB domain) family protein | 0.5 |
| 422 | AT3G19270 | CYP707A4 | cytochrome P450, family 707, subfamily A, polypeptide 4 | 0.5 |
| 423 | AT5G12010 | | nuclease | 0.5 |
| 424 | AT2G24170 | | Endomembrane protein 70 protein family | 0.5 |
| 425 | AT1G07650 | | Leucine-rich repeat transmembrane protein kinase | 0.5 |
| 426 | AT5G13120 | Pnsl5 | ATCYP20-2, ARABIDOPSIS THALIANA CYCLOPHILIN 20-2, CYP20-2, | 0.5 |
| | | | cyclophilin 20-2 | |
| 427 | AT5G33320 | CUE1 | ARAPPT, ARABIDOPSIS THALIANA | 0.5 |
| | | | PHOSPHATE/PHOSPHOENOLPYRUVATE | |
| | | | TRANSLOCATOR, PPT, PHOSPHOENOLPYRUVATE/PHOSPHATE | |
| | | | TRANSLOCATOR | |
| 428 | AT4G10340 | LHCB5 | light harvesting complex of photosystem II 5 | 0.5 |
| 429 | AT3G61470 | LHCA2 | photosystem 1 light harvesting complex protein | 0.5 |
| 430 | AT5G43150 | | elongation factor | 0.5 |
| 431 | AT5G44060 | | embryo sac development arrest protein | 0.5 |
| 432 | AT5G16400 | TRXF2 | ATF2 | 0.5 |
| 433 | AT5G14200 | IMD1 | ATIMD1, ARABIDOPSIS ISOPROPYLMALATE DEHYDROGENASE 1 | 0.5 |
| 434 | AT1G05630 | 5PTASE13 | AT5PTASE13, Arabidopsis thaliana inositol-polyphosphate 5- | 0.5 |
| | | | phosphatase 13 | |
| 435 | AT1G50010 | TUA2 | tubulin alpha-2 chain | 0.5 |
| 436 | AT3G17180 | scpl33 | serine carboxypeptidase-like 33 | 0.5 |
| 437 | AT2G36310 | URH1 | NSH1, nucleoside hydrolase 1 | 0.5 |
| 438 | AT1G63840 | | RING/U-box superfamily protein | 0.5 |
| 439 | AT1G20110 | | FREE1, FYVE domain protein required for endosomal sorting 1, FYVE1, | 0.5 |
| | | | FYVE-domain protein 1 | |
| 440 | AT1G79270 | ECT8 | evolutionarily conserved C-terminal region 8 | 0.4 |
| 441 | AT2G30570 | PSBW | photosystem II reaction center W | 0.4 |
| 442 | AT3G50910 | | netrin receptor DCC | 0.4 |
| 443 | AT2G42070 | NUDX23 | ATNUDT23, ARABIDOPSIS THALIANA NUDIX HYDROLASE HOMOLOG | 0.4 |
| | | | 23, ATNUDX23, nudix hydrolase homolog 23 | |
| 444 | AT5G66570 | PSBO1 | MSP-1, MANGANESE-STABILIZING PROTEIN 1, OE33, OXYGEN | 0.4 |
| | | | EVOLVING COMPLEX 33 KILODALTON PROTEIN, OEE1, 33 KDA OXYGEN | |
| | | | EVOLVING POLYPEPTIDE 1, OEE33, OXYGEN EVOLVING ENHANCER | |
| | | | PROTEIN 33, PSBO-1, PS II OXYGEN-EVOLVING COMPLEX 1 | |
| 445 | AT1G67060 | | peptidase M50B-like protein | 0.4 |
| 446 | AT1G34210 | SERK2 | ATSERK2 | 0.4 |
| 447 | AT2G34430 | LHB1B1 | LHCB1.4, LIGHT-HARVESTING CHLOROPHYLL-PROTEIN COMPLEX II | 0.4 |
| | | | SUBUNIT B1 | |
| 448 | AT3G52750 | FTSZ2-2 | Tubulin/FtsZ family protein | 0.4 |
| 449 | AT3G12780 | PGK1 | phosphoglycerate kinase 1 | 0.4 |
| 450 | AT4G34490 | CAP1 | ATCAP1, cyclase associated protein 1, CAP 1 | 0.4 |
| 451 | AT2G38120 | AUX1 | AtAUX1, MAP1, MODIFIER OF ARF7/NPH4 PHENOTYPES 1, PIR1, | 0.4 |
| | | | WAV5, WAVY ROOTS 5 | |
| 452 | AT1G12990 | | beta-1, 4-N-acetylglucosaminyltransferase family protein | 0.4 |
| 453 | AT5G39320 | UDG4 | UDP-glucose 6-dehydrogenase family protein | 0.4 |
| 454 | AT5G06750 | APD8 | Protein phosphatase 2C family protein | 0.4 |
| 455 | AT5G11000 | | hypothetical protein (DUF868) | 0.4 |
| 456 | AT1G61520 | LHCA3 | PSI type III chlorophyll a/b-binding protein | 0.4 |
| 457 | AT1G07000 | EXO70B2 | ATEXO70B2, exocyst subunit exo70 family protein B2 | 0.4 |
| 458 | AT5G07030 | | Eukaryotic aspartyl protease family protein | 0.4 |
| 459 | AT1G59700 | GSTU16 | ATGSTU16, glutathione S-transferase TAU 16 | 0.4 |
| 460 | AT2G20260 | PSAE-2 | photosystem I subunit E-2 | 0.4 |
| 461 | AT2G39740 | HESO1 | Nucleotidyltransferase family protein | 0.4 |
| 462 | AT3G15760 | | cytochrome P450 family protein | 0.4 |
| 463 | AT2G33730 | | P-loop containing nucleoside triphosphate hydrolases superfamily protein | 0.4 |
| 464 | AT2G36330 | CASPL4A3 | CASP-like protein 4A3, Uncharacterized protein family (UPFO497) | 0.4 |
| 465 | AT5G14540 | | basic salivary proline-rich-like protein (DUF1421) | 0.4 |
| 466 | AT4G13510 | AMT1;1 | ATAMT1;1, ATAMT1, ARABIDOPSIS THALIANA AMMONIUM | 0.4 |
| | | | TRANSPORT 1 | |
| 467 | AT1G29910 | CAB3 | AB180, LHCB1.2, LIGHT HARVESTING CHLOROPHYLL A/B BINDING | 0.4 |
| | | | PROTEIN 1.2 | |
| 468 | AT2G31810 | | ACT domain-containing small subunit of acetolactate synthase | 0.4 |
| | | | protein | |
| 469 | AT1G52190 | AtNPF1.2, NPF1.2, | Major facilitator superfamily protein | 0.4 |
| | | NRT1/PTR family 1.2, | | |
| | | NRT1.11, nitrate | | |
| | | transporter 1.11 | | |
| 470 | AT5G01240 | LAX1 | like AUXIN RESISTANT 1 | 0.4 |
| 471 | AT5G64090 | | hyccin | 0.4 |
| 472 | AT4G38540 | | FAD/NAD(P)-binding oxidoreductase family protein | 0.4 |
| 473 | AT3G25510 | | disease resistance protein (TIR-NBS-LRR class) family protein | 0.4 |
| 474 | AT1G75590 | SAUR52 | SAUR-like auxin-responsive protein family, SMALL AUXIN | 0.4 |
| | | | UPREGULATED RNA 52 | |
| 475 | AT5G09930 | ABCF2 | ABC transporter family protein | 0.4 |
| 476 | AT2G14740 | VSR3 | ATVSR3, vaculolar sorting receptor 3, BP80-2;2, binding protein of 80 kDa 2;2, | 0.4 |
| | | | VSR2;2, VACUOLAR SORTING RECEPTOR 2;2 | |
| 477 | AT5G66200 | ARO2 | armadillo repeat only 2 | 0.4 |
| 478 | AT1G31540 | | Disease resistance protein (TIR-NBS-LRR class) family | 0.4 |
| 479 | AT5G62900 | | basic-leucine zipper transcription factor K | 0.3 |
| 480 | AT3G49350 | | Ypt/Rab-GAP domain of gyp1p superfamily protein | 0.3 |
| 481 | AT5G50375 | CPI1 | cyclopropyl isomerase | 0.3 |
| 482 | AT3G05520 | CPA | AtCPA | 0.3 |
| 483 | AT4G36640 | | Sec14p-like phosphatidylinositol transfer family protein | 0.3 |
| 484 | AT3G62110 | | Pectin lyase-like superfamily protein | 0.3 |
| 485 | AT4G36040 | J11 | DJC23, DNA J protein C23 | 0.3 |
| 486 | AT3G56440 | ATG18D | ATATG18D, homolog of yeast autophagy 18 (ATG18) D | 0.3 |
| 487 | AT3G05350 | | Metallopeptidase M24 family protein | 0.3 |
| 488 | AT3G52340 | SPP2 | ATSPP2, SUCROSE-PHOSPHATASE 2 | 0.3 |
| 489 | AT1G34750 | | Protein phosphatase 2C family protein | 0.3 |
| 490 | AT5G47870 | RAD52-2 | ODB2, Organellar DNA-Binding protein 2, RAD52-2B | 0.3 |
| 491 | AT4G22380 | | Ribosomal protein L7Ae/L30e/S12e/Gadd45 family protein | 0.3 |
| 492 | AT5G46110 | APE2 | TPT, triose-phosphate &#8260; phosphate translocator | 0.3 |
| 493 | AT3G63470 | scpl40 | serine carboxypeptidase-like 40 | 0.3 |
| 494 | AT4G39030 | EDS5 | SCORD3, susceptible to coronatine-deficient Pst DC3000 3, SID1, | 0.3 |
| | | | SALICYLIC ACID INDUCTION DEFICIENT 1 | |
| 495 | AT3G60160 | ABCC9 | ATMRP9, multidrug resistance-associated protein 9, MRP9, multidrug | 0.3 |
| | | | resistance-associated protein 9 | |
| 496 | AT5G53550 | YSL3 | ATYSL3, YELLOW STRIPE LIKE 3 | 0.3 |
| 497 | AT4G21190 | emb1417 | Pentatricopeptide repeat (PPR) superfamily protein | 0.3 |
| 498 | AT3G16140 | PSAH-1 | photosystem I subunit H-1 | 0.3 |
| 499 | AT2G36360 | | Galactose oxidase/kelch repeat superfamily protein | 0.3 |
| 500 | AT2G04630 | NRPB6B | RNA polymerase Rpb6 | 0.3 |
| 501 | AT5G58220 | TTL | ALNS, allantoin synthase | 0.3 |
| 502 | AT2G45290 | TKL2 | Transketolase | 0.3 |
| 503 | AT1G13320 | PP2AA3 | protein phosphatase 2A subunit A3 | 0.3 |
| 504 | AT3G58100 | PDCB5 | plasmodesmata callose-binding protein 5 | 0.3 |
| 505 | AT1G20780 | SAUL1 | ATPUB44, ARABIDOPSIS THALIANA PLANT U-BOX 44, PUB44, | 0.3 |
| | | | PLANT U-BOX 44 | |
| 506 | AT4G21380 | RK3 | ARK3, receptor kinase 3 | 0.3 |
| 507 | AT4G20230 | | terpenoid synthase superfamily protein | 0.3 |
| 508 | AT3G17410 | | Protein kinase superfamily protein | 0.3 |
| 509 | AT2G40600 | | appr-1-p processing enzyme family protein | 0.3 |
| 510 | AT1G28580 | | GDSL-like Lipase/Acylhydrolase superfamily protein | 0.3 |
| 511 | AT4G23130 | CRK5 | RLK6, RECEPTOR-LIKE PROTEIN KINASE 6 | 0.3 |
| 512 | AT4G27830 | BGLU10 | AtBGLU10 | 0.3 |
| 513 | AT2G25520 | | Drug/metabolite transporter superfamily protein | 0.3 |
| 514 | AT1G34130 | STT3B | staurosporin and temperature sensitive 3-like b | 0.2 |
| 515 | AT4G29440 | | Regulator of Vps4 activity in the MVB pathway protein | 0.2 |
| 516 | AT1G77490 | TAPX | thylakoidal ascorbate peroxidase | 0.2 |
| 517 | AT4G38660 | | Pathogenesis-related thaumatin superfamily protein | 0.2 |
| 518 | AT3G29360 | UGD2 | UDP-glucose 6-dehydrogenase family protein | 0.2 |
| 519 | AT5G62580 | | ARM repeat superfamily protein | 0.2 |
| 520 | AT1G16670 | | Protein kinase superfamily protein | 0.2 |
| 521 | AT4G09010 | TL29 | APX4, ascorbate peroxidase 4 | 0.2 |
| 522 | AT3G60690 | SAUR59 | SAUR-like auxin-responsive protein family, SMALL AUXIN | 0.2 |
| | | | UPREGULATED RNA 59 | |
| 523 | AT2G37550 | AGD7 | ASP1, yeast pde1 sup, pressor 1 | 0.2 |
| 524 | AT5G11250 | | Disease resistance protein (TIR-NBS-LRR class) | 0.2 |
| 525 | AT5G19780 | TUA5 | tubulin alpha-5 | 0.2 |
| 526 | AT1G55910 | ZIP11 | zinc transporter 11 precursor | 0.2 |
| 527 | AT5G24870 | | RING/U-box superfamily protein | 0.2 |
| 528 | AT3G22840 | ELIP1 | ELIP | 0.2 |
| 529 | AT5G19770 | TUA3 | tubulin alpha-3 | 0.2 |
| 530 | AT1G34630 | | transmembrane protein | 0.2 |
| 531 | AT3G55260 | HEXO1 | ATHEX2 | 0.2 |
| 532 | AT4G02420 | | LecRK-IV.4, L-type lectin receptor kinase IV.4 | 0.2 |
| 533 | AT1G69730 | | Wall-associated kinase family protein | 0.2 |
| 534 | AT1G66880 | | Protein kinase superfamily protein | 0.1 |
| 535 | AT4G23140 | CRK6 | cysteine-rich RLK (RECEPTOR-like protein kinase) 6 | 0.1 |
| 536 | AT2G31020 | ORP1A | OSBP(oxysterol binding protein)-related protein 1A | 0.1 |
| 537 | AT2G16950 | TRN1 | ATTRN1, TRANSPORTIN 1 | 0.1 |
| 538 | AT5G48380 | BIR1 | BAK1-interacting receptor-like kinase 1 | 0.1 |
| 539 | AT5G25100 | | Endomembrane protein 70 protein family | 0.1 |
| 540 | AT1G21250 | WAK1 | AtWAK1, PRO25 | 0.1 |
| 541 | AT5G22770 | alpha-ADR | alpha-adaptin | 0.1 |
| 542 | AT5G60900 | RLK1 | receptor-like protein kinase 1 | 0.1 |
| 543 | AT1G65790 | RK1 | ARK1, receptor kinase 1 | 0.1 |
| 544 | AT5G35200 | | ENTH/ANTH/VHS superfamily protein | 0.1 |
| 545 | AT2G42900 | | Plant basic secretory protein (BSP) family protein | 0.1 |
| 546 | AT3G54100 | | O-fucosyltransferase family protein | 0.0 |
| 547 | AT4G14690 | ELIP2 | Chlorophyll A-B binding family protein | 0.0 |

Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

What is claimed is:

1. A modified plant cell, said modified plant cell comprising a modification that inhibits expression of hb75.

2. The modified plant cell of claim 1, further comprising a modification such that expression of a gene selected from Table 4 is altered.

3. The modified plant cell of claim 2, wherein the gene is nf-ya3, and wherein expression of nf-ya3 is inhibited.

4. A plant comprising plant cells of claim 1.

5. The plant of claim 4, wherein the plant exhibits at least one of: an increase in Nitrogen (N) uptake, increased biomass, an increased harvest index, an increased Total nitrogen utilization (NUtE), or an increased total Grain NUtE, relative to a plant that does not comprise a modification that inhibits expression of hb75.

6. The plant of claim 5, wherein the plant exhibits the increased biomass.

7. The plant of claim 5, wherein the plant is a maize plant.

8. The plant of claim 7, wherein the increased biomass comprises increased grain mass.

9. A method comprising modifying a plant comprising disrupting expression hb75 such that the plant exhibits at least one of an increase in Nitrogen (N) uptake, increased biomass, an increased harvest index, an increased Total nitrogen utilization (NUtE), or an increased total Grain NUtE, relative to a plant that does not comprise a modification that inhibits expression of hb75.

10. The method of claim 9, wherein the plant further comprises a such that expression of a gene selected from Table 4 is altered.

11. The method of claim 10, wherein the gene is nf-ya3, and wherein expression of nf-ya3 is inhibited.

12. The method of claim 9, wherein the plant is a maize plant.

13. The method of claim 10, wherein the plant is a maize plant.

14. The method of claim 11, wherein the plant is a maize plant.
